(19) United States
(12) Patent Application Publication
(10) Pub. No.: US 2006/0277619 A1
(43) Pub. Date: Dec. 7, 2006

(54) Method for identifying herbicidally active substances

(75) Inventor: Gunnar PLESCH, Potsdam (DE)

Correspondence Address: CONNOLLY BOVE LODGE & HUTZ, LLP, P O BOX 2207, WILMINGTON, DE, 19899, US

(73) Assignee: Metanomics GmbH & co.KGaA, Berlin-Charlottenburg (DE)

(21) Appl. No.: 10/524,765

(22) PCT Filed: Jul. 30, 2003
(86) PCT No.: PCT/EP03/08393

§ 371 Date: Feb. 16, 2005

(30) Foreign Application Priority Data
Aug. 16, 2002 (DE) 102 38 434.7

(51) Int. Cl. A01H 1/00; C12Q 1/68; C07H 21/04; A01N 25/00; C12N 15/82; C12N 5/04

(52) U.S. Cl. 800/278; 504/116.1; 435/468; 435/419; 435/6; 536/23.6

(57) ABSTRACT

The present invention relates to a method of identifying herbicidally active compounds. The invention furthermore relates to nucleic acid constructs, to vectors comprising the nucleic acid constructs, to transgenic organisms and to their use. Moreover, the present invention relates to substances which have been identified using the abovementioned method.

The present invention relates to a method for identifying herbicidally active compounds. The invention furthermore relates to nucleic acid constructs, to vectors comprising the nucleic acid constructs, to transgenic organisms and to their use. Moreover, the present invention relates to substances which have been identified by the abovementioned method.

Modern agriculture without the use of herbicides is inconceivable. The value of the herbicides used worldwide is currently estimated at approx. 30 billion DM. Even though a large number of highly effective and ecologically acceptable herbicides are currently available, the need for novel herbicides results firstly from the fact that weeds keep developing a resistance to currently employed herbicides, which means that some of these can no longer be employed, and secondly from the fact that some of the herbicides are ecologically disadvantageous. Herbicides are currently in many cases still employed as mixtures which comprise several active ingredient components, which is ecologically not very advantageous and furthermore makes particular demands on the formulation.

Novel herbicides should be distinguished by as broad as possible a range of action, by ecological and toxicological acceptability and by low application rates.

The procedure so far for identifying and developing novel herbicides has been characterized by applying potential active ingredients directly to suitable test plants. The disadvantage of this procedure is that relatively large amounts of substance are necessary to carry out the tests. This is rarely the case in the age of combinatorial chemistry, where a very large variety of substances can be prepared, albeit in small amounts, and therefore constitutes an important limitation in the development of novel herbicides. Also, the direct application to the plants to be tested means that even the first screening step makes extremely high demands on the substance, since not only the inhibition or other modulation of the activity of a cellular target (as a rule a protein or enzyme) is required, but the substance must initially reach this target in the first place, which means that even this first step makes demands on the test substance with regard to the uptake by the plant, permeability through the various cell walls and membranes, persistence for achieving the desired effect, and, finally, inhibition/modification of the activity of the desired target enzyme.

In view of these demands, it is therefore not surprising that, on the one hand, the identification of novel active ingredients causes increasingly high costs and, on the other hand, the number of active ingredients which are discovered decreases all the time.

It was an object of the present invention to provide targets for identifying novel herbicides and to provide novel herbicides and their use. We have found that this object is achieved by a method of identifying herbicidally active substances wherein

a) the expression or the activity of the gene product of a nucleic acid or a gene encompassing:

aa) a nucleic acid sequence with the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51;
bb) a nucleic acid sequence which can be derived from the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 by backtranslation owing to the degeneracy of the genetic code;
cc) a nucleic acid sequence which is a derivative or a fragment of the nucleic acid sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 and which has at least 60% homology at the nucleic acid level;
dd) a nucleic acid sequence which encodes derivatives or fragments of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which have at least 50% homology at the amino acid level;
ee) a nucleic acid sequence which encodes a fragment or an epitope of a polypeptide which binds specifically to an antibody, the antibody specifically binding to a polypeptide which is encoded by the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51;
ff) a nucleic acid sequence which encodes a fragment of a nucleic acid shown in aa) and which has a translation releasing factor activity, a cobalamin synthase activity, an arginyl-tRNA synthase activity, an RNA helicase activity, a GTP binding protein activity, a pseudouridylate synthase activity, an adenylate kinase activity, a preprotein translocase secA precursor protein activity, a DCL protein activity, an arginine-tRNA ligase activity, a plastidial glutathione reductase activity, a transcription factor sigma activity, a calmodulin activity, an INT6 activity, a helicase YGL150c activity, an RNA-binding activity, a heat shock transcription factor activity, a chloro-plastidial DNA nucleoid binding activity or a Met2-type cytosine DNA methyltransferase activity; and/or
gg) a nucleic acid sequence which encodes derivatives of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which has at least 20% homology at the amino acid level and has an equivalent biological activity; or
   b) the expression or activity of an amino acid sequence which is encoded by a nucleic acid sequence of aa) to gg),
   is influenced and such substances which reduce or block the expression or the activity are selected.

“Expression” is understood as meaning the resynthesis in vitro and in vivo of nucleic acids and of proteins encoded by nucleic acids, in particular that of the abovementioned nucleic acid sequences and amino acid sequences. The term “expression” encompasses all biosynthetic steps which lead up to the mature protein or its catabolism, for example transcription, translation, modification or processing of nucleic acids and/or proteins, for example pre- or posttranscriptional processing steps or posttranslational modifications, for example splicing, editing, polyadenylation, capping, modifications of amino acids, for example glycosylation, methylation, acetylation, binding of coenzymes, phosphorylation, ubiquitation, binding of fatty acids, signal-peptide processing and the like.

For the purposes of the invention, “transcription” is to be understood as meaning RNA synthesis with the aid of an RNA polymerase in 5′-3′-direction using a DNA template. Translation is to be understood as meaning in-vitro and in-vivo protein biosynthesis. Gene product is understood as meaning any molecule and any substance which originates owing to the expression, for example the transcription or translation of a nucleic acid, for example of a DNA or RNA, for example of a gene, the term also encompassing the following processing products such as, for example, after splicing or modification. Thus, gene product is understood as meaning, for example, a processed RNA, for example a catalytic RNA such as a ribozyme, a functional RNA, such as tRNAs or rRNAs, or a coding RNA, such as mRNA. A protein, which is also understood as being a “gene product”, is synthesized as a consequence of the translation of an mRNA. Proteins can be subjected to various processing steps during and after translation, as enumerated above by way of example. “Activity of the gene product” is to be understood as meaning the biological activity or function of an RNA or of a protein, such as, for example, the enzymatic activity, the transporter activity, the regulatory activity, the property of binding receptors, the ability of binding certain proteins, nucleic acids or metabolites, for example in protein complexes, that is to say for example the regulatory property or the transporter function of the protein or of the RNA as it occurs naturally in the organism, to mention but a few. “Reduced activity of the gene product” is understood as meaning a reduction in the biological activity compared with the natural activity of the gene product by at least 10%, advantageously at least 20% or 30%, preferably at least 40%, 50% or 60%, especially preferably by at least 70%, 80% or 90% and very especially preferably by at least 95%, 96%, 97%, 98% or 99%. Blockage of the activity of the gene product means the complete, that is to say 100%, blockage of the activity or part-blockage of the activity, preferably an at least 80% or 90%, especially preferably at least 91%, 92%, 93%, 94% or 95%, very especially preferably at least 95%, 96%, 97%, 98% or 99% blockage of the biological activity.

The activity of the gene product can also be reduced indirectly, for example by inhibiting the formation or activity of interactants, for example by influencing the metabolic cascade in which the gene product plays a role. For example, an inhibition of not only the enzyme in question, but also of an enzyme or of a protein in the same metabolic cascade can take place, which leads to a blockage of the subsequent, preceding or any other enzyme involved and thus of the gene product described herein, for example by substrate or product inhibition. Such reductions by indirectly affecting the activity of an enzyme have been described extensively, for example, for the interaction of the glycolysis proteins and glycolysis metabolites and is readily applicable to other metabolic pathways in which the gene products described herein play a role. Equally, the activity of a gene product used in accordance with the invention can be reduced or inhibited by reducing or inhibiting the activity of interactants, for example other proteins, in a protein complex or in a substrate transport cascade with the gene product described herein. This may lead to the fact that the entire complex or the substrate transport is no longer activated or is not, or only incompletely, formed or can no longer be regulated. Examples of such influences on the activity have been described, for example, for spliceosomes, polymerases, ribosomes and the like.

“Fragment” is understood as meaning a part-sequence of a sequence described herein which encompasses fewer nucleotides or amino acids than the sequences described herein. For example, a fragment may encompass 1%, 5%, 10%, 30%, 50%, 70%, 90% of the original sequence. Preferably, a fragment encompasses 100, more preferably 50, even more preferably less than 20, amino acids of the corresponding nucleic acids.

The meaning of the individual biosynthesis steps is known to the skilled worker and can be found, for example, in “Molecular Biology of the cell”, Alberts, N.Y., 1998, “Biochemie” Stryer, 1988, New York, “Biochemieatlas”, Michal, Heidelberg, 1999 or in “Dictionary of Biotechnology”, Coombs, 1992.

Thus, one embodiment relates to a method according to the invention wherein the expression or the activity of the nucleic acids or amino acids mentioned is reduced or blocked by reducing or blocking the transcription, translation, processing and/or modification of at least one of the nucleic acid sequence or amino acid sequence according to the invention. In accordance with the invention, the activity of one, two, three or more sequences may be reduced or blocked.

The method according to the invention can be carried out in individual separate approaches or, advantageously, in a high-throughput screening and can be used for identifying herbicidally active substances or antagonists. Substances which interact with the abovementioned nucleic acids or their gene products can also be identified advantageously in the abovementioned method; these substances are potential herbicides whose action can be improved further by traditional chemical synthesis.

Substances identified, or selected, by the method can be applied advantageously to a plant in order to test the herbicidal activity of the substances. Those substances which show a herbicidal activity are selected. In a further advantageous embodiment of the method, the substances can also be identified in an in-vitro test, in addition to the abovementioned in-vivo test method. Such an in-vitro test with the nucleic acids according to the invention or their gene products has the advantage that the substances can be screened rapidly and in a simple fashion for their biological action. Such tests are also advantageously suitable for what is known as HTS.

The method can be carried out with free nucleic acids such as DNA or RNA, free gene products or, advantageously, in an organism, the organism used being eukaryotic or prokaryotic organisms, such as, advantageously, Gram-negative or Gram-positive bacteria, yeasts, fungi or, advantageously, plants such as monocotyledonous or dicotyledonous plants. The organisms used are, advantageously, the conditional or natural mutants relating to the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51. Conditional mutants are to be understood as being mutants which have to be induced first in order to show a reduction in expression, for example transcription or translation of the abovementioned nucleic acids or the gene products encoded by them. An example of such conditional mutants are mutants in which the nucleic acids are located downstream of a temperature-sensitive promoter which is nonfunctional at higher temperatures, that is to say which prevents transcription at higher temperatures, for example above 37° C. Also possible for example is the regulation of expression by an effector molecule, for example when the expression is controlled by a promoter which can be regulated, such as, for example, the promoter used in the Tet system (Gatz et al., Plant J. 2, 1992:397-404, tetracyclin-inducible) or the promoters described in EP-A-0 388 186 (benzenesulfonamide-inducible), EP-A-0 335 528 (abscisic-acid-inducible) or WO 93/21334 (ethanol- or cyclohexenol-inducible).

A further embodiment according to the invention is a method of identifying an antagonist of proteins which are encoded by a nucleic acid sequence as it is employed in the method according to the invention, in particular selected from the group consisting of:
a) a nucleic acid sequence with the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51;
b) a nucleic acid sequence which can be derived from the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 by backtranslation owing to the degeneracy of the genetic code;
c) a nucleic acid sequence which is a derivative or a fragment of the nucleic acid sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 and which has at least 60% homology at the nucleic acid level;
d) a nucleic acid sequence which encodes derivatives or fragments of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which have at least 50% homology at the amino acid level;
e) a nucleic acid sequence which encodes a fragment or an epitope of a polypeptide which binds specifically to an antibody, the antibody specifically binding to a polypeptide which is encoded by the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51;
f) a nucleic acid sequence which encodes a fragment of a nucleic acid shown in aa) and which has a translation releasing factor activity, a cobalamin synthase activity, an arginyl-tRNA synthase activity, an RNA helicase activity, a GTP binding protein activity, a pseudouridylate synthase activity, an adenylate kinase activity, a preprotein translocase secA precursor protein activity, a DCL protein activity, an arginine-tRNA ligase activity, a plastidial glutathione reductase activity, a transcription factor sigma activity, a calmodulin activity, an INT6 activity, a helicase YGL150c activity, an RNA-binding activity, a heat shock transcription factor activity, a chloroplastidial DNA nucleoid binding activity or a Met2-type cytosine DNA methyltransferase activity; and/or
g) a nucleic acid sequence which encodes derivatives of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which has at least 20% homology at the amino acid level and has an equivalent biological activity;
   by following through the following method steps
i) contacting cells which express the protein, or the protein, with a candidate substance;
ii) testing the biological activity of the protein;
iii) comparing the biological activity of the protein with a standard activity in the absence of the candidate substance, a reduced biological activity of the protein indicating that the candidate substance is an antagonist.
   ii) describes the testing of one of the above-described biological activities, for example an enzyme activity as it is shown in the examples, or a binding, preferably a strong binding between protein material and candidate substance.

In an advantageous embodiment of the above-described method, the antagonist(s) identified under iii) is/are applied to a plant to test its/their herbicidal activity and the antagonist(s) which show(s) herbicidal activity is/are selected.

The method according to the invention can be carried out in individual separate approaches in vivo or in vitro and/or advantageously jointly or, especially advantageously, in a high-throughput screening and can be used for identifying herbicidally active substances or antagonists.

The nucleic acid sequences identified or selected in the method according to the invention are essential for the growth and the development of higher plants. Suppression of the formation of the gene products, i.e. of expression, for example by exerting a specific effect on, for example, the transcription, the translation or the processing and/or of the suppression of the function or biological activity exerted by the encoded gene products in intact plants by substances, advantageously low-molecular-weight substances with a molecular weight of less than 1000 daltons, advantageously less than 900 daltons, preferably less than 800 daltons, particularly preferably less than 700 daltons, very particularly preferably less than 600 daltons, advantageously with a Ki value of less than 10⁻⁷, advantageously less than 10⁻⁸, preferably less than 10⁻⁹ M, advantageously this inhibitory effect should be attributable to a specific inhibition of the biological activity of the nucleic acids according to the invention and/or of the proteins encoded by these nucleic acids, i.e. no inhibition by these low-molecular-weight substances of further, closely related nucleic acids and/or of the proteins encoded by these nucleic acids should take place. Moreover, the low-molecular-weight substances should advantageously have a molecular weight of greater than 50 daltons, preferably greater than 100 daltons, especially preferably greater than 150 daltons, very especially preferably greater than 200 daltons. Preferably the low-molecular-weight substances should have fewer than three hydroxyl groups on a carbon atom-containing ring. Furthermore, the molecule should also not comprise (a) free acid or lactone group(s) and no phosphate group and not more than one amino group in the molecule. Bases such as adenosine in the molecule are also less preferred. The substances, advantageously the low-molecular-weight substances, but also proteinogenic substances or sense or antisense RNA or antibodies or antibody fragments identified via the method according to the invention advantageously lead, by virtue of their inhibitory effects, to massive changes regarding the growth and the development of the plants treated or in question. The substances identified in the method according to the invention are therefore suitable as herbicides in agriculture.

The nucleic acids SEQ ID NO: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 used in the method according to the invention are essential for organisms, preferably for plants. Their disruption, or the blockage of their expression, halts the development of plants at an early developmental stage. The gene products of the abovementioned sequences can be found for example in the polypeptides of the sequences SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52.

SEQ ID NO: 1, whose expression is blocked in line 303317, encodes a protein (F2809.40) which has similarities with the Synechocystis sp. translation releasing factor RF-2 (PIR:S76448) and which is located on the Arabidopsis chromosome 3 (BAC ATF2809, Accession AL137080). Moreover, the protein has the araC family signature.

SEQ ID NO: 3, whose expression is blocked in line 304149 encodes a cobalamin synthesis protein (MSH 12.9) which is located on the Arabidopsis chromosome 5 (P1 clone MSH12, Accession AB006704).

SEQ ID NO: 5, whose expression is blocked in line 120701, encodes an ORF (T25K17.110) on chromosome 4 (BAC ATT25K17, Accession AL049171), which possibly encodes an arginyl-tRNA synthetase. This ORF comprises the EST: gb:AA404880, T76307.

SEQ ID NO: 7, whose expression is blocked in line 126548 and which is located on chromosome 4 of the Arabidopsis genome (BAC ATF17A8, Accession AL049482), encodes a putative protein (F17A8.80) with similarity to a murine RNA helicase (Mus musculus, PIR2:I84741).

SEQ ID NO: 9, whose expression is blocked in line 127023, encodes a putative protein (AT4g39780) which is located on chromosome 4 (BAC ATT19P19, Accession number AL022605) and which has homologies with the Arabidopsis thaliana protein RAP 2.4, which comprises the AP2 domain. Moreover, the ORF comprises the ESTs gb:T46584 and AA394543.

SEQ ID NO: 11, whose expression is blocked in line 127235, encodes the ORF F9K20.4, which is located on the Arabidopsis chromosome 1 (BAC F9K20, Accession AC005679). This ORF F9K20.4 encodes a putative protein with similarity to gi|1786244 a hypothetical 24.9 kD protein in the surA-hepA intergenic region yab0 of the Escherichia coli genome and to gb|AE000116, a hypothetical protein of the YABO family PF|00849. Furthermore, the protein encoded by ORF F9K20.4 has a conserved pseudouridylate synthase domain, which is involved in the modification of uracil in RNA molecules. Accordingly, the ORF F9K20.4 shows significant homology with various pseudouridylate synthases in the blastp alignment under standard conditions.

SEQ ID NO: 13, whose expression is blocked in line 218031, encodes a putative adenylate kinase (At2g37250). The ORF At2g37250 is located on chromosome 2 of clone F3G5 (Accession AC005896) of Arabidopsis.

The putative protein (ORF T29H11_{—}270, Accession AL049659) which is encoded by SEQ ID NO: 15 and whose expression is blocked in line 171042 shows similarity with the pol polyprotein of the Equine Infectious Anemia Virus (PIR:GNLJEV). The sequence is located on chromosome 3 of the BAC clone T29H11 of Arabidopsis.

SEQ ID NO: 17, whose expression is blocked in line KO_T3_{—}02-33338-3, is located on chromosome 5 of the P1 clone MJE7 (Accession AB020745). The sequence encodes ORF MEJ7.11. ORF MEJ7.11 is an unknown protein.

SEQ ID NO: 19, whose expression is blocked in line KO_T3_{—}02-33885-2 encodes an unknown protein (=ORF F14G9.26). The ORF is located on chromosome 1 of the BAC clone F14G8 with Accession AC069159.

SEQ ID NO: 21, whose expression is blocked in line KO_T3_{—}02-35172-2, encodes an unknown protein. The ORF MAB16.6 only has homologies with other unknown proteins. The sequence is located on chromosome 5 of the P1 clone MAB16 with Accession AB018112.

SEQ ID NO: 23, whose expression is blocked in line 305861, encodes a preprotein translocase secA precursor protein, therefore a chloroplastidial SecA protein for the transport of proteins via the thylakoid membrane. This ORF, with Accession T7B11.6, AC007138, can be found on the BAC clone T7B11 of chromosome 4.

The protein encoded by SEQ ID NO: 25 (=line 303814), with Accession F2G19.1, which has significant homology with the tomato DCL protein (PIR: S71749) is located on the BAC clone F2G19, Accession Number AC083835, chromosome 1.

SEQ ID NO: 27 (=line KO-T3-02-13224-1) encodes an arginine-tRNA ligase with Accession T25K17.110. This ORF is located on the BAC clone T25K17 with Accession Number AL049171 and thus on chromosome 4.

SEQ ID NO: 29 (=line KO-T3-02-15114-2) encodes a plastidial glutathione reductase. This ORF is annotated on the BAC clone T5N23 with Accession T5N23.20, Accession Number AL138650 on chromosome 3.

SEQ ID NO: 31 (=line KO-T3-02-18601-1) encodes a transcription initiation factor Sigma homolog. This ORF with Accession F22O13.2 is annotated on the BAC clone T22O13, Accession Number AC003981, on chromosome 1.

SEQ ID NO: 33 (=line 304143) encodes a putative calmodulin-like protein. This ORF, with Accession At2g15680, is annotated on the BAC clone F9013 with the Accession Number AC006248 on chromosome 2.

The unknown ORF MPX5.1, which is encoded by SEQ ID NO: 35 (=line KO-T3-02-40322-2), is annotated on the BAC clone MPX5, Accession Number AP002048, on chromosome 3.

SEQ ID NO: 37 (=line KO-T3-02-40309-1) encodes a protein with great similarity to INT6, a breast-cancer associated protein, and with similarity to an “initiation factor 3” protein. This ORF with Accession F28O9.140 is annotated on the BAC clone F28O9, Accession Number AL137080, on chromosome 3.

The protein encoded by SEQ ID NO: 39 (=line KO-T3-02-40309-1) has great similarity with the Saccharomyces DNA helicase YGL150c. This ORF with the Accession F28O9.150 is located on the BAC clone F28O9, Accession Number AL137080, on chromosome 3.

SEQ ID NO: 41 (=line KO-T4-02-006664) encodes a protein with similarity to an RNA-binding protein. This ORF with the Accession MKN22.2 is located on the BAC clone MKN22, Accession Nummer AB019234, of chromosome 5.

SEQ ID NO: 43 (=line KO-T4-02-00666-4) encodes an unknown protein. This ORF with the Accession MEE6.19 is annotated on the BAC clone MEE6, Accession Number AB010072, on chromosome 5.

SEQ ID NO: 45 (=line KO-T3-02-41568-2) encodes a putative heat-shock transcription factor. This ORF with the Accession At2g26150 is located on the BAC clone T19L18, Accession Number AC004747, on chromosome 2.

The ORF At2g28030, which is shown in SEQ ID NO: 47 (=line KO-T3-02-42903-1) encodes a putative chloroplastidial protein which binds to the DNA nucleoid. This ORF At2g28030 is annotated on the BAC clone T1E2, Accession Number AC006929, on chromosome 2.

SEQ ID NO: 49 (=line KO-T3-0241395-1) encodes a protein with similarity to a putative Met2-type cystosine DNA methyltransferase and has great similarity with a Arabidopsis thaliana DNA-(cystosine-5)-methyltransferase. This ORF with Accession AT4g08990 is annotated on the BAC clone ATCHRIV25, Accession Number AL161513, on chromosome 4.

SEQ ID NO: 51 (=line KO-T3-02-44634-4) encodes a protein with great similarity to a postulated Arabidopsis thaliana protein. This ORF with Accession F12B17_{—}70 is located on the BAC clone F12B17, Accession Number AL353995, on chromosome 5. All of the abovementioned sequences were identified in Arabidopsis.

The suppression of the formation of the gene products or the suppression of the function or activity exerted by the encoded gene products in intact plants by a low-molecular-weight substance leads to reduced, preferably to suppressed growth; the development of the plant is drastically altered and suppressed. They are therefore advantageously suitable for identifying herbicides.

The abovementioned sequences or functional portions thereof make possible the identification of herbicides which can be used in agriculture, for example, via a method which comprises the following steps:
a) providing two lines of an organism which functionally express the gene products encoded by one of the sequences described for the method according to the invention, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 or by the above-described derivatives or fragments thereof which have the biological activity of these sequences, the expression level of the lines being different, for example by mutagenesis of one line and identification of a mutant with increased or reduced expression and/or activity of the abovementioned gene product in comparison with the starting line or, for example, by generating recombinant organisms, advantageously transgenic plants, plant tissues such as tissues of, for example, leaf, root, shoot or stem, plant seeds, plant calli or plant cells which functionally express the sequences described in accordance with the invention, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 oder SEQ ID NO: 51 or derivatives or fragments thereof which have the biological activity of these sequences;
b) addition of chemical compounds (which are to be tested for their herbidical activity) to the lines with the different expression or activity levels of the gene product, for example to recombinant organisms mentioned under a) and nonrecombinant starting organisms with a different, preferably lower, expression or activity level of the gene product;
c) determination of the biological activity, for example the enzymatic activity, the growth or the vitality of the two lines, for example of the recombinant organisms, in comparison with the nonrecombinant starting organisms after addition of chemical compounds in accordance with item b); and
d) selection of the chemical compounds which reduce or completely inhibit or block the biological activity, for example the enzymatic activity, the growth or the vitality of the line with the lower activity, for example which reduce or completely inhibit or block the biological activity, the growth or the vitality of the nonrecombinant organisms, of the chemical compounds determined in accordance with item c), in comparison with the treated recombinant organisms.

A herbicide which can be used in agriculture can also be identified when the recombinant organisms generated above in
a) are tested in a method comprising the following steps:
(b) addition of chemical compounds to be tested for their herbicidal activity to the recombinant organisms mentioned under (a); and
(c) determination of the biological activity, for example of the enzymatic activity, the growth or the vitality of the recombinant organisms after addition of chemical compounds in accordance with (b) in comparison with the same untreated recombinant organisms; and
(d) selection of the chemical compound which reduces or completely inhibits or blocks the biological activity, for example the enzymatic activity, the growth or the vitality of the treated organisms in comparison with the untreated organisms.

Chemical compounds which reduce the biological activity, the growth or the vitality of the organisms are understood as meaning compounds which inhibit, i.e. reduce or block, the biological activity, the growth or the vitality of the organisms by at least 10%, 20% or 30%, advantageously by at least 40%, 50% or 60%, preferably by at least 70%, 80 or 90%, especially by at least 91%, 92%, 93%, 94% or 95%, very especially preferably by at least 96%, 97%, 98% or 99%.

An advantageous substance is in particular a substance which damages the cell lines with lower activity or, preferably, which is lethal but which does not damage, or is not lethal for, cell lines which have a higher activity of the gene product.

In general, lines of organisms can be employed in the abovementioned method which express the sequences according to the invention and in particular the gene products which are encoded by nucleic acids according to the invention, but which are not recombinant, as long as one line shows higher gene expression or activity of the gene product than another line. Such lines can occur naturally or be generated by mutageneses.

Assay systems which allow the identification of substances which suppress the formation of the gene products and/or the functions exerted by the gene products or the activity of the gene products in intact plants, plant parts, plant tissues or plant cells are known to the skilled worker. Examples which may be referred to here are test hydrophobic interactions with the chip. The ligands are subsequently applied to the chip prepared in this way, for example using an autosampler. After one or more wash steps with buffers of various ionic strengths, the bound ligands are analyzed using the LDI laser. In doing this, the binding strength of the ligands is determined after each washing step.

A further advantageous detection method that may be mentioned is what is known as the Biacore method, where the refraction index at the surface upon binding of ligands and the protein bound to the surface is analyzed. In this method, a collection of small ligands is added sequentially to a measuring cell with the bound protein. The binding at the surface is determined by an increase in what is known as plasmon resonance (=SPR) by recording the laser refraction from the surface. In general, the change in refraction index which is determined for a change in the mass concentration at the surface, is equal for all proteins or polypeptides, that is to say this method can be used advantageously for a very wide range of proteins (Liedberg et al., Sens. Actuators, 1984, 4, 299-304). Again, as described above, recombinantly expressed proteins are used advantageously, and these proteins are bound to the Biacore chip (Uppsala, Sweden), for example via histidine residues (for example his-tag). The chip prepared in this way is again contacted with the ligands, for example with an autosampler, and the binding is measured via a detection system available from Biacore with the aid of the SPR signal, i.e. via the change in the refraction index.

The methods according to the invention have a series of advantages such as, for example:
novel potential targets for herbicidal active ingredients can be identified,
identification of herbicides which have as complete an action as possible, independently of the plant species,
substances which were generated by means of combinatorial chemistry and which can be distinguished by a great variety, but by low amounts which are available, can be tested efficiently for inhibitors of the newly identified targets
in the case of herbicides which, for example, have a very broad activity (nonselective herbicides or else selective herbicides), they permit resistance to these herbicides to be mediated to agriculturally useful plants (see description hereinbelow).

For example, substances which bind particularly specifically to, for example, a protein or protein fragment encoded by a nucleic acid whose expression is essential for the growth of the plants can be isolated using the abovementioned methods. This makes systems for the inhibition of enzymes such as adenylate kinase as described by Skoblov et al. (FEBS Letters, 395 (2-3), 1996: 283-285), by Russel et al. (J. Enzyme Inhib., 9 (3),1995:179-194 and), Wiesmüller et al. (FEBS Letters, 363, 1995: 22-24) or Schlattner et al. (Phytochemistry, 42, 1996: 589-594). For example, such test systems can be used advantageously for what are known as inhibition assays for the gene product identified in line 218031, for example.

Further advantageous assay systems are, for example, fluorescence correlation spectroscopy (=FCS). With the aid of FCS (Brock et al., PNAS, 1999, 96, 10123-10128; Lamb et al., J. Phys. Org. Chem., 2000, 13654-658), it is possible to measure the diffusion of molecules over time, or to determine the difference of the bound versus free molecules. To this end, the molecules to be studied are fluorescence-labeled and, for example, a defined volume is placed into microtiter plates. The fluctuation of the molecules in the samples is driven by the Brownian movement. The translateral or rotational diffusion and conformation changes of the molecules can be monitored by a laser focussed into the sample and analyzed via a correlation. Owing to binding to other substances, the diffusion coefficient of the molecules changes. The binding of the molecules can be determined or quantified with the aid of various algorithms via the change in the diffusion coefficient. This method allows advantageous measurements to be carried out within a wide concentration range. The method is advantageously suitable for measuring recombinant proteins which are advantageously provided with what is known as a his-tag to facilitate purification via commercially available chromatography columns (Porath et al., Nature 1975, 258, 598-599). The protein purified in this way is finally provided with a fluorescence marker such as, for example, carboxytetramethylrhodamine or BODIPY® (for example, BODIPY 576/589 Angiotensin II, NEN® Life Science Products, Boston, Mass., USA). An excess of the compound or substance to be tested is subsequently added to the protein. The diffusion of the protein labeled in this way is finally determined using an FCS system (for example, ConfoCor2 with LSM 510, Carl Zeiss microscope, Jena, Germany).

A further advantageous detection method for the method according to the invention is what is known as the surface-enhanced laser desorption ionization method (=SELDI ProteinChip®). This method was first described by Hutchens and Yip (1980). Using this method, which was developed for the reproducible simultaneous identification of biomarkers or antigens (Hutchens and Yip, Rapid Commun. Mass Spectrom, 1993, 7, 576-580), the ligand-protein binding can be analyzed via mass spectrometry. Detection is via normal TOF detection (=time of flight). This method too allows recombinantly expressed proteins to be expressed and purified as described above. To carry out the measurement, the protein is immobilized on the SELDI ProteinChips®, for example via the his-tags which have already been used for purification or via ion interactions or growth of the plants can be isolated using the abovementioned methods. This makes possible a simplified identification of possible inhibitors which inhibit proteins, for example in their enzyme properties, binding properties or other activities, for example also by inhibiting their processing, as described above, or which inhibit their transport within the cell or their import or export from organelles or cells. The substances identified in this way can also be applied to plants in a further step in screening methods as are known to the skilled worker and studied for their effect on the growth and the development. Thus, a selection is made from the infinite number of chemical compounds which would be suitable for a screening method, which selection makes it considerably easier for the skilled worker to identify herbicidal substances.

“Specific binding” is understood as meaning the specificity of interactions between two partners, for example proteins among themselves or between protein (enzyme) and substrate (substrate specificity). It is based on a specific molecular spatial structure. The destruction of this structure is termed denaturation, which is frequently irreversible, in most cases leading to loss of specificity. This biological activity depends greatly on the environmental conditions (buffer, temperature, contacts with nonphysiological surfaces like glass, or lack of cofactors). Enzyme-substrate or cofactor bindings, receptor-ligand bindings or antibody-antigen bindings are termed specific types of binding. In the simplest case, the enzyme-substrate interaction is described thermodynamically using the Michaelis-Menten equation. It describes the enzyme activity beyond what is known as the Michaelis-Menten constant, which, in turn, reflects the kinetics. This constant is also the unit of measurement for the enzyme activity which, in turn, reflects the specificity. Definition of the enzyme activity unit (in accordance with IUB): one unit U corresponds to the amount of enzyme which catalyzes the conversion of one micromole of substrate per minute under precisely defined experimental conditions. The specific activity is usually given in U/mg.

In a further step, the identified substances can then be applied to plants, microorganisms or cells, for example to plant cells, and the effect which they have on the metabolism of these plants can then be observed, for example enzyme activities, photosynthesis activities, metabolic activity, fixation rate, gas exchange, DNA synthesis, growth rates. These methods and many others which are known to the skilled worker are suitable for studying the viability of cells. Substances which reduce, in particular block, the growth of, for example cells, in particular plant cells, are then preferably suitable as a choice for herbicidal compositions.

Furthermore, studies into the application rates of the herbicides which have been found can be made at a very early stage. Moreover, the high specificity for, and efficacy against, weeds can be determined readily.

A multiplicity of chemical compounds can be tested rapidly and in a simple manner for herbicidal properties with the method according to the invention. The method allows a reproducible selection from a large number of substances of specifically those which are highly effective to subsequently carry out, on these substances, further in-depth tests which are familiar to the skilled worker.

The invention furthermore relates to a method of identifying inhibitors of plant proteins, which inhibitors have a potentially herbicidal action and which are encoded by the nucleic acid sequences used in the method according to the invention, by cloning the gene products, overexpressing them in a suitable expression cassette—for example in insect cells—disrupting the cells and employing the cell extract directly or after concentration or isolation of the protein in an assay system for measuring the biological activity in the presence of low-molecular-weight chemical compounds.

The invention therefore furthermore relates to substances identified by the methods according to the invention, the substances advantageously being low-molecular-weight substances with a molecular weight of less than 1000 daltons, advantageously less than 900 daltons, preferably less than 800 daltons, especially preferably less than 700 daltons, very especially preferably less than 600 daltons, advantageously with a Ki value of less than 10⁻⁷, advantageously less than 10⁻⁸, preferably less than 10⁻⁹ M. Advantageously, this inhibitory effect should be attributable to a specific inhibition of the biological activity of the nucleic acids according to the invention and/or of the proteins encoded by these nucleic acids, i.e. no inhibition by these low-molecular-weight substances of further closely related nucleic acids and/or of the proteins encoded by these nucleic acids should take place. Furthermore, the preferred low-molecular-weight substances should advantageously have a molecular weight greater than 50 daltons, preferably greater than 100 daltons, especially preferably greater than 150 daltons, very especially preferably greater than 200 daltons. The low-molecular-weight substances should advantageously have less than three hydroxyl groups on a carbon-atom-containing ring. Furthermore, no free acid or lactone group(s) and no phosphate group and not more than one amino group should be present in the molecule. Also, bases such as adenosine are less preferred in the molecule.

In an advantageous embodiment of the substances, the substance is a proteinogenic substance, an antisense RNA, an inhibitory or an interfering RNA (RNAi).

The term “sense” refers to the strand of a double-stranded DNA which is homologous to the mRNA transcript. The “antisense” strand contains an inverted sequence which is complementary to that of the “sense” strand. For example, an antisense nucleic acid molecule comprises a nucleotide sequence which is complementary to the “sense” nucleic acid molecule which encodes a protein or an active RNA, for example complementary to the coding strand of a double-stranded cDNA molecule or complementary to an mRNA sequence. As a consequence, an antisense nucleic acid molecule can form hydrogen bonds with a sense nucleic acid molecule. The antisense nucleic acid molecule can be complementary to any of the coding strands shown here or only to part thereof. The term “coding region” refers to the region of a nucleic acid sequence whose codons are translated into amino acids. Also, the antisense nucleic acid molecule can be complementary to “noncoding regions” of the coding strand of the nucleic acid molecules shown. The term “noncoding regions” refers to 5′- and 3′-sequences which flank the coding region and which are not translated into a polypeptide (for example also termed 5′- and 3′-untranslated regions). The nucleic acid molecule which encompasses an antisense sequence can also encompass further elements which are important for the expression and stability of the molecule, for example capping structures, poly-A-tails and the like.

The antisense nucleic acid molecule can be complementary to the entire coding region of an mRNA, but it can also be an oligonucleotide which is complementary to only part of the coding or noncoding region of the mRNA. For example, an antisense oligonucleotide can be complementary to the region which encompasses or surrounds the translation start of the mRNA. For example, an antisense oligonucleotide can advantageously have a length of 10, 15, 20, 25, 30, 35, 40, 45 or 50 nucleotides. An antisense nucleic acid molecule can be generated by chemical synthesis and enzymatic ligation by methods known to the skilled worker. An antisense nucleic acid molecule can be synthesized chemically using naturally occurring nucleotides or nucleotides which have been modified in various ways, so that the biological stability of the molecules is increased or the physical stability of the duplex which forms between the antisense and sense nucleic acid is increased; for example, phosphorothioate derivatives and acridine-substituted nucleotides can be used. Examples of modified nucleotides which can be used for the generation of antisense nucleic acids encompass 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl)uracil, (acp3)w, and 2,6-diaminopurine.

As an alternative, antisense nucleic acid molecules can be prepared biologically using expression vectors into which polynucleotides with the opposite orientation have been cloned (so that RNA transcribed from the inserted polynucleotide is in antisense orientation relative to a target polynucleotide as has been described further above).

The antisense nucleic acid molecule can also be an “α-anomeric” nucleic acid molecule. An “α-anomeric” nucleic acid molecule forms specific double-strand hybrids with complementary RNAs in which the strands run in parallel with each other, in contrast to ordinary β units. The antisense nucleic acid molecule can encompass 2-0-methylribonucleotides or chimeric RNA-DNA-analogs.

Moreover, the antisense nucleic acid molecule can be a ribozyme. Ribozymes are catalytic RNA molecules with a ribonuclease activity which are capable of cleaving single-stranded nucleic acids, such as, for example, mRNA, to which they have a complementary region. Ribozymes (for example hammerhead ribozymes) can be used for catalytically or noncatalytically cleaving mRNA of the sequences described herein, thus preventing translation of the mRNA. A ribozyme which is specific for one of the nucleic acid sequences mentioned herein can be constructed on the basis of the cDNA sequences shown herein or on the basis of heterologous sequences which can be identified by the methods described herein. For example, a derivative of the Tetrahymena L-19 IVSRNA can be prepared in which the nucleotide sequence of the active region is complementary to the nucleotide sequence which is cleaved in a coding mRNA. As an alternative, one of the coding or noncoding sequences described herein or of an mRNA thereof may also be used in order to select a catalytic RNA from an RNA pool (see, for example, Bartel, 1993, Science, 261, 1411). As an alternative, the expression can also be inhibited by nucleotide sequences which are complementary to a regulatory region of the nucleic acid sequences described herein (for example a promoter or enhancer) forming a triple-helical structure, which prevents transcription of the subsequent gene (for example Helene, 1991, Anticancer-Drug Des. 6, 596; Helene, 1992, Ann. NY Acad. Sci. 660, 27, or Maher, 1992, Bioassays, 14, 807).

The dsRNAi method (=“double-stranded RNA interference”) has been described repeatedly in animal and plant organisms (for example Matzke M A et al. (2000) Plant Mol Biol 43:401-415; Fire A. et al (1998) Nature 391:806-811; WO 99/32619; WO 99/53050; WO 00/68374; WO 00/44914; WO 00/44895; WO 00/49035; WO 00/63364). The processes and methods described in the references are expressly referred to. Efficient gene suppression can also be demonstrated in the case of transient expression or following transient transformation, for example as a consequence of a biolistic transformation (Schweizer P et al. (2000) Plant J 2000 24: 895-903). dsRNAi methods are based on the phenomenon that highly efficient suppression of the expression of the gene in question is brought about by the simultaneous introduction of complementary strand and counterstrand of a gene transcript. The phenotype generated is very similar to a corresponding knock-out mutant (Waterhouse P M et al. (1998) Proc Natl Acad Sci USA 95:13959-64).

The dsRNAi method can be used advantageously for reducing the expression of the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51, their derivatives and fragments. As described inter alia in WO 99/32619, dsRNAi approaches are markedly superior to traditional antisense approaches.

The invention therefore furthermore relates to double-stranded RNA molecules (dsRNA molecules) which, when introduced into an organism, advantageously a plant (or a cell, tissue, organ or seed derived therefrom), bring about the reduction of the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51, their derivatives or fragments or of the proteins encoded by them. In the double-stranded RNA molecule for reducing the expression of a protein which is encoded by the sequences SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52,
i) one of the two RNA strands is essentially identical to at least a part of a nucleic acid sequence with the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51, and
ii) the respective other RNA strand is essentially identical to at least a part of the complementary strand of one of the nucleic acid sequences mentioned under (i).

“Essentially identical” means that the dsRNA sequence may also display insertions, deletions and individual point mutations in comparison with the target sequence (SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51) while still efficiently bringing about reduced expression. Preferably, the homology according to the above definition amounts to at least 75%, preferably at least 80%, very especially preferably at least 90%, most preferably 100%, between the sense strand of an inhibitory dsRNA and a subsection of a nucleic acid sequence with the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 (or between the antisense strand of the complementary strand of a nucleic acid of the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51, respectively). The length of the subsection amounts to at least 10 bases, preferably at least 25 bases, especially preferably at least 50 bases, very especially preferably at least 100 bases, most preferably at least 200 bases or at least 300 bases. As an alternative, an “essentially identical” dsRNA can also be defined as a nucleic acid sequence which is capable of hybridizing with a part of a gene transcript of the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 (for example in 400 mM NaCl, 40 mM PIPES pH 6.4, 1 mM EDTA at 50° C. or 70° C. for 12 to 16 hours).

The dsRNA may consist of one or more strands of polymerized ribonucleotides. Modifications both of the sugar-phosphate backbone and of the nucleosides may furthermore be present. For example, the phosphodiester bonds of the natural RNA can be modified in such a way that they comprise at least one nitrogen or sulfur heteroatom. Bases can be modified in such a way that the activity of, for example, adenosine deaminase is limited. Those and further modifications are described hereinbelow in the methods for stabilizing antisense RNA.

The dsRNA can be generated enzymatically or synthesized chemically, either fully or in part.

The double-stranded structure can be formed starting from a single, autocomplementary strand or starting from two complementary strands. In a single, autocomplementary strand, sense and antisense sequence can be linked by a linking sequence (linker) and form, for example, a hairpin structure. The linking sequence can preferably be an intron, which is spliced out once the dsRNA has been synthesized. The nucleic acid sequence encoding a dsRNA can comprise further elements, such as, for example, transcription termination signals or polyadenylation signals. If the two strands of the dsRNA are to be combined in a cell or an organism, advantageously in a plant, this can be done in various ways:
a) transformation of the cell or the organism, advantageously a plant, with a vector comprising both expression cassettes,
b) cotransformation of the cell or the organism, advantageously a plant, with two vectors, where one of them comprises the expression cassettes with the sense strand, while the other comprises the expression cassettes with the antisense strand,
c) hybridization of two organisms, advantageously plants, each of which has been transformed with a vector, one vector comprising the expression cassettes with the sense strand while the other comprises the expression cassettes with the antisense strand.

The formation of the RNA duplex can be initiated either outside the cell or within same. As in WO 99/53050, the dsRNA may also comprise a hairpin structure by linking sense and antisense strands by a linker (for example an intron). The autocomplementary dsRNA structures are preferred since they only require the expression of one construct and always comprise the complementary strands in an equimolar ratio.

Expression cassettes encoding the antisense or sense strand of a dsRNA or the autocomplementary strand of the dsRNA are preferably inserted into a vector and, using the methods described hereinbelow, stably inserted into the genome of a plant (for example using selection markers) to ensure permanent expression of the dsRNA.

The dsRNA can be introduced using an amount which makes possible at least one copy per cell. Higher amounts (for example at least 5, 10, 100, 500 or 1000 copies per cell) may bring about more efficient reduction.

As already described, 100% sequence identity between dsRNA and a gene transcript of the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 is not necessarily required in order to bring about an efficient reduction of the expression of the sequences mentioned. Accordingly, there is an advantage in as far as that the method is tolerant to sequence deviations as may be present as the result of genetic mutations, polymorphisms or evolutionary divergences. Using the dsRNA which has been generated starting from the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 of one organism, it is thus possible, for example, to suppress the expression of the sequences in another organism. The high degree of sequence homology between the sequences from different organisms suggests a high degree of conservation of these proteins within, for example, plants, so that the expression of a dsRNA derived from one of the disclosed sequences as shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 is also likely to have an advantageous effect in other plant species.

The dsRNA can be synthesized either in vivo or in vitro. To this end, a DNA sequence encoding a dsRNA can be introduced into an expression cassette under the control of at least one genetic control element (such as, for example, promoter, enhancer, silencer, splice donor or splice acceptor, polyadenylation signal). Suitably advantageous constructions are described further below. Polyadenylation is not required, nor is it necessary for translation initiation elements to be present.

A dsRNA can be synthesized chemically or enzymatically. Cellular RNA polymerases or bacteriophage RNA polymerases (such as, for example, T3, T7 or SP6 RNA polymerase) may be used for this purpose. Suitable methods for the in vitro expression of RNA are described (WO 97/32016; U.S. Pat. No. 5,593,874; U.S. Pat. No. 5,698,425, U.S. Pat. No. 5,712,135, U.S. Pat. No. 5,789,214, U.S. Pat. No. 5,804,693). Prior to introduction into a cell, tissue or organism, dsRNA which has been synthesized chemically or enzymatically in vitro can be isolated from the reaction mixture in various degrees of purity, for example by extraction, precipitation, electrophoresis, chromatography or combinations of these methods. The dsRNA can be introduced directly into the cell or else applied extracellularly (for example into the interstitial space).

“Antibodies” are understood as meaning, for example, polyclonal, monoclonal, human or humanized or recombinant antibodies or fragments thereof, single-chain antibodies or else synthetic antibodies. Antibodies according to the invention or fragments thereof are understood as meaning, in principle, all classes of immunoglobulins such as IgM, IgG, IgD, IgE, IgA or their subclasses such as the subclasses of IgG or their mixtures. Preferred are IgG and its subclasses such as, for example, IgG₁, IgG₂, IgG₂ₐ, IgG_{2b}, IgG₃ or IgG_{M}. Especially preferred are the IgG subtypes IgG₁ or IgG_{2b}. Fragments which may be mentioned are all truncated or modified antibody fragments with one or two binding sites which are complementary to the antigen, such as antibody portions with a binding site formed by light and heavy chain which corresponds to the antibody, such as Fv, Fab or F(ab′)₂ fragments or single-strand fragments. Preferred are truncated double-strand fragments such as Fv, Fab or F(ab′)₂. These fragments can be obtained, for example, via the enzymatic route by cleaving off the Fc portion of the antibodies using enzymes such as papain or pepsine, by chemical oxidation or by genetic manipulation of the antibody genes. Genetically engineered nontruncated fragments may also be used advantageously. The antibodies or fragments can be used alone or in mixtures. Antibodies can also be part of a fusion protein.

The substances identified can be chemically synthesized or microbiologically produced substances which may be found, for example, in cell extracts of, for example, plants, animals or microorganisms. Furthermore, while the substances mentioned may be known in the prior art, they may not be known as yet as herbicides. The reaction mixture can be a cell-free extract or encompass a cell or cell culture. Suitable methods are known to the skilled worker and are described generally, for example, in Alberts, Molecular Biology the cell, 3rd Edition (1994), for example chapter 17. The substances mentioned may, for example, be added to the reaction mixture or the culture medium or injected into the cells or sprayed onto a plant.

Once a sample comprising an active substance according to the method according to the invention has been identified, it is either possible to isolate the substance directly from the original sample, or the sample can be divided into different groups, for example when it is composed of a multiplicity of different components, in order to thus reduce the number of the different substances per sample and then to repeat the method according to the invention with such a “subsample” of the original sample. Depending on the complexity of the sample, the above-described steps can be repeated several times, preferably until the sample identified in accordance with the method according to the invention only encompasses a small number of substances or just one substance. Preferably, the substance identified in accordance with the method according to the invention, or derivatives of the substance, are formulated further so that it is suitable for use in plant breeding or in plant cell or tissue culture.

The substances which were tested and identified in accordance with the method according to the invention can be, for example: expression libraries, for example cDNA expression libraries, peptides, proteins, nucleic acids, antibodies, small organic substances, hormones, PNAs or similar (Milner, Nature Medicin 1 (1995), 879-880; Hupp, Cell. 83 (1995), 237-245; Gibbs, Cell. 79 (1994), 193-198 and references cited therein). These substances can also be functional derivatives or analogs of the known inhibitors or activators. Methods for the preparation of chemical derivatives or analogs are known to the skilled worker. The abovementioned derivatives and analogs can be tested by prior-art methods. Moreover, computer-aided design or peptidomimetics can be used for preparing suitable derivatives and analogs. The cell or the tissue which can be used for the method according to the invention is preferably a host cell, plant cell or plant tissue according to the invention as described in the abovementioned embodiments.

Derivative(s) (the plural and the singular are to be taken as equivalent for the present application and its definitions) of the nucleic acids used in the methods according to the invention are, for example, functional homologs of the proteins encoded by SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 or their biological activity, that is to say proteins which carry out the same biological reactions as the proteins encoded by SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51. These derivatives or genes are also suitable as herbicidal targets.

The sequences described herein in accordance with the invention encode homologs with the proteins described in the examples and preferably have the activities specified for the homologs.

SEQ ID NO: 1 encodes a protein with similarities to the translation realising factor RF-2. The protein sequence is shown in SEQ ID NO: 2. SEQ ID NO: 3 encodes a cobalamin synthesis protein whose protein sequence can be found in SEQ ID NO: 4. SEQ ID NO: 5 encodes an arginyl-tRNA synthetase, the protein sequence is shown in SEQ ID NO: 6. SEQ ID NO: 7 encodes a putative protein with similarity to a Mus musculus RNA helicase whose protein sequence is shown in SEQ ID NO: 8. SEQ ID NO: 9 encodes a putative protein with similarity to the Arabidopsis thaliana protein RAP 2.4, which comprises the AP2 domain and whose protein sequence can be seen from SEQ ID NO: 10. SEQ ID NO: 11 encodes a protein with homologies to various pseudouridylate synthases. The protein sequence can be seen from SEQ ID NO: 12. SEQ ID NO: 13 encodes a protein with similarities to a putative adenylate kinase. SEQ ID NO: 14 shows the protein sequence. The sequence SEQ ID NO: 15 encodes a protein with the sequence shown in SEQ ID NO: 16. This hypothetical protein encoded by SEQ ID NO: 15 has similarity to the pol polyprotein of the Equine Infectious Anemia Virus. SEQ ID NO: 17, SEQ ID NO: 19, SEQ ID NO: 21, SEQ ID NO: 35, SEQ ID NO: 43 and SEQ ID NO: 51 encode unknown proteins. The respective protein sequences can be seen from the sequences SEQ ID NO: 18, SEQ ID NO: 20, SEQ ID NO: 22, SEQ ID NO: 36, SEQ ID NO: 44 and SEQ ID NO: 52.

SEQ ID NO: 23 encodes a preprotein translocase secA precursor protein, a chloroplastidial SecA protein which is involved in the transport of proteins via the thylacoid membrane. The protein sequence can be found in SEQ ID NO: 24.

SEQ ID NO: 25 encodes a protein with significant homology to the tomato DCL protein (PIR: S71749). This protein has what is known as an HMG signature, which is found in high-mobility-group proteins and can bind to DNA. The protein sequence is represented in SEQ ID NO: 26.

SEQ ID NO: 29 encodes a plastidial glutathione reductase whose protein sequence is shown in SEQ ID NO: 30. SEQ ID NO: 31 encodes a protein which is a homolog of the transcription factor sigma, i.e. it is a plant homolog to the sigma subunit of the bacterial RNA polymerase. The corresponding protein sequence can be found in SEQ ID NO: 32.

SEQ ID NO: 33 encodes a calmodulin-like protein whose sequence is represented in SEQ ID NO: 34.

SEQ ID NO: 37 encodes a protein with great similarity to INT6, a breast-carcinoma-associated protein with similarity to an initiator factor 3 protein. SEQ ID NO: 38 represents the protein sequence.

SEQ ID NO: 39 encodes a protein with great similarity to the Saccharomyces DNA helicase YGL150c. SEQ ID NO: 40 represents the corresponding protein sequence.

SEQ ID NO: 41 encodes a protein with similarity to an RNA-binding protein. The protein sequence is represented in SEQ ID NO: 42.

SEQ ID NO: 45 encodes a putative heat shock transcription factor, whose protein sequence can be found in SEQ ID NO: 46.

SEQ ID NO: 47 encodes a putative chloroplastidial protein which binds to the DNA nucleoid. SEQ ID NO: 48 represents the corresponding protein sequence.

SEQ ID NO: 49 encodes a protein with similarity to a putative Met2-type cytosine DNA-methyltransferase. This methyltransferase has great similarities with an Arabidopsis thaliana DNA (cytosine-5-)-methyltransferase. The protein sequence is shown in SEQ ID NO: 50.

Derivatives are also understood as meaning those peptides which have at least 20%, preferably 30%, 40% or 50%, more preferably 60%, 70% or 80%, even more preferably 90%, more preferably 91%, 92%, 93%, 94% or 95%, most preferably 96%, 97%, 98% or 99% or more homology with the polypeptides with the sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which have an equivalent biological activity in other organisms and can thus be regarded as functional homologs. This functional homology or equivalence can be demonstrated for example by the possible complementation of mutants in these functions.

The abovementioned nucleic acid sequence(s) or fragments thereof can be used advantageously for isolating further sequences such as, for example, genomic, cDNA or other sequences which are suitable as herbicide target, using homology screening.

The abovementioned derivatives can be isolated for example from other organisms, in particular eukaryotic organisms such as monocotyledonous or dicotyledonous plants such as, specifically, algae, mosses, dinoflagellates, useful plants such as monocots such as maize, wheat, oats, rye, barley or sorghum/millet or dicots such as potato, tobacco, lettuce, tomato, carrot, to mention only a few, or fungi.

Derivatives or functional derivatives of the sequences stated in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 are furthermore to be understood as meaning, for example, allelic variants which have at least 60% homology, advantageously at least 70% homology, preferably at least 80% homology, especially preferably at least 85%, 90%, 91%, 92%, 93%, 94% or 95% homology, very especially preferably 96%, 97%, 98% or 99% homology at the derived amino acid level. The homology was calculated over the entire amino acid region. The programs PileUp, BESTFIT, GAP, TRANSLATE and BACKTRANSLATE (=part of the UWGCG package, Wisconsin Package, Version 10.0-UNIX, January 1999, Genetics Computer Group, Inc., Deverux et al., Nucleic. Acid Res., 12, 1984: 387-395) were used (J. Mol. Evolution., 25, 351-360, 1987, Higgins et al., CABIOS, 5 1989: 151-153). The following settings were used for nucleic acids: Gap Weight: 50, Length Weight: 3. The following settings were used for proteins: Gap Weight: 8, Length Weight: 2. The amino acid sequences derived from the abovementioned nucleic acids can be seen from SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52. Homology is to be understood as meaning identity, that is to say that the amino acid sequences have at least 40, 50, 60 or 70%, more preferably 80%, 85% or 90%, even more preferably 91%, 92%, 93%, 94% or 95%, most preferably 96%, 97%, 98% or 99% or more identity. The sequences according to the invention have at least 45 or 55% homology, preferably at least 60 or 65%, especially preferably 75% or 80%, very especially preferably at least 85% or 90%, even more preferably 95%, 96%, 97%, 98% or 99% or more homology at the nucleic acid level.

The term derivatives and the term “fragments” furthermore also encompass subregions or fragments of the abovementioned sequences or their homologous sequences of at least 50 amino acids, advantageously of at least 40 amino acids, preferably of at least 30 amino acids, especially preferably of at least 20 amino acids, very especially preferably of at least 10 amino acids, which make it possible selectively to identify interacting substances. The term fragment, “sequence fragment” or “part-sequence” denotes a truncated sequence of the original sequence. The truncated sequence (nucleic acid or protein) can have different lengths, the minimum sequence length being a sequence length which has at least one comparable function, for example binding properties, or activity of the original sequence. Such methods are, for example, SELDI, FCS or Biocore as described above, which are known to the skilled worker.

Equally encompassed are thus nucleic acids which encode a fragment or an epitope of a polypeptide which specifically binds to an antibody which specifically binds to a polypeptide described in accordance with the invention, in particular which is encoded by one of the sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51. Fragments or epitopes of a polypeptide which specifically interact with such an antibody have a significant homology with regard to the spatial structure to the polypeptides described herein, at least in subregions. Preferably, they also have high homology at the amino acid level with the abovementioned sequences, preferably 20%, with 40% being more preferred, 60% more preferred, 80% even more preferred, but 90% or more being most preferred. The spatial structure of a polypeptide, however, is essentially one of the factors responsible for the interactions of the polypeptide with other compounds and, if appropriate, for its enzymatic activity. Accordingly, in the processes according to the invention fragments may be employed whose sequence has only a low degree of homology with the above-described polypeptides, but whose spatial structure has a high degree of homology with the above-described polypeptides, that is to say those comprising epitopes of the sequences described herein, in order to find interactants which then inhibit or inactivate the polypeptides described herein. Fragments which encompass epitopes of the polypeptides according to the invention can also be used to “occupy” the interactants of the polypeptides according to the invention, i.e. to prevent their interaction with the polypeptides according to the invention. To this end, it is advantageous for the fragments to have a greater affinity to a binding partner than the naturally occurring polypeptide. Likewise encompasssed are fragments which are encoded by nucleic acids according to the invention and which encompass one of the abovementioned biological activities.

Allelic variants encompass in particular functional variants which can be obtained from the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 by deletion, insertion or substitution of nucleotides, the biological, e.g. enzymatic activity or binding properties of the derived proteins which are synthesized being retained.

Starting from, for example, the DNA sequences described in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 or parts of these sequences, such DNA sequences can be isolated from other eukaryotic organisms such as, for example, microorganisms such as yeasts, fungi, ciliates, plants such as algae, mosses or other plants, with the aid of the nucleic acid sequences according to the invention, for example using customary hybridization methods or PCR technology. These DNA sequences hybridize with the abovementioned sequences under standard conditions. For hybridization, it is advantageous to use short oligonucleotides, for example of the conserved or other regions, which can be determined via alignment with other related genes in the manner known to the skilled worker. However, longer fragments of the nucleic acids according to the invention or the complete sequences may also be used for hybridization. These standard conditions vary depending on the nucleic acid used: oligonucleotide, longer fragment or complete sequence, or on the type of nucleic acid, DNA or RNA, which is used for the hybridization. Thus, for example, the melting points for DNA: DNA hybrids are approximately 10° C. lower than those of DNA:RNA hybrids of the same length.

Standard conditions are to be understood as meaning, for example, temperatures between 42 and 58° C. in an aqueous buffer solution with a concentration of between 0.1 to 5×SSC (1×SSC=0.15 M NaCl, 15 mM sodium citrate, pH 7.2) or additionally in the presence of 50% formamide such as, for example, 42° C. in 5×SSC, 50% formamide, depending on the nucleic acid. The hybridization conditions for DNA:DNA hybrids are advantageously 0.1×SSC and temperatures of between approximately 20° C. and 45° C., preferably between approximately 30° C. and 45° C. For DNA:RNA hybrids, the hybridization conditions are advantageously 0.1×SSC and temperatures of between approximately 30° C. and 55° C., preferably between approximately 45° C. and 55° C. These temperatures stated for the hybridization are examples of calculated melting point values for a nucleic acid with a length of approximately 100 nucleotides and a G+C content of 50% in the absence of formamide. The experimental conditions for DNA hybridization are described in specialist textbooks of genetics such as, for example, Sambrook et al., “Molecular Cloning”, Cold Spring Harbor Laboratory, 1989, and can be calculated by formulae known to the skilled worker, for example as a function of the length of the nucleic acids, the type of the hybrids or the G+C content. The skilled worker will find further information on hybridization in the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

Derivatives are furthermore to be understood as meaning homologs of the sequence SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51, for example eukaryotic homologs, truncated sequences, simplex DNA of the coding and noncoding DNA sequence or RNA of the coding and noncoding DNA sequence.

Homologs of the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 are furthermore understood as meaning derivatives such as, for example, variants from other organisms, for example other plants. These variants can be modified by one or more nucleotide substitutions, by insertion(s) and/or deletion(s) without, however, adversely affecting the functionality or biological activity of the variants. They preferably have a homology of at least 20%, advantageously 30%, 40%, 50% or 60%, preferably 70%, 80% or 90%, particularly preferably 95% and an equivalent biological activity.

The nucleic acids which are used in the method according to the invention, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 and their fragments and derivatives are therefore advantageously suitable for isolating further essential, novel genes from other organisms, preferably plants.

The nucleic acid sequences according to the invention, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 and the gene products which are encoded by them are used in the method according to the invention. They can be of synthetic or natural origin or comprise a mixture of synthetic and natural DNA components, or else be composed of various heterologous gene segments of different organisms. In general, synthetic nucleotide sequences are prepared which have codons which are preferred by the host organisms in question, for example plants. As a rule, this leads to optimal expression of the heterologous genes. These codons which are preferred by plants can be determined from codons with the highest protein frequency which are expressed in most of the plant species of interest. An example of Corynebacterium glutamicum is provided in: Wada et al. (1992) Nucleic Acids Res. 20:2111-2118). Such experiments can be carried out with the aid of standard methods and are known to those skilled in the art.

Functionally equivalent sequences which encode the nucleic acids used in the method according to the invention are those derivatives of the sequences according to the invention which, despite deviating nucleotide sequence, retain the desired functions, that is to say the biological activity of the proteins. Functional equivalents thus encompass naturally occurring variants of the sequences described herein, and also artificial nucleotide sequences, for example artificial nucleotide sequences which have been obtained by chemical synthesis and which are, in particular, adapted to the codon usage of a plant.

Furthermore suitable are artificial DNA sequences as long as, as described above, they lead to products which mediate the abovementioned activities or the desired property, for example binding to a receptor or enzymatic activity. Such artificial DNA sequences can be determined, for example, by backtranslating proteins which have been constructed by means of molecular modeling, or by in vitro selection. Possible techniques for the in-vitro evolution of DNA for modifying or improving the DNA sequences are described by Patten, P. A. et al., Current Opinion in Biotechnology 8, 724-733(1997) or by Moore, J. C. et al., Journal of Molecular Biology 272, 336-347(1997). Especially suitable are coding DNA sequences which are obtained by backtranslating a polypeptide sequence in accordance with the codon usage which is specific for the host plant. The specific codon usage can be determined readily by a skilled worker who is familiar with plant genetic methods by means of computer evaluations of other, known genes of the plant to be transformed.

Amino acid sequences which are to be understood as advantageous for the method according to the invention are those comprising an amino acid sequence shown in sequences SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 or a sequence which can be obtained from these by substitution, inversion, insertion or deletion of one or more amino acid residues, the biological activity of the protein shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 being retained or not being reduced substantially. The term not substantially reduced refers to all those proteins which retain at least 10%, preferably 20%, especially preferably 30%, 50%, 70%, 90% or more of the biological activity of the original protein. In this context, particular amino acids can, for example, be replaced by those with similar physicochemical properties (spatial arrangement, basicity, hydrophobicity and the like). For example, arginine residues are exchanged for lysine residues, valine residues for isoleucine residues or aspartate residues for glutamate residues. However, a sequence of one or more amino acids may also be swapped, one or more amino acids may be added or removed, or several of these measures can be combined with each other.

Derivatives are also to be understood as meaning functional equivalents which encompass in particular also natural or artificial mutations of the nucleic acid sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 used, which furthermore retain the desired function, that is to say that their biological activity is not substantially reduced. Mutations encompass substitutions, additions, deletions, exchanges or insertions of one or more nucleotide residues. Thus, the present invention encompasses, for example, also those nucleotide sequences which are obtained by modifying the abovementioned nucleotide sequences. The aim of such a modification can be, for example, the further delimitation of the coding sequence comprised therein or else, for example, the insertion of further cleavage sites for restriction enzymes.

Functional equivalents are also those variants whose function, compared with the original gene or gene fragment, is weakened (=not substantially reduced) or increased (=enzyme activity greater than the activity of the original enzyme, that is to say the activity is higher than 100%, preferably higher than 150%, especially preferably higher than 180%).

In this context, the nucleic acid sequence can advantageously be, for example, a DNA or cDNA sequence. Coding sequences which are suitable for insertion into a nucleic acid construct according to the invention (=expression cassette or nucleic acid fragment) are, for example, those which encode a protein with the above-described sequences and which impart, to the host, the ability to overproduce the protein and thus its biological function. These sequences can be of homologous or heterologous origin.

The invention therefore furthermore relates to a nucleic acid construct containing a nucleic acid sequence according to the invention selected, for example, from the group consisting of:
a) a nucleic acid sequence with the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51;
b) a nucleic acid sequence which can be derived from the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 by backtranslation owing to the degeneracy of the genetic code;
c) a nucleic acid sequence which is a derivative or a fragment of the nucleic acid sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 and which have at least 60% homology at the nucleic acid level; or
d) a nucleic acid sequence which encodes derivatives or fragments of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which have at least 50% homology at the amino acid level;
e) a nucleic acid sequence which encodes a fragment or an epitope of a polypeptide which binds specifically to an antibody, the antibody specifically binding to a polypeptide which is encoded by the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51;
f) a nucleic acid sequence which encodes a fragment of a nucleic acid shown in a) and which has a translation releasing factor activity, a cobalamin synthase activity, an arginyl-tRNA synthase activity, an RNA helicase activity, a GTP binding protein activity, a pseudouridylate synthase activity, an adenylate kinase activity, a preprotein translocase secA precursor protein activity, a DCL protein activity, an arginine-tRNA ligase activity, a plastidial glutathione reductase activity, a transcription factor sigma activity, a calmodulin activity, an INT6 activity, a helicase YGL150c activity, an RNA-binding activity, a heat shock transcription factor activity, a chloroplastidial DNA nucleoid binding activity or a Met2-type cytosine DNA methyltransferase activity; and/or
g) a nucleic acid sequence which encodes derivatives of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which has at least 20% homology at the amino acid level and has an equivalent biological activity;
   the nucleic acid sequence being linked to one or more regulatory signals. The abovementioned terms have the abovementioned meanings.

The nucleic acid construct according to the invention is to be understood as meaning the nucleic acids according to the invention, e.g., the sequences stated in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 which as the result of the genetic code and/or their functional or nonfunctional derivatives which were functionally linked to one or more regulatory signals advantageously for regulating, in particular for increasing gene expression and which govern the expression of the coding sequence in the host cell. These regulatory sequences are intended to make possible the targeted expression of the genes, or proteins. Depending on the host organism, this may mean, for example, that the gene is expressed and/or overexpressed only after induction, or that it is expressed and/or overexpressed constitutively. For example, these regulatory sequences take the form of sequences to which inductors or repressors bind, thus regulating the expression of the nucleic acid. In addition to these novel regulatory sequences, or instead of these sequences, the natural regulation of these sequences may still be present before the actual structural genes and, if appropriate, have been modified genetically so that the natural regulation has been switched off and the expression of the genes increased. The nucleic acid construct according to the invention may also advantageously only be composed of the natural recombinantly modified regulatory region at the 5′ and/or 3′ end. However, the gene construct may also be constructed in a simpler fashion, that is to say no additional regulatory signals were inserted before the nucleic acid sequence or its derivatives and the natural promoter with its regulation was not removed. Instead, the natural regulatory sequence was mutated so that regulation no longer takes place and/or gene expression is increased. To increase the activity, these modified promoters may also be introduced before the natural gene by themselves in the form of part-sequences (=promoter with portions of the nucleic acid sequences according to the invention). Moreover, the gene construct can advantageously also comprise one or more of what are known as “enhancer sequences” functionally linked to the promoter, and these make possible an increased expression of the nucleic acid sequence. Additional advantageous sequences such as further regulatory elements or terminators may also be inserted at the 3′ end of the DNA sequences. The nucleic acid sequences used in the method according to the invention may be present in the expression cassette (=gene construct) in one or more copies.

As described above, the regulatory sequences or factors can preferably exert a positive effect on, and thus increase, the gene expression of the genes which have been introduced. Thus, an enhancement of the regulatory elements may advantageously take place at the transcription level, by using strong transcription signals such as promoters and/or enhancers. In addition, however, increased translation is also possible, for example by improving the stability of the mRNA. In another advantageous embodiment, however, expression may also be reduced or blocked in a targeted fashion.

Promoters which are suitable as promoters in the expression cassette are, in principle, all those which are capable of governing the expression of foreign genes in organisms, advantageously in plants or fungi. In particular plant promoters or promoters originating from a plant virus are used by preference. Advantageous regulatory sequences for the method according to the invention are present, for example, in promoters such as the cos, tac, trp, tet, trp-tet, lpp, lac, lpp-lac, lacl^{q}, T7, T5, T3, gal, trc, ara, SP6, λ-P_{R} or in the λ-P_{L} promoter, these promoters being used advantageously in Gram-negative bacteria. Further advantageous regulatory sequences are present, for example, in the Gram-positive promoters amy and SPO2, in the yeast or fungal promoters ADC1, MFα, AC, P-60, CYC1, GAPDH, TEF, rp28, ADH or in the plant promoters such as in the CaMV/35S [Franck et al., Cell 21(1980) 285-294], SSU, OCS, lib4, STLS1, B33, nos (=nopaline synthase promoter) or in the ubiquitin promoter. The expression cassette may also comprise a chemically inducible promoter by which the expression of the nucleic acid sequences in the nucleic acid construct according to the invention can be controlled in the organisms, advantageously in the plants, at a particular point in time. Such advantageous plant promoters are, for example, the PRP1 promoter [Ward et al., Plant. Mol. Biol. 22(1993), 361-366], a benzenesulfonamide-inducible promoter (EP 388186), a tetracycline-inducible promoter (Gatz et al., (1992) Plant J. 2, 397-404), a salicylic-acid-inducible promoter (WO 95/19443), an abscisic-acid-inducible promoter (EP 335528) or an ethanol- or cyclohexanone-inducible promoter (WO93/21334). Further plant promoters are, for example, the potato cytosolic FBPase promoter, the potato ST-LSI promoter (Stockhaus et al., EMBO J. 8 (1989) 2445-245), the Glycine max phosphoribosyl-pyrophosphate amidotransferase promoter (see also Genbank Accession Number U87999) or a node-specific promoter such as in EP 249676 can advantageously be used.

As described above, further genes to be introduced into the organism may also be present in the expression cassette (=gene construct, nucleic acid construct). These genes can be subject to separate regulation or subject to the same regulatory region as the nucleic acid sequences used in the method. For example, these genes take the form of biosynthesis genes of the metabolism, such as genes which participate in the metabolic pathways of the proteins encoded by the nucleic acids according to the invention. However, they may also be biosynthesis genes of other metabolic pathways such as of fatty acid, amino acid or vitamin biosynthesis, or regulatory genes, to mention just a few.

In principle, all natural promoters together with their regulatory sequences, such as those mentioned above, can be used for the expression cassette according to the invention and for the method according to the invention, as described hereinbelow. Moreover, synthetic promoters may also be used advantageously.

When preparing an expression cassette, various DNA fragments can be manipulated in order to obtain a nucleotide sequence which expediently reads in the correct direction and is equipped with a correct reading frame. To connect the DNA fragments (=nucleic acids according to the invention) to each other, adapters or linkers may be attached to the fragments.

The promoter and terminator regions can expediently be provided, in the direction of transcription, with a linker or polylinker containing one or more restriction sites for the insertion of this sequence. As a rule, the linker has 1 to 10, in most cases 1 to 8, preferably 2 to 6, restriction sites. In general, the linker within the regulatory regions has a size of less than 100 bp, frequently less than 60 bp, but at least 5 bp. The promoter can be both native, or homologous, and foreign, or heterologous, with regard to the host organism, for example the host plant. In the 5′-3′ direction of transcription, the expression cassette comprises the promoter, a DNA sequence which encodes the proteins used in the method according to the invention, and a region for transcriptional termination. Various termination regions can advantageously be exchanged for each other.

Furthermore, manipulations which provide suitable restriction cleavage sites or which remove surplus DNA or restriction cleavage sites may be employed. Where insertions, deletions or substitutions such as, for example, transitions and transversions are suitable, in vitro mutagenesis, primer repair, restriction or ligation may be used. In the case of suitable manipulations such as, for example, restriction, chewing back or filling in overhangs for blunt ends, complementary ends of the fragments may be provided for ligation.

Attaching the specific ER retention signal SEKDEL (Schouten, A. et al., Plant Mol. Biol. 30 (1996), 781-792) may, inter alia, be of importance for an advantageous high level of expression; the average expression level is tripled to quadrupled thereby. Other retention signals which occur naturally in vegetable and animal proteins located in the ER may also be employed for synthesizing the cassette.

Preferred polyadenylation signals are plant polyadenylation signals, preferably those which essentially correspond to T-DNA polyadenylation signals from Agrobacterium tumefaciens, in particular of gene 3 of the T-DNA (octopine synthase) of the Ti plasmid pTiACH5 (Gielen et al., EMBO J. 3 (1984), 835 et seq.) or suitable functional equivalents.

An expression cassette is generated by fusing a suitable promoter to a suitable nucleic acid sequence and a polyadenylation signal, using customary recombination and cloning techniques as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

When preparing an expression cassette, various DNA fragments may be manipulated in order to obtain a nucleotide sequence which expediently reads in the correct direction and which is equipped with a correct reading frame. To link the DNA fragments to each other, adapters or linkers may be attached to the fragments.

The nucleic acid sequences used in the method according to the invention encompass all sequence characteristics which are necessary to achieve a localization which is correct for the site of the biological action or activity. Thus, further targeting sequences are not necessary per se. However, such a localization may be desirable and advantageous and may therefore be modified or enhanced artificially so that such fusion constructs are also a preferred advantageous embodiment of the invention.

Advantageous for this purpose are, for example, sequences which ensure targeting into plastids. Under certain circumstances, targeting into other compartments (reviewed in: Kermode, Crit. Rev. Plant Sci. 15, 4 (1996), 285-423), for example into the vacuole, into the mitochondrion, into the endoplasmic reticulum (ER), peroxisomes, lipid bodies or else, owing to the absence of suitable operative sequences, remaining in the compartment of formation, the cytosol, may also be desirable.

Advantageously, the nucleic acid sequences according to the invention, together with at least one reporter gene, are cloned into an expression cassette which is introduced into the organism via a vector or directly into the genome. This reporter gene should allow easy detectability via a growth, fluorescence, chemoluminescence, bioluminescence or resistance assay or via a photometric measurement. Examples of reporter genes which may be mentioned are genes for resistance to antibiotics or herbicides, hydrolase genes, fluorescence protein genes, bioluminescence genes, sugar or nucleotide metabolism genes, or biosynthesis genes such as the Ura3 gene, the Ilv2 gene, the luciferase gene, the β-galactosidase gene, the gfp gene, the 2-deoxyglucose-6-phosphate phosphatase gene, the β-glucuronidase gene, the β-lactamase gene, the neomycin phosphotransferase gene, the hygromycin phosphotransferase gene, or the gene for BASTA (=glufosinate resistance). Further advantageous antibiotic or herbicidal resistances are resistance to, for example, imidazolinone or sulfonylurea; the antibiotic resistances to, for example, bleomycin, streptomycin, kanamycin, tetracyclin, chloramphenicol, gentamycin, geneticin (G418), spectinomycin or blasticidin, to mention just a few. These genes allow the transcription activity, and thus gene expression, to be measured and quantified readily. This makes possible the identification of sites in the genome which show different productivity.

In a preferred embodiment, an expression cassette comprises upstream, i.e. at the 5′ end of the coding sequence, a promoter and downstream, i.e. at the 3′ end, a polyadenylation signal and, if appropriate, further regulatory elements which are linked operably to the interposed coding sequence for the proteins used in the method according to the invention. Operable linkage is to be understood as meaning the sequential arrangement of the promoter, coding sequence, terminator and, if appropriate, further regulatory elements in such a way that each of the regulatory elements can fulfill its intended function upon expression of the coding sequence. The sequences which are preferred for operable linkage are targeting sequences for ensuring subcellular localization in plastids. However, targeting sequences for ensuring subcellular localization in the mitochondrion, in the endoplasmic reticulum (=ER), in the nucleus, in elaioplasts or other compartments may also be used, if required, as may translation enhancers such as the tobacco mosaic virus 5′ leader sequence (Gallie et al., Nucl. Acids Res. 15 (1987), 8693-8711).

An expression cassette may, for example, comprise a constitutive promoter, for example the 35S, 34S or a ubiquitin promoter, the gene to be expressed, and the ER retention signal. The amino acid sequence KDEL (lysine, aspartic acid, glutamic acid, leucine) is preferably used as ER retention signal.

For expression in a prokaryotic or eukaryotic host organism, for example a microorganism such as a fungus, or a plant, the expression cassette is advantageously inserted into a vector such as, for example, a plasmid, a phage or other DNA which makes possible optimal expression of the genes in the host organism. Suitable plasmids are, for example, in E. coli pLG338, pACYC184, pBR series, such as, for example, pBR322, pUC series, such as pUC18 or pUC19, M113 mp series, pKC30, pRep4, pHS1, pHS2, pPLc236, pMBL24, pLG200, pUR290, pIN-III¹¹³-B1, λgt11 or pBdCl, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, further advantageous fungal vectors are described by Romanos, M. A. et al., [(1992) “Foreign gene expression in yeast: a review”, Yeast 8: 423-488] and by van den Hondel, C. A. M. J. J. et al. [(1991) “Heterologous gene expression in filamentous fungi”] and in More Gene Manipulations in Fungi [J. W. Bennet & L. L. Lasure, eds., p. 396-428: Academic Press: San Diego] and in “Gene transfer systems and vector development for filamentous fungi” [van den Hondel, C. A. M. J. J. & Punt, P. J. (1991) in: Applied Molecular Genetics of Fungi, Peberdy, J. F. et al., eds., p. 1-28, Cambridge University Press: Cambridge]. Advantageous yeast promoters are, for example, 2 μM, pAG-1, YEp6, YEp13 or pEMBLYe23. Examples of algal or plant promoters are pLGV23, pGHlac⁺, pBIN19, pAK2004, pVKH or pDH51 (see Schmidt, R. and Willmitzer, L., 1988). The abovementioned vectors or derivatives of the abovementioned vectors constitute a small selection of the plasmids which are possible. Further plasmids are well known to the skilled worker and can be found, for example, in the book Cloning Vectors (Eds. Pouwels P. H. et al. Elsevier, Amsterdam-New York-Oxford, 1985, ISBN 0 444 904018). Suitable plant vectors are described, inter alia, in “Methods in Plant Molecular Biology and Biotechnology” (CRC Press), chapter 6/7, pp. 71-119. Advantageous vectors are what are known as shuttle vectors or binary vectors, which replicate in E. coli and Agrobacterium.

In addition to plasmids, vectors are also to be understood as meaning all of the other vectors known to the skilled worker, such as, for example, phages, viruses such as SV40, CMV, baculovirus, adenovirus, transposons, IS elements, phasmids, phagemids, cosmids, linear or circular DNA. These vectors can be replicated autonomously in the host organism or can be replicated chromosomally; chromosomal replication is preferred. Functional and nonfunctional vectors are encompassed.

In a further embodiment of the vector, the nucleic acid construct according to the invention may also advantageously be introduced into the organisms in the form of a linear DNA and integrated into the genome of the host organism via heterologous or homologous recombination. This linear DNA may be composed of a linearized plasmid or only of the nucleic acid construct as vector, or the nucleic acid sequences used.

In a further advantageous embodiment, the nucleic acid sequences used in the method according to the invention may also be introduced into an organism by themselves.

If, in addition to the nucleic acid sequences, further genes are to be introduced into the organism, all may be introduced into the organism together with a reporter gene in a single vector, or each individual gene with or without a reporter gene in a separate vector, it being possible to introduce the various vectors simultaneously or in succession.

The vector advantageously comprises at least one copy of the nucleic acid sequences used and/or of the nucleic acid construct according to the invention.

For example, the nucleic acid construct can be incorporated into the tobacco transformation vector pBinAR and be under the control of the 35S, 34S or ubiquitin promoter or the USP promoter.

As an alternative, a recombinant vector (=expression vector) may also be transcribed and translated in vitro, for example by using the T7 promoter and T7 RNA polymerase.

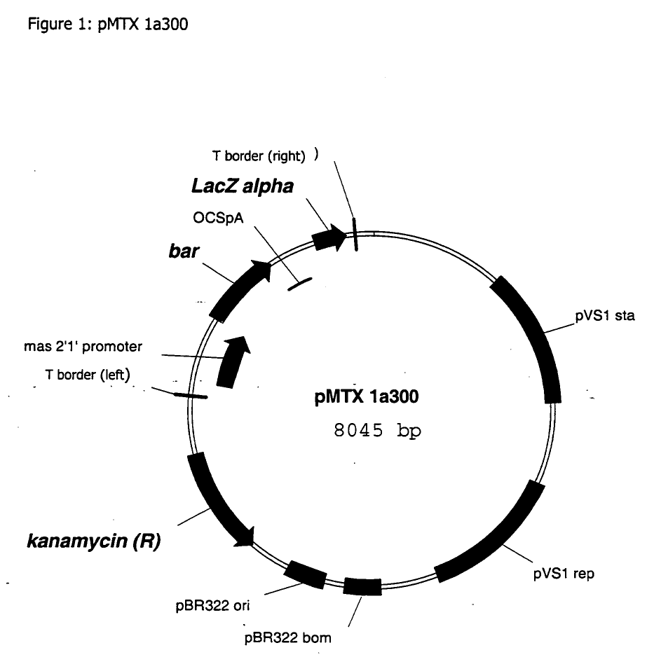

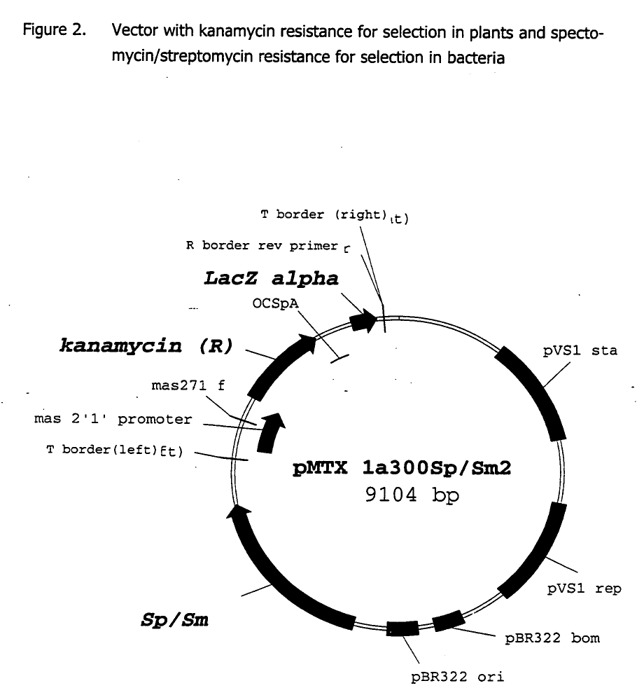

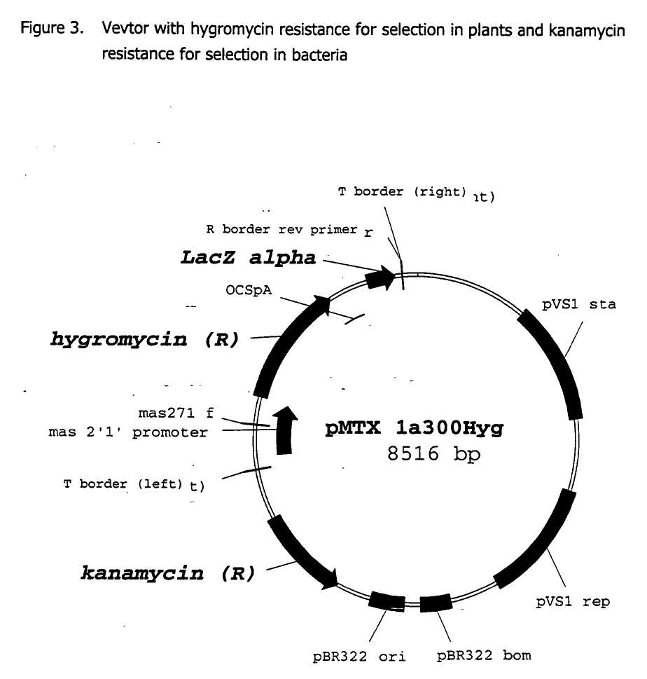

Further advantageous vectors comprise resistances which can be used in plants or plant crops, such as the resistance to phosphinothricin (=bar resistance), the resistance to methionine sulfoximine, the resistance to sulfonylurea (=ilv resistance, ind S. cerevisiae ilv2), the resistance to phenoxyphenoxy herbicide (=ACCase resistance), the resistance to glyphosate or Clearfield (AHAS resistance), or the genes which encode these resistances. These resistances can be exploited in intact plants for selecting transgenic plants. Only plants to which these resistances have been imparted via a transformation process are capable of growing in the presence of the selecting substance. Following transformation in planta—for example infiltration of the seed precursor cells—kanamycin or hygromycin are other examples of selecting agents in cell cultures on agar plates. Moreover, advantageous vectors may comprise sequences for integration into the genome of the organisms, preferably the plants. Examples of such sequences are what are known as T-DNA borders. In addition, advantageous vectors may also comprise promoters and terminators such as, for example, those described above. What are known as poly-A sequences may also be present in the vector. Advantageous vectors can be found, for example, in FIGS. 1, 2 and 3. SEQ ID NO: 25 indicates the advantageous sequence of vector pMTX 1a300. This vector contains a kanamycin resistance (nucleotide 4922-5713), a phosphinothricin resistance (nucleotide 6722-7288), the LacZalpha fragment (nucleotide 7630-7864), a portion of pVS1sta (nucleotide 945-1945), a portion of pBR322bom (nucleotide 39484208), a T border sequence (left, nucleotide 6138-6163), a T border sequence (right, nucleotide 7924-7949), a poly-A portion (nucleotide 7292-7503), the mas2′1′ promoter (nucleotide 6241-6718) and two origins of replication pVS1 rep (nucleotide 6241-6718) and pBR322ori (nucleotide 43-4628).

Expression vectors used in prokaryotes frequently exploit inducible systems with and without fusion proteins or fusion oligopeptides, it being possible for these fusions to be effected at the N terminal or the C terminal or other utilizable domains of a protein. In general, the purpose of such fusion vectors is: i.) to increase the expression rate of the RNA, ii.) to increase the achievable protein synthesis rate, iii.) to increase the solubility of the protein, or iv.) to simplify purification by a binding sequence which can be exploited in affinity chromatography. Also, proteolytic cleavage sites are frequently introduced via fusion proteins, which makes possible the elimination of a portion of the fusion protein after purification. Such recognition sequences which proteases recognize are, for example, factor Xa, thrombin and enterokinase.

Typical advantageous fusion and expression vectors are pGEX [Pharmacia Biotech Inc; Smith, D. B. and Johnson, K. S. (1988) Gene 67:31-40], pMAL (New England Biolabs, Beverly, Mass.) and pRIT5 (Pharmacia, Piscataway, N.J.), which comprises glutathione S transferase (GST), maltose binding protein, or protein A.

Further examples for E. coli expression vectors are pTrc [Amann et al., (1988) Gene 69:301-315] and pET vectors [Studier et al., Gene Expression Technology: Methods in Enzymology 185, Academic Press, San Diego, Calif. (1990) 60-89; Stratagene, Amsterdam, Netherlands].

Further advantageous vectors for use in yeast are pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), and pYES derivatives (Invitrogen Corporation, San Diego, Calif.). Vectors for use in filamentous fungi are described in: van den Hondel, C. A. M. J. J. & Punt, P. J. (1991) “Gene transfer systems and vector development for filamentous fungi”, in: Applied Molecular Genetics of Fungi, J. F. Peberdy, et al., eds., p. 1-28, Cambridge University Press: Cambridge.

As an alternative, insect cell expression vectors may also be used advantageously, for example for expression in Sf 9 cells. Examples of these are the vectors of the pAc series (Smith et al. (1983) Mol. Cell Biol. 3:2156-2165) and of the pVL series (Lucklow and Summers (1989) Virology 170:31-39).

Moreover, plant cells or algal cells may advantageously be used for gene expression. Examples of plant expression vectors are found in Becker, D., et al. (1992) “New plant binary vectors with selectable markers located proximal to the left border”, Plant Mol. Biol. 20: 1195-1197 or in Bevan, M. W. (1984) “Binary Agrobacterium vectors for plant transformation”, Nucl. Acid. Res. 12: 8711-8721.

Furthermore, the nucleic acid sequences according to the invention can be expressed in mammalian cells. Examples of suitable expression vectors are pCDM8 and pMT2PC, which are mentioned in: Seed, B. (1987) Nature 329:840 or Kaufman et al. (1987) EMBO J. 6:187-195). Promoters preferably to be used are of viral origin, such as, for example, promoters of polyoma virus, adenovirus 2, cytomegalovirus or simian virus 40. Further prokaryotic and eukaryotic expression systems are mentioned in chapters 16 and 17 in Sambrook et al., Molecular Cloning: A Laboratory Manual. 2nd, ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

In principle, the nucleic acids according to the invention, the expression cassette or the vector can be introduced into organisms, for example into plants, by all methods with which the skilled worker is familiar.

For microorganisms, the skilled worker will find suitable methods in the textbooks by Sambrook, J. et al. (1989) Molecular cloning: A laboratory manual, Cold Spring Harbor Laboratory Press, by F. M. Ausubel et al. (1994) Current protocols in molecular biology, John Wiley and Sons, by D. M. Glover et al., DNA Cloning Vol. 1, (1995), IRL Press (ISBN 019-963476-9), by Kaiser et al. (1994) Methods in Yeast Genetics, Cold Spring Habor Laboratory Press or Guthrie et al. Guide to Yeast Genetics and Molecular Biology, Methods in Enzymology, 1994, Academic Press.

The transfer of foreign genes into the genome of a plant is referred to as transformation. It exploits the above-described methods of transforming and regenerating plants from plant tissues or plant cells for transient or stable transformation. Suitable methods are protoplast transformation by polyethylene glycol-induced DNA uptake, the biolistic method with the gene gun-known as the particle bombardment method-, electroporation, incubation of dry embryos in DNA-containing solution, microinjection and Agrobacterium-mediated gene transfer. In the present invention, the gene transfer is advantageously effected using, for example, Agrobacterium tumefaciens strain GV 3101 pMP90. The abovementioned methods are described in, for example, B. Jenes et al., Techniques for Gene Transfer, in: Transgenic Plants, Vol. 1, Engineering and Utilization, edited by S. D. Kung and R. Wu, Academic Press (1993) 128-143 and in Potrykus Annu. Rev. Plant Physiol. Plant Molec. Biol. 42 (1991) 205-225. The construct to be expressed is preferably cloned into a vector which is suitable for transforming Agrobacterium tumefaciens, for example pBin19 (Bevan et al., Nucl. Acids Res. 12 (1984) 8711). Agrobacteria transformed with such a vector can then be used for transforming plants, in particular crop plants such as, for example, tobacco plants, in the known manner, for example by bathing scarified leaves or leaf sections in an agrobacterial solution and subsequently growing them in suitable media. The transformation of plants with Agrobacterium tumefaciens is described, for example, by Höfgen and Willmitzer in Nucl. Acid Res. (1988) 16, 9877 or is known, inter alia, from F. F. White, Vectors for Gene Transfer in Higher Plants; in Transgenic Plants, Vol. 1, Engineering and Utilization, edited by S. D. Kung and R. Wu, Academic Press, 1993, pp. 15-38.

An advantageous embodiment is described hereinbelow. If agrobacteria are used for the transformation, the nucleic acid or DNA to be introduced will be cloned into specific plasmids, either into an intermediary vector or into a binary vector. The intermediary vectors can be integrated into the Ti or Ri plasmid of the agrobacteria by homologous recombination, owing to sequences which are homologous to sequences in the T-DNA. The Ti or Ri plasmid additionally comprises the vir region, which is required for the transfer of the T-DNA. Intermediary vectors are not capable of replication in agrobacteria. The intermediary vector can be transferred to Agrobacterium tumefaciens by means of a helper plasmid (conjugation). Binary vectors are capable of replication both in E. coli and in agrobacteria. They comprise a selection marker gene and a linker or polylinker, which are framed by the right and left T-DNA border region. They can be transformed directly into the agrobacteria (Holsters et al. Mol. Gen. Genet. 163 (1978), 181-187). The agrobacterium which acts as the host cell should comprise a plasmid carrying a vir region. The vir region is required for the transfer of the T-DNA into the plant cell. Additional T-DNA may be present. The agrobacterium transformed in this way is used for transforming plant cells.

The use of T-DNA for transforming plant cells has been studied intensively and described amply in EPA-0 120 516; Hoekema, In: The Binary Plant Vector System Offsetdrukkerij Kanters B. V., Alblasserdam (1985), Chapter V; Fraley et al., Crit. Rev. Plant. Sci., 4: 146 and An et al. EMBO J. 4 (1985), 277-287.

To transfer the DNA into the plant cell, plant explants can expediently be cocultured with Agrobacterium tumefaciens or Agrobacterium rhizogenes. Then, intact plants can be regenerated from the infected plant material (for example leaf sections, stem segments, roots, but also protoplasts, or plant cells grown in suspension culture) in a suitable medium which may comprise antibiotics or biocides for selecting transformed cells. The plants obtained in this way can then be examined for the presence of the DNA introduced. Other possibilities of introducing foreign DNA using the biolistic method or by protoplast transformation are known (cf., for example, Willmitzer, L., 1993 Transgenic plants. In: Biotechnology, A Multi-Volume Comprehensive Treatise (H. J. Rehm, G. Reed, A. Pühler, P. Stadler, eds.), Vol. 2, 627-659, VCH Weinheim-New York-Basel-Cambridge).

The transformation of monocotyledonous plants by means of Agrobacterium-based vectors has also been described (Chan et al, Plant Mol. Biol. 22(1993), 491-506; Hiei et al, Plant J. 6 (1994) 271-282; Deng et al.; Science in China 33 (1990), 28-34; Wilmink et al., Plant Cell Reports 11, (1992) 76-80; May et al.; Biotechnology 13 (1995) 486-492; Conner and Domisse; Int. J. Plant Sci. 153 (1992) 550-555; Ritchie et al.; Transgenic Res. (1993) 252-265). Alternative systems for transforming monocotyledonous plants are the transformation by means of the biolistic approach (Wan and Lemaux; Plant Physiol. 104 (1994), 37-48; Vasil et al.; Biotechnology 11 (1992), 667-674; Ritala et al., Plant Mol. Biol. 24, (1994) 317-325; Spencer et al., Theor. Appl. Genet. 79 (1990), 625-631), protoplast transformation, the electroporation of partially permeabilized cells, the introduction of DNA by means of glass fibers. In particular the transformation of maize has been described repeatedly in the literature (cf., for example, WO 95/06128; EP 0513849 A1; EP 0465875 A1; EP 0292435 A1; Fromm et al., Biotechnology 8 (1990), 833-844; Gordon-Kamm et al., Plant Cell 2 (1990), 603-618; Koziel et al., Biotechnology 11 (1993) 194-200; Moroc et al., Theor Applied Genetics 80 (190) 721-726).

The successful transformation of other cereal species has also been described, for example in the case of barley (Wan and Lemaux, see above; Ritala et al., see above; wheat (Nehra et al., Plant J. 5(1994) 285-297).

Agrobacteria transformed with a vector according to the invention can also be used in the known manner for transforming plants such as test plants such as Arabidopsis or crop plants such as cereals, maize, oats, rye, barley, wheat, soybean, rice, cotton, sugar beet, canola, sunflower, flax, hemp, potato, tobacco, tomato, carrot, capsicum, oilseed rape, tapioca, cassaya, arrowroot, Tagetes, alfalfa, lettuce and the various tree, nut and grapevine species, for example by bathing scarified leaves or leaf segments in an agrobacterial solution and subsequently growing them in suitable media.

The genetically modified plant cells can be regenerated via all methods known to the skilled worker. Suitable methods can be found in the abovementioned publications by S. D. Kung and R. Wu, Potrykus or Höfgen and Willmitzer.

For the purposes of the invention, plants are to be understood as meaning plant cells, plant tissue, plant organs or intact plants such as seeds, tubers, flowers, pollen, fruits, seedlings, roots, leaves, stems or other plant parts. Moreover, plants are to be understood as meaning propagation material such as seeds, fruits, seedlings, slips, tubers, cuttings or rootstocks.

In principle, suitable organisms or host organisms for the nucleic acid according to the invention, the expression cassette or the vector are advantageously all organisms which are capable of expressing the nucleic acids used in accordance with the invention or which are suitable for the expression of recombinant genes. Plants which may be mentioned by way of example are Arabidopsis, Asteraceae such as Calendula, or crop plants such as soybean, peanut, castor-oil plant, sunflower, maize, cotton, flax, oilseed rape, coconut, oil palm, safflower (Carthamus tinctorius) or cocoa bean, microorganisms such as fungi, for example the genus Mortierella, Saprolegnia or Pythium, bacteria such as the genus Escherichia, yeasts such as the genus Saccharomyces, cyanobacteria, ciliates, algae or protozoans such as dinoflagellates, such as Crypthecodinium. Organisms which naturally synthesize substantial amounts of oils and which may be mentioned by way of example are soybean, oilseed rape, coconut, oil palm, safflower, castor-oil plant, Calendula, peanut, cocoa bean or sunflower. In principle, nonhuman transgenic animals are also suitable as host organisms, for example C. elegans.

Preferred transgenic plants are those which comprise a functional or nonfunctional nucleic acid construct according to the invention or a functional or nonfunctional vector according to the invention. For the purposes of the invention, functional means that the nucleic acids used in the method, alone or in the nucleic acid construct or in the vector, are expressed and a biologically active gene product is produced. For the purposes of the invention, nonfunctional means that the nucleic acids used in the method, alone or in the nucleic acid construct or in the vector are not transcribed or not expressed and/or that a biologically inactive gene product is produced. In this sense, what are known as antisense RNAs are also nonfunctional nucleic acids or, upon insertion into the nucleic acid construct or the vector, a nonfunctional nucleic acid construct or nonfunctional vector. To generate transgenic organisms, preferably plants, both the nucleic acid construct according to the invention and the vector according to the invention can be used advantageously.

For the purposes of the invention, transgenic/recombinantly is to be understood as meaning that the nucleic acids used in the method are not at their natural place in the genome of an organism, it being possible for the nucleic acids to be expressed homologously or heterologously. However, transgenic/recombinantly also means that the nucleic acids according to the invention are at their natural position in the genome of an organism, but that the sequence has been modified compared with the natural sequence and/or that the regulatory sequences of the natural sequences have been modified. Preferably, transgenic/recombinantly is to be understood as meaning the expression of the nucleic acids at a non-natural position in the genome, that is to say homologous or, preferably, heterologous expression of the nucleic acids takes place. The same also applies to the nucleic acid construct according to the invention or the vector.

Utilizable host cells are furthermore mentioned in: Goeddel, Gene Expression Technology: Methods in Enzymology 185, Academic Press, San Diego, Calif. (1990).

Expression strains which can be used, for example those which exhibit a lower protease activity, are described in: Gottesman, S., Gene Expression Technology: Methods in Enzymology 185, Academic Press, San Diego, Calif. (1990) 119-128.

Furthermore, the invention also encompasses the use of the nucleic acids according to the invention, for example of the nucleotide sequences stated in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 for generating genetically modified plants which comprise modified proteins of the proteins encoded by SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 which have a very much lower interaction with the herbicide or whose activity is not interfered with by the herbicide.

The nucleic acids used in the method according to the invention, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51, the sequences which have been derived from them on the basis of the degeneracy of the genetic code and their derivatives were identified from a population of transgenic plants, which population has, on the one hand, been transformed by means of Agrobacterium and, while performing this process, novel DNA had been integrated randomly in the chromosome. Backcrosses finally allowed plants to be isolated which contain the identified nucleic acids on both homologous chromosomes. These plants are lethal, which is why they die either as early as during the embryonic stage or else during the seedling stage. No homozygous lines were obtained. Moreover, these plants have been identified during the screening process as lines which segregate for lethal mutations. As the result of the homozygous state of the integration of the novel DNA, these plants show severely impaired growth and/or development. It can be assumed that this impaired growth and development can be attributed to the fact that the newly inserted DNA has integrated into genes which are important for growth and development, thus limiting or blocking their biological function in the homozygous state. This means that these genes and the sequences which have been derived on the basis of the degeneracy of the genetic code and their derivatives encode proteins which, analogously for those described in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 constitute suitable target proteins for herbicides to be newly developed.

In an advantageous embodiment, the stated nucleic acids are overexpressed and the following process steps are advantageously carried out in order to generate modified proteins:
a) expression, in a heterologous system, for example a microorganism such as a bacterium of the genus Escherichia, such as E. coli XL1-Red, or in a cell-free system, of the proteins encoded by the nucleic acid sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 or by a nucleic acid sequence which can be derived on the basis of the degeneracy of the genetic code by backtranslating the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 or of proteins encoded by derivatives or fragments of the nucleic acid sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 which encode polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which have at least 50%, 60%, preferably 70%, 80%, 90% or more homology at the amino acid level,
b) randomized or directed mutagenesis of the protein by modification of the nucleic acid,
c) measuring the interaction or the biological activity of the modified protein with the herbicide, or in the presence of the herbicide,
d) identification of derivatives of the protein which exhibit a lesser degree of interaction or a biological activity which has been affected by a lesser degree,
e) testing the biological activity of the protein following application of the herbicide.

The resulting modified protein, or the modified nucleic acid, for example of the sequences stated under SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 and the other sequences according to the invention which are described above, for example derivatives and fragments, for example from other plants are advantageously transferred into an organism, advantageously into a plant, preferably plant cells.

A further embodiment of the invention is a method for generating modified gene products encoded by the nucleic acid sequences, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 according to the invention and described herein, which comprises the following process steps:
a) expression of the proteins encoded by SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 or their derivatives or fragments, for example from other plants, in a heterologous system or in a cell-free system
b) randomized or directed mutagenesis of the protein by modification of the nucleic acid,
c) measuring the interaction of the modified gene product with the herbicide, or the biological activity of the modified gene product in the presence of the herbicide,
d) identification of derivatives of the protein which exhibit a lesser degree of interaction or an activity which has been affected by a lesser degree,
e) testing the biological activity of the protein following application of the herbicide,
f) selection of the nucleic acid sequences which, or whose gene products, show a modified biological activity with regard to the herbicide, preferably a reduced inhibition by the herbicide or a lesser degree of interaction with the herbicide.

The sequences selected by the above-described process can advantageously be introduced into an organism. Therefore, the invention furthermore relates to an organism generated by this method, the organism preferably being a plant. The method is also suitable for the gene expression of the abovementioned biologically active derivatives and fragments.

Subsequently, intact plants are regenerated and the resistance to the herbicide is tested in intact plants.

Modified proteins and/or nucleic acids which, in plants, can mediate resistance to herbicides can also be generated from the sequences according to the invention which are described herein, in particular from the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 or their derivatives from other plants via what is known as site-directed mutagenesis. For example, the stability and/or enzymatic activity of enzymes or the properties such as the binding of low-molecular-weight compounds with less than 1000 molecular weight can be modified in a targeted fashion and advantageously reduced by means of this mutagenesis. Advantageously, the molecular weight of the compounds should amount to less than 900 Daltons, preferably less than 800, especially preferably less than 700, very especially preferably less than 600 Daltons, preferably with a Ki value of less than 10⁻⁷, advantageously less than 10⁻⁸, preferably less than 10⁻⁹ M. This inhibitory effect should advantageously be attributable to a specific inhibition of the biological activity of the nucleic acids according to the invention and/or of the proteins encoded by these nucleic acids, that is to say no inhibition, by these low-molecular-weight substances, of further, closely related nucleic acids and/or of the proteins encoded by them should take place. Moreover, the low-molecular-weight substances should advantageously have a molecular weight of greater than 50 Daltons, preferably greater than 100 Daltons, especially preferably greater than 150 Daltons, very especially preferably greater than 200 Daltons. The low-molecular-weight substances should advantageously have less than three hydroxyl groups on a carbon-atom-comprising ring. Furthermore, no free acid or lactone group(s) and no phosphate group and not more than one amino group should be present in the molecule. Bases such as adenosin are also less preferred in the molecule. Also, the stability and/or enzymatic activity of enzymes, or the properties such as binding of proteins or antisense RNA, can be improved or modified in a highly targeted fashion in this way.

Moreover, modifications may be achieved by the PCR method described by Spee et al. (Nucleic Acids Research, Vol. 21, No. 3, 1993: 777-78), using dITP for the random mutagenesis, or by the further improved method of Rellos et al. (Protein Expr. Purif., 5, 1994: 270-277).

A further possibility of generating these modified proteins and/or nucleic acids is the in vitro recombination technique described by Stemmer et al. (Proc. Natl. Acad. Sci. USA, Vol. 91, 1994: 10747-10751) for molecular evolution or the combination of the PCR and recombination method, which has been described by Moore et al. (Nature Biotechnology Vol. 14, 1996: 458-467).

A further way of mutating nucleic acids and proteins is described by Greener et al. in Methods in Molecular Biology (Vol. 57, 1996: 375-385). EP-A-0 909 821 describes a method of modifying proteins using the microorganism E. coli XL-1 Red. Upon replication, this microorganism generates mutations in the introduced nucleic acids and thus leads to a modification of the genetic information. Advantageous nucleic acids and the proteins encoded by them and vice versa can be identified readily via isolation of the modified nucleic acids or the modified proteins and carrying out of resistance testing. After introduction into plants, they can manifest resistance therein and thus lead to resistance to the herbicides.

Further methods of mutagenesis and selection are, for example, methods such as the in vivo mutagenesis of seeds or pollen and selection of resistant alleles in the presence of the inhibitors according to the invention, followed by the genetic and molecular identification of the modified, resistant allele. Furthermore, the mutagenesis and selection of resistances in cell culture by growing the culture in the presence of successively increasing concentrations of the inhibitors according to the invention. In doing so, the increase in the spontaneous mutation rate by chemical/physical mutagenic treatment may be exploited. As described above, modified genes may also be isolated using microorganisms which have an endogenous or recombinant activity of the proteins encoded by the nucleic acids used in the method according to the invention, which microorganisms are sensitive to the inhibitors identified in accordance with the invention. Growing the microorganisms on media with increasing concentrations of inhibitors according to the invention permits the selection and evolution of resistant variants of the targets according to the invention. The frequency of the mutations, in turn, can be increased by mutagenic treatments.

In addition, methods are available for the targeted modifications of nucleic acids (Zhu et al. Proc. Natl. Acad. Sci. USA, Vol. 96, 8768-8773 and Beethem et al., Proc. Natl. Acad. Sci. USA, Vol 96, 8774-8778). These methods make it possible to replace, in the proteins, those amino acids which are of importance for binding inhibitors by functionally equivalent amino acids which, however, inhibit the binding of the inhibitor.

The invention therefore furthermore relates to a method of generating nucleotide sequences which encode gene products with a modified biological activity, the biological activity being modified such that an increased activity is present. Increased activity is to be understood as meaning an activity which is increased over the original organism, or over the original gene product, by at least 10%, preferably by at least 30%, especially preferably by at least 50% or 70%, very especially preferably by at least 100%. Moreover, the biological activity may have been modified such that the substances and/or compositions according to the invention no longer, or no longer correctly, bind to the nucleic acid sequences and/or the gene products encoded by them. No longer, or no longer correctly, is to be understood as meaning for the purposes of the invention that the substances bind at least 30% less, preferably at least 50% less, especially preferably at least 70% less, very especially preferably at least 80% less or not at all to the modified nucleic acids and/or gene products in comparison with the original gene product or the original nucleic acids.

Yet a further aspect of the invention therefore relates to a transgenic plant which has been genetically modified by the above-described method according to the invention.

Genetically modified transgenic plants which are resistant to the substances found in accordance with the methods according to the invention and/or to compositions comprising these substances may also be generated by overexpressing the nucleic acids, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51, used in the methods according to the invention. The invention therefore furthermore relates to a method of generating transgenic plants which are resistant to substances which have been found by a method according to the invention, wherein nucleic acids according to the invention with one of the above-described biological activities, in particular with the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51, are overexpressed in these plants. A similar method is described, for example, in Lermantova et al., Plant Physiol., 122, 2000: 75-83. Naturally, the derivatives and fragments mentioned herein, for example from other plants, which have the desired activity may also be used.

The above-described methods according to the invention for generating resistant plants make possible the development of novel herbicides which have as complete as possible an action which is independent of the plant species (what are known as nonselective herbicides), in combination with the development of useful plants which are resistant to the nonselective herbicide. Useful plants which are resistant to nonselective herbicides have already been described on several occasions. In this context, one can distinguish between several principles for achieving a resistance:
a) Generation of resistance in a plant via mutation methods or recombinant methods by markedly overproducing the protein which acts as target for the herbicide and by the fact that, owing to the large excess of the protein which acts as target for the herbicide, the function exerted by this protein in the cell is retained even after application of the herbicide.
b) Modification of the plant such that a modified version of the protein which acts as target of the herbicide is introduced and that the function of the newly introduced modified protein is not adversely affected by the herbicide.
c) Modification of the plant such that a novel protein/a novel RNA is introduced wherein the chemical structure of the protein or of the nucleic acid, such as of the RNA or the DNA, which structure is responsible for the herbicidal action of the low-molecular-weight substance, is modified so that, owing to the modified structure, a herbicidal action can no longer be developed or the herbicide in the modified plant is inactivated or modified, for example catabolized, not taken up or not transported or transported into the vacuole, and the like, that is to say that the interaction of the herbicide with the target can no longer take place.
d) The function of the target is replaced by a novel nucleic acid introduced into the plant, for example a gene, the nucleic acid encoding a gene product whose function is inhibited to a lesser degree or not at all by the herbicidal substance. In this manner, for example, what is known as an alternative pathway is created.
e) The function of the target is taken over by another gene which is present in the plant or introduced into the plant, or by its gene product.

The present invention therefore furthermore relates to the use of plants comprising the genes affected by T-DNA insertion which have the nucleic acid sequences used in the method according to the invention, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 or the other sequences mentioned, for example fragments and derivatives, for example from other plants, for the development of novel herbicides. The skilled worker is familiar with alternative methods of identifying homologous nucleic acids, for example in other plants with similar sequences, such as, for example, using transposons. The present invention therefore also relates to the use of alternative insertion mutagenesis methods for inserting foreign nucleic acid into the nucleic acid sequences according to the invention and described herein, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 into sequences derived from these sequences on the basis of the genetic code and/or their derivatives or fragments, for example from other plants.

The invention therefore furthermore relates to substances as described above, identified by the methods according to the invention, the substance being a compound, advantageously a low-molecular-weight compound with less than 1000 molecular weight, advantageously less than 900 daltons, preferably less than 800 daltons, especially preferably less than 700 daltons, very especially preferably less than 600 daltons, advantageously with a Ki value of less than 10⁻⁷, advantageously less than 10⁻⁸, preferably less than 10⁻⁹ M, advantageously, this inhibitory effect should be attributable to a specific inhibition of the biological activity of the nucleic acids according to the invention and/or of the proteins encoded by these nucleic acids, i.e. no inhibition, by these low-molecular-weight substances, of further, closely related nucleic acids and/or of the proteins encoded by these nucleic acids should take place. Moreover, the low-molecular-weight substances should advantageously have a molecular weight of greater than 50 daltons, preferably greater than 100 daltons, especially preferably greater than 150 daltons, very especially preferably greater than 200 daltons. Advantageously, the low-molecular-weight substances should have fewer than three hydroxyl groups on a carbon-atom-comprising ring. Furthermore, no free acid or lactone group(s) and no phosphate group and not more than one amino group should also be present in the molecule. Bases such as adenosin in the molecule are also less preferred. The substances can advantageously also be a proteinogenic substance, such as an antibody, or an antisense RNA.

A further embodiment of the invention are substances which have been identified by the methods according to the invention described hereinabove, the substances being an antibody to the protein encoded by the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51, or derivatives or fragments of this protein.

The antibodies can also bind several of the sequences mentioned, as long as the binding is specific, i.e. can be identified or tested using the abovementioned methods.

These substances are advantageously distinguished by their herbicidal action which can be identified by means of the above-described methods.

The invention furthermore relates to compositions comprising a herbicidally active amount of at least one substance identified by one of the methods according to the invention or of an antagonist identified by a method according to the invention, and at least one inert liquid and/or solid carrier and, if appropriate, at least one surface-active substance.

A further embodiment are compositions comprising a growth-regulatory amount of at least one substance identified by the methods according to the invention or of an antagonist identified by a method according to the invention, and at least one inert liquid and/or solid carrier and, if appropriate, at least one surface-active substance.

These substances or compositions according to the invention with their herbicidal action can be used as defoliants, desiccants, haulm killers and, in particular, as weed killers. Weeds are to be understood as meaning, in the broadest sense, all plants which grow in locations where they are undesired. Whether the substances or active ingredients found with the aid of the methods according to the invention act as nonselective or selective herbicides depends, inter alia, on the amount used, their selectivity and other factors. For example, the substances can be used against the following weeds:

Dicotyledonous weeds of the genera:

Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Taraxacum.

Monocotyledonous weeds of the genera:

Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, Apera.

Depending on the application method in question, the substances identified in the method according to the invention, or compositions comprising them, may advantageously also be employed in a further number of crop plants for eliminating undesired plants. Examples of suitable crops are: Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera, Zea mays.

The substances found by the method according to the invention can also be used advantageously in crops which tolerate the action of herbicides owing to breeding, including recombinant methods.

The substances according to the invention, or the herbicidal compositions comprising them, can be applied, for example, in the form of directly sprayable aqueous solutions, powders, suspensions, also highly concentrated aqueous, oily or other suspensions or dispersions, emulsions, oil dispersions, pastes, dusts, materials for spreading or granules by means of spraying, atomizing, dusting, spreading or pouring. The use forms depend on the intended purposes; in any case, they should guarantee the finest possible distribution of the active ingredients according to the invention.

Suitable inert liquid and/or solid carriers are liquid additives such as mineral oil fractions of medium to high boiling point, such as kerosene or diesel oil, furthermore coal tar oils and oils of vegetable or animal origin, aliphatic, cyclic and aromatic hydrocarbons, for example paraffin, tetrahydronaphthalene, alkylated naphthalenes or their derivatives, alkylated benzenes or their derivatives, alcohols such as methanol, ethanol, propanol, butanol, cyclohexanol, ketones such as cyclohexanone or strongly polar solvents, for example amines such as N-methylpyrrolidone or water.

Further advantageous embodiments of the substances and/or compositions according to the invention are aqueous use forms such as emulsion concentrates, suspensions, pastes, wettable powders or water-dispersible granules, which can be prepared, for example, by adding water. To prepare emulsions, pastes or oil dispersions, the substances and/or compositions, what are known as the substrates, as such or dissolved in an oil or solvent, may be homogenized in water by means of wetter, adhesive, dispersant or emulsifier. However, concentrates composed of active substance, wetter, adhesive, dispersant or emulsifier and, if appropriate, solvent or oil may also be prepared, and these concentrates are suitable for dilution with water.

Suitable surface-active substances are the alkali metal salts, alkaline earth metal salts and ammonium salts of aromatic sulfonic acids, for example lignosulfonic acid, phenolsulfonic acid, naphthalenesulfonic acid and dibutylnaphthalenesulfonic acid, and of fatty acids, alkylsulfonates and alkylarylsulfonates, alkylsulfates, lauryl ether sulfates and fatty alcohol sulfates, and salts of sulfated hexa-, hepta- and octadecanols, and of fatty alcohol glycol ether, condensates of sulfonated naphthalene, and its derivatives with formaldehyde, condensates of naphthalene or of the naphthalenesulfonic acids with phenol and formaldehyde, polyoxyethylene octylphenyl ether, ethoxylated isooctylphenol, octylphenol or nonylphenol, alkylphenyl polyglycol ethers, tributylphenyl polyglycol ethers, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignin-sulfite waste liquors or methylcellulose.

Powders, materials for spreading and dusts can be prepared advantageously as solid carriers by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas and products of vegetable origin such as cereal meal, tree bark meal, wood meal and nutshell meal, cellulose powders or other solid carriers.

The concentrations of the substances and/or compositions according to the invention in the ready-to-use preparations can be varied within wide ranges. In general, the formulations comprise 0.001 to 98% by weight, preferably 0.01 to 95% by weight, of at least one active ingredient. In this context, the active ingredients are employed in a purity of 90% to 100%, preferably 95% to 100% (according to NMR spectrum).

The herbicidal compositions or the substances can be applied pre- or post-emergence. If the active ingredients are less well tolerated by specific crop plants, application techniques may be used in which the herbicidal compositions or substances are sprayed, with the aid of the spraying apparatus, in such a way that coming into contact with the leaves of the sensitive crop plants is avoided as far as possible, while the active ingredients reach the leaves of undesired plants which grow underneath, or the bare soil surface (post-directed, lay-by).

To widen the spectrum of action and to achieve synergistic effects, the substances and/or compositions according to the invention may be mixed with a large number of representatives of other groups of herbicidal or growth-regulatory active ingredients and applied concomitantly. Suitable examples of components in mixtures are 1,2,4-thiadiazoles, 1,3,4-thiadiazoles, amides, aminophosphoric acid and its derivatives, aminotriazoles, anilides, (het)-aryloxyalkanoic acids and their derivatives, benzoic acid and its derivatives, benzothiadiazinones, 2-aroyl-1,3-cyclohexanediones, hetaryl aryl ketones, benzylisoxazolidinones, meta-CF₃-phenyl derivatives, carbamates, quinolinic acid and its derivatives, chloroacetanilides, cyclohexane-1,3-dione derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- or heteroaryloxyphenoxypropionic esters, phenylacetic acid and its derivatives, phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides, uracils.

Moreover, it may be useful to apply the substances and/or compositions according to the invention, alone or in combination with other herbicides, as a joint mixture together with other crop protection agents, for example with agents for controlling pests or phytopathogenic fungi or bacteria. Also of interest is the miscibility with mineral salt solutions which are employed for alleviating nutritional and trace element deficiencies. Nonphytotoxic oils and oil concentrates may also be added.

Depending on the intended aim of the control measures, the season, the target plants and the growth stage, the application rates of active ingredient (=substance and/or composition) are from 0.001 to 3.0, preferably 0.01 to 1.0, kg of active substance per ha.

The invention furthermore relates to the use of a substance identified by one of the methods according to the invention or of a composition comprising the substances as herbicide or for regulating the growth of plants.

Moreover, the invention relates to a kit encompassing the nucleic acid construct according to the invention, the substances according to the invention, for example the antibody according to the invention, the antisense nucleic acid molecule according to the invention and/or an antagonist and/or a herbicidal substance identified in accordance with the methods according to the invention, and the composition described hereinbelow.

The invention furthermore relates to a composition comprising the substance according to the invention, the antibody according to the invention, the antisense nucleic acid construct according to the invention and/or an antagonist according to the invention and/or a substance according to the invention identified by a method according to the invention.

The invention is illustrated in greater detail by the examples which follow, which should not be taken as limiting.

### EXAMPLES

a) Molecular-Biological Methods

Molecular-biological methods as employed herein are those of the prior art and are described in various references such as, for example, Sambrook et al., Molecular Cloning, eds., Cold Spring Harbor Laboratory Press, Cold Spring Harbor, N.Y. (1989), Reiter et al., Methods in Arabidopsis Research, World Scientific Press (1992), Schultz et al., Plant Molecular Biology Manual, Kluwer Academic Publishers (1998) and Martinez-Zapater and Salinas, Methods in Molecular Biology, Vol. 82: Arabidopsis Protocols eds., Humana Press Inc., Totowa, N.J. These references describe the customary standard methods for the production, identification and cloning of mutants caused by T-DNA insertions. In addition, a further customary method for the identification of insertion sites as was described, for example, by Spertini et al., Biotechniques 27: 308-314 (1999), was resorted to. The sequencing was carried out by DNA LandMarks Inc., Quebec, Canada.
   b) Materials
Unless otherwise specified in the text, the chemicals used were obtained in analytical-grade quality from Fluka (Neu-Ulm), Merck (Darmstadt), Roth (Karlsruhe), Serva (Heidelberg) and Sigma (Deideshofen). Solutions were prepared using pure, pyrogen-free water, obtained from an ion-exchange system by TKA (Niederelbert). Restriction nucleases, DNA-modifying enzymes and molecular biology kits and oligonucleotides were obtained from Amersham Pharmacia (Freiburg), Biometra (Göttingen), Dynal (Hamburg), Gibco-BRL (Gaithersburg, Md., USA), Invitrogen (Groningen, Netherlands), MBI Fermentas (St. Leon Rot), New England Biolabs (Schwalbach, Taunus), Novagen (Madison, Wis., USA), Qiagen (Hilden), Roche Diagnostics (Mannheim), Stratagene (Amsterdam, Netherlands), TTB-Molbiol (Berlin). Unless otherwise specified, the products were employed in accordance with the manufacturers' instructions.

### Example 1

### Generation of a KO Population and Identification of Lines which Segregate for Lethal Mutation

Starting from the basic structure of the pPZP vectors [Hajukiewicz, P. et al., (1994) The small, versatile pPZP family of Agrobacterium binary vectors for plant transformation. Plant Mol. Biol. 25, 989-994], a modified binary vector which comprised the kanamycin resistance gene for the selection in bacteria was constructed. Only one selection cassette consisting of the resistance gene for Clearfield resistance (imidazolinone or AHAS resistance) under the control of the constitutive promoter mas1 (Velten et al., 1984, EMBO J. 3, 2723-2730; Mengiste, Amedeo and Paszkowski, 1997, Plant J., 12, 945-948.) was present between the left and the right T-DNA border. As an alternative, other resistance genes such as the hebicide resistance genes such as the phosphinothricin (=bar resistance), the methionine sulfoximine, the sulfonylurea (=ilv resistance, ind S. cerevisiae ilv2) or the phenoxyphenoxy herbicide resistance genes (=ACCase resistance) or genes for resistance to antibiotics may be used. Also, the skilled worker is familiar with other constitutive promoters which can be used instead of the mas1′ promoter used, such as the 34S, the 35S or the ubiquitin promoter from parsley. The skilled worker is familiar with the various vectors which can be used for the transformation of Arabidopsis by means of Agrobacterium. A detailed description of the vectors which can be employed and of agrobacterial strains can be found in Hellens et al., (Trends in Plant Science, 2000, Vol 5, 446-451). The plasmids were transformed into agrobacteria, in the present case the Agrobacterium tumefaciens strain GV3101 pMP90 (Koncz and Schell, 1986 Mol. Gen. Genet. 204:383-396), by means of a heat-shock protocol. Transformed bacterial colonies were grown for 2 days at 28° C. on YEP medium comprising the antibiotic in question. These agrobacteria were then employed for the transformations of a large number of Arabidopsis ecotype C24 plants (Nottingham Arabidopsis Stock Centre, UK; NASC Stock N906), the procedure being as described in a modified version of the in-planta transformation method (Bechtold, N., Ellis, J., Pelletier, G. 1993. In planta Agrobacterium mediated gene transfer by infiltration of Arabidopsis thaliana plants, C.R. Acad. Sci. Paris. 316:1194-1199; Clough, J C and Bent, A F. 1998 Floral dip: a simplified method for Agrobacterium-mediated transformation of Arabidopsis thaliana, Plant J. 16:735-743). Transformed plants were selected by means of the selection agent, resistance to which being conferred by the resistance gene encoded on the T-DNA.

Approximately 100 to 200 seeds (T2) of these transformed plants were plated on agar plates with selection agent. These plates were stratified for 2 days at 4° C. and incubated for approximately 7 to 10 days at 20° C. under continuous light. Thereafter, the number of seedlings which were resistant and sensitive, respectively, to the selection agent was determined. Moreover, the number of unpigmented plants (albinos) was determined, if appropriate. Owing to their color, these plants were unambiguously different from the sensitive seedlings. Only those lines which obviously segregated for an insertion site, i.e. in which approximately a third to a quarter of the plants showed sensitivity to the selection and in which very close coupling, i.e. a cosegregation between the resistance-conferring T-DNA and the mutation generating the phenotype, was found, were retained for future studies. Such a very close coupling between the T-DNA and the mutation existed when a numerical ratio of 2:1 between resistant and sensitive seedlings was found. This numeric ratio, which differs from a normal 3:1 segregation for an insertion site, only occurs when the homozygously-resistant plants are absent quantitatively, either because they already die at the embryonic stage or do not develop, or else because they manifest an albino phenotype. Accordingly it is highly likely that insertion of the T-DNA at the respective site in the genome is the cause for the mutation which is lethal for the embryo, or the albino mutation. Accordingly, the essential gene can be identified by identifying the insertion site and the gene present at this site.

### Example 2

### Molecular Analysis of Lines with Phenotype which is Lethal for the Embryo or for Albinos

Genomic DNA was isolated by means of standard methods (either columns from Qiagen, Hilden, Germany, or Phytopure Kit from Amersham Pharmacia, Freiburg, Germany) from approximately 50 mg of leaf material of the selected lines which segregated for a mutation which is lethal for albinos or for the embryo and for which cosegregation between T-DNA and mutation was identified. The amplification of the insertion site of the T-DNA was carried out using a modified version of the adaptor PCR method as published by Spertini D, Beliveau C. and Bellemare, 1999, Biotechniques, 27, 308-314. Approximately in each case 50 to 100 ng of the genomic DNA were digested in parallel with the restriction enzymes MunI, BglII, BspI (=Bsp119I), PspI (=Psp1406I) and SpeI and ligated with an adaptor which consisted of annealed oligos 5′CTMTACGACTCACTATAGGGCTCGAGCGGCCGGGCAGGT-3′ and 5′NN(2-4)ACCTGCCCM-3′, with 5′NN₍₂₋₄₎ representing the overhang matching the enzyme in question. One μl of this genomic DNA, which had been provided with adaptors, was employed for an amplification of the T-DNA-flanking sequences using an adaptor-specific (5′-GGATCCTMTACGACTCACTATAGGGC-3′) and in each case a gene-specific primer for each border. The skilled worker is familiar with the way in which gene-specific primers for the T-DNA used for the transformation of plants are designed and synthesized. The PCR was carried out under standard conditions for 7 cycles at an annealing temperature of 72° C. and for 32 cycles at an annealing temperature of 65° C. in a reaction volume of 25 μl. The amplificate obtained was diluted 1:50 in H₂O, and one μl of this dilution was employed in a second amplification step (5 cycles at an annealing temperature of 67° C. and 28 cycles at an annealing temperature of 60° C.). To this end, “nested” primers, i.e. primers located further inside the PCR product, were employed, whereby the specificity and selectivity of the amplification were increased. An aliquot of the amplificate obtained in the 50 μl of reaction volume was analyzed by gel electrophoresis. In each case, one or more specific PCR products for the left and/or the right T-DNA were obtained. The products were purified by means of standard methods (Qiagen, Hilden) and sequenced with the aid of further T-DNA-specific primers. The insertion site of the T-DNA in the genome was determined in each case by a Blast alignment (BLASTN, Altschul, et al., 1990, J Mol. Biol. 215:403-410) of the isolated sequence with the published genome sequences of Arabidopsis (The Arabidopsis Genome Initiative, 2000, Nature, 408:796-815). Since these sequences are available in annotated form in a variety of databases with which the skilled worker is familiar, it was also possible to determine the ORFs which had been inactivated in each case. The successful identification of an inactivated ORF was verified by a PCR reaction using a primer with specificity for the derived flanking sequence and one primer with specificity for the T-DNA. Obtaining the PCR product of the expected size which was specific for the line in question confirmed the successful identification of the insertion site of the T-DNA.

### Example 3

### Identification and Analysis of Line 303317, which Segregates a Lethal Mutation

Line 303317 was identified as described above (Examples 1 and 2) as a line which segregates for a mutation which is lethal for the seedling. The accurate determination of the segregation revealed that 25% of the progeny showed the albino phenotype, 25% of the progeny sensitivity to the selection and 50% of the progeny resistance to the selection. This segregation ratio is expected when exclusvely the homozygously-resistant seedlings show the phenotype, which is why the T-DNA insertion is coupled very closely to the lethal mutation. The coupling was furthermore checked in a cosegregation analysis. To this end, the progeny of 40 wild-type resistance plants of line 303317 was analyzed. Again, albinos were found in the progeny in all cases. This fact allows the conclusion that the resistance-conferring T-DNA insertion and the mutation are always inherited together and therefore coincide (with a high degree of probability). The molecular-biological analysis was carried out as described in Example 1. For line 303317, a 1400 bp fragment for the enzyme MunI was identified for the left T-DNA border. Obtaining the PCR product of the predicted size, which is specific for this line, confirmed the successful identification of the insertion site of the T-DNA. Blast analysis of the isolated sequence (BLASTN, Altschul et al., 1990) J Mol. Biol. 215:403-410) demonstrated the insertion of the T-DNA in position 6628 of the BAC clone ATF2809 with the Accession Number AL137080. According to the annotation of this region, the integration has taken place in an ORF (F2809.40, SEQ ID NO: 1) which has similarity to the translation releasing factor RF-2 from Synechocystis sp. (PIR:S76448). Moreover, the protein (SEQ ID NO: 2) has an araC family signature. The successful identification of the insertion site and of the inactivated ORFs was verified by PCR reaction with a primer with specificity for the derived flanking sequence and a primer with specificity for the T-DNA.

### Example 4

### Identification and Analysis of the Lines 304149, 120701, 126548, 127023, 127235, 218031, 171042, KO-T3-02-33338-3, KO-T3-02-33885-2 and KO-T3-02-35172-2 which Segregate for a Lethal Mutation

Analogously to the above Examples 1 to 4, the clones 304149, 120701, 126548, 127023, 127235, 218031, 171042, KO-T3-02-33338-3, KO-T3-02-33885-2 and KO-T3-02-35172-2 were identified as the lines which segregate for mutations which are lethal for the embryo or the seedling. The segregation was in all lines as described in Example 3 or analogously to Example 3 for mutations which are lethal for the embryo. However, the mutation which is lethal for the embryo leads to the plants which are homozygous for the mutation interrupting their development as early as during the embryonic stage and thus do not germinate at all. Accordingly, the numeric ratio shifts to one third of plants which are sensitive and two thirds of plants which are resistant to the selection. The molecular-biological work and analyses were carried out as described under Examples 1 to 3.

Line 304149 segregates for a mutation which is lethal for albinos and which cosegregates with the resistance marker and thus the T-DNA. For line 304149, a 750 bp fragment was identified for the enzyme MunI, a 300 bp fragment for the enzyme Psp1406I/Bsp119I and a 950 bp fragment for the enzyme SpeI, in each case for the left T-DNA border. For the right T-DNA border, a 300 bp fragment was identified using the enzyme SpeI. Sequencing these fragments revealed the same insertion site. The T-DNA is inserted on chromosome 5 in position 35398 of the P1 clone MSH12, Accession AB006704. Owing to the insertion 110 bp upstream of the start codon of the ORF MSH12.9, it is highly likely that transcription is prevented or transcript stability reduced, and the functionality of the ORF is thus reduced or completely destroyed. This ORF MSH12.9 encodes a cobalamin synthesis protein.

Line 120701 segregates for a mutation which is lethal for albinos and which cosegregates with the resistance marker and thus the T-DNA. For line 120701, a 500 bp fragment for the enzyme BglII was identified for the left T-DNA border. The T-DNA is inserted on chromsome 4 in position 55170 of the BAC clone ATT25K17, Accession AL049171. Owing to the insertion within the coding region, the ORF T25K17.110 is interrupted and thus inactivated. This ORF T25K17.110 encodes an arginyl-tRNA synthetase. This ORF comprises the EST: gb:AA404880, T76307.

Line 126548 segregates for a mutation which is lethal for the embryo and which cosegregates with the resistance marker and thus the T-DNA. For line 126548, a 1000 bp fragment for the enzymes Psp1406I/Bsp119I was identified for the left T-DNA border. For the right T-DNA border, a 900 bp fragment was identified with the enzymes Psp1406I/Bsp119I and a 300 bp fragment with the enzyme BglII. Sequencing of all PCR products demonstrated insertion of the T-DNA at the same location in the genome. The T-DNA is inserted on chromsome 4 in position 36872 of the Bac clone ATF17A8, Accession AL049482. Owing to the insertion within the coding region, the ORF F17A8.80 is interrupted and thus inactivated. This ORF F17A8.80 encodes a putative protein similarity to a murine (Mus musculus) RNA helicase, PIR2:184741.

Line 127023 segregates for a mutation which is lethal for the embryo and which cosegregates with the resistance marker and thus the T-DNA. For line 127023, a 350 bp fragment for the enzyme BglII and a 900 bp fragment for the enzymes Psp1406I/Bsp119I were identified, in each case for the left T-DNA border. After sequencing, the two fragments identified the identical insertion site. The T-DNA is inserted on chromsome 4 in position 61403 of the BAC clone ATT19P19, Accession AL022605. Owing to this insertion, the ORF A-T4g39780 is interrupted and thus inactivated. This ORF AT4g39780 encodes a putative protein with simiilarity to the Arabidopsis thaliana protein RAP 2.4, which comprises the AP2 domain. Moreover, this ORF comprises the ESTs gb:T46584 and AA394543.

Line 127235 segregates for a mutation which is lethal for the embryo and which cosegregates with the resistance marker and thus the T-DNA. For line 127235, a 1600 bp fragment for the enzyme MunI was identified for the left T-DNA border. For the right T-DNA border, a 600 bp fragment was identified with the enzyme BglII. After sequencing, the two fragments identified the identical insertion site. The T-DNA is inserted on chromosome 1 in position 10776 of the BAC clone F9K20, Accession AC005679. Owing to this insertion, the ORF F9K20.4 is interrupted and thus inactivated. This ORF F9K20.4 encodes a putative protein with similarity to the gi|1786244 hypothetical 24.9 kD protein in the surA-hepA intergenic region yab0 of the Escherichia coli genome gb|AE000116 and to the hypothetical protein of the YABO family PF|00849. Moreover, the protein encoded by ORF F9K20.4 possesses a conserved pseudouridylate synthase domain, which is involved in the modification of uracil in RNA molecules. Accordingly, the ORF F9K20.4 reveals significant homology with various pseudouridylate synthases in the blastp alignment under standard conditions.

Line 218031 segregates for a mutation which is lethal for albinos and cosegregates with the resistance marker and thus the T-DNA. For line 218031, a 400 bp fragment for the enzyme BglII was identified for the left T-DNA border, and this fragment was subsequently sequenced. The T-DNA is inserted on chromsome 2 in position 11909 of clone F3G5 with the Accession AC005896. Owing to the insertion in the coding region, the ORF At2g37250 is inactivated. This ORF encodes a putative adenylate kinase.

Line 171042 segregates for a mutation which is lethal for albinos and which cosegregates with the resistance marker and thus the T-DNA. For line 171042, a 1600 bp fragment for the enzymes Psp1406I/Bsp119I was identified for the left T-DNA border, and this fragment was subsequently sequenced. The T-DNA is inserted on chromsome 3 in position 97005 of the Bac clone T29H11 with the Accession AL049659. Owing to the insertion in the coding region, the ORF T29H11_{—}270 is inactivated. This ORF T29H11_{—}270 encodes a putative protein with similarity to the pol polyprotein of the equine infectious anemia virus (PIR:GNLJEV).

Line KO-T3-02-33338-3 segregates for a mutation which is lethal for albinos and which cosegregates with the resistance marker and thus the T-DNA. For line KO-T3-02-33338-3, a 624 bp fragment for the enzyme MunI was identified for the left T-DNA border, and this fragment was subsequently sequenced. The T-DNA is inserted on chromosome 5 in position 39500 of the P1 clone MJE7 with the Accession AB020745. Owing to the insertion 64 base pairs downstream of the stop codon of the ORF MEJ7.11, the transcript of this ORF is probably modified and thus transcript stability reduced. Accordingly, it can be assumed that the gene function for this ORF is reduced or blocked entirely. ORF MEF7.11 encodes an unknown protein.

Line KO-T3-02-33885-2 segregates for a mutation which is lethal for albinos and which cosegregates with the resistance marker and thus the T-DNA. For line KO-T3-02-33885-2, a 450 bp fragment for the enzymes Psp1406I/Bsp119I has been identified for the left T-DNA border. For the right T-DNA border, a 650 bp fragment was identified with the enzymes Psp1406I/Bsp119I. After sequencing, the two fragments identified the identical insertion site. The T-DNA is inserted on chromosome 1 in position 76356 of the Bac clone F14G9 with the Accession AC069159. Owing to the insertion in the coding region of the ORF F14G9.26, this ORF is inactivated in this line. ORF F14G9.26 encodes an unknown protein.

Line KO-T3-02-35172-2 segregates for a mutation which is lethal for albinos and which cosegregates with the resistance marker and thus the T-DNA. For line KO-T3-02-35172-2, a 700 bp fragment for the enzyme MunI was identified for the right T-DNA border and this fragment was subsequently sequenced. The T-DNA is inserted on chromsome 5 in position 24422 of the P1 clone MAB16 with the Accession AB018112. Owing to this insertion 87 bp upstream of the ORF MAB16.6, the transcription of this ORF is most likely blocked and the gene thus silenced. The ORF MAB16.6 encodes a protein which only shows homology with other unknown proteins.

### Example 5

### Identification and Analysis of Lines 305861, 303814, KO-T3-02-132241, KO-T3-02-15114-2, KO-T3-02-18601-1 and 304143, which Segregate for Mutations which are Lethal for Albinos

Analogously to the above Examples 1 to 4, the clones 305861, 303814, KO-T3-02-132241, KO-T3-02-15114-2, KO-T3-02-18601-1 and 304143 were identified as lines which segregate for mutations which are lethal for albinos. The segregation was in all lines as described in Example 3. The molecular-biological work and analyses were carried out as described under Examples 1 to 3.

Line 305861 segregates for a mutation which is lethal for albinos and cosegregates with the resistance marker and thus the T-DNA. For line 305861, an approximately 1300 bp fragment for the enzyme combination Bgl II was identified for the left T-DNA border. Sequencing this fragment revealed the insertion of the T-DNA in this line at base pair position 16326 of the BAC T7B11, Accession AC007138 on chromosome 4. Owing to the insertion into the open reading frame, the ORF 7B11.6 is interrupted and inactivated. This ORF encodes a preprotein translocase secA precursor protein and is therefore a chloroplastidial SecA protein which is responsible for the transport of proteins across the thylakoid membrane. The insertion of the T-DNA into the abovementioned ORF was verified by means of a control PCR which, using a T-DNA-specific primer and an ORF-specific primer, yielded a fragment of the expected size.

Line 303814 segregates for a mutation which is lethal for albinos and which cosegregates with the resistance marker and thus the T-DNA. For line 303814, an approximately 1300 bp fragment for the enzyme combination Mun I was identified for the left T-DNA border. Sequencing this fragment revealed the insertion of the T-DNA in this line at base pair position 2027 of the BAC F2G19, Accession AC083835 on chromosome 1. Owing to the insertion into the open reading frame, the ORF F2G19.1 is interrupted and inactivated. This ORF encodes a protein with significant homology to the tomato DCL protein, PIR:S71749. Furthermore, the protein has what is known as an HMG signature of the high-mobility-group proteins which are capable of binding to DNA. The insertion of the T-DNA into the abovementioned ORF was verified by means of a control PCR which, using a T-DNA-specific primer and an ORF-specific primer, yielded a fragment of the expected size.

Line KO-T3-02-13224-1 segregates for a mutation which is lethal for albinos and which cosegregates with the resistance marker and thus the T-DNA. For line KO-T3-02-13224-1, an approximately 500 bp fragment for the enzyme combination Bgl II was identified for the left T-DNA border. Sequencing this fragment revealed the insertion of the T-DNA in this line at base pair position 55170 of the BAC T25K17, Accession AL049171 on chromosome 4. Owing to the insertion into the open reading frame, the ORF T25K17.110 is interrupted and inactivated. This ORF encodes an arginine-tRNA ligase. The insertion of the T-DNA into the abovementioned ORF was verified by means of a control PCR which, using a T-DNA-specific primer and an ORF-specific primer, yielded a fragment of the expected size.

Line KO-T3-02-15114-2 segregates for a mutation which is lethal for albinos and which cosegregates with the resistance marker and thus the T-DNA. For line KO-T3-02-15114-2, an approximately 350 bp fragment for the enzyme combination Mun I was identified for the left T-DNA border. Sequencing this fragment revealed the insertion of the T-DNA in this line at base pair position 6984 of the BAC T5N23, Accession AL138650 on chromosome 3. Owing to the insertion into the open reading frame, the ORF T5N23.20 was interrupted and inactivated. This ORF encodes a plastidial glutathione reductase. The insertion of the T-DNA into the abovementioned ORF was verified by means of a control PCR which, using a T-DNA-specific primer and an ORF-specific primer, yielded a fragment of the expected size.

Line KO-T3-02-18601-1 segregates for a mutation which is lethal for albinos and which cosegregates with the resistance marker and thus the T-DNA. For line KO-T3-02-18601-1, an approximately 600 bp fragment for the enzyme combination Bgl II was identified for the right T-DNA border. Sequencing this fragment revealed the insertion of the T-DNA in this line at base pair position 4026 of the BAC F22O13, Accession AC003981 on chromosome 1. Owing to the insertion into the open reading frame, the ORF F22O13.2 is interrupted and inactivated. This ORF encodes a transcription initiation factor sigma homolog, therefore a plant homolog to the sigma subunit of the bacterial RNA polymerase. The insertion of the T-DNA into the abovementioned ORF was verified by means of a control PCR which, using a T-DNA-specific primer and an ORF-specific primer, yielded a fragment of the expected size.

Line 304143 segregates for a mutation which is lethal for albinos and which cosegregates with the resistance marker and thus the T-DNA. For line 304143, an approximately 950 bp fragment for the enzyme Bgl II was identified for the right T-DNA border. Sequencing this fragment revealed the insertion of the T-DNA in this line at base pair position 79156 of the BAC F9013 map mi398, Accession AC006248 on chromosome 2. Owing to the insertion into the promoter, therefore approximately 450 bp upstream of the start codon, the transcription of the ORF At2g15680 is probably prevented and thus the gene function silenced. The ORF At2g15680 encodes a putative calmudulin-like protein. The insertion of the T-DNA into the abovementioned ORF was verified by means of a control PCR which, using a T-DNA-specific primer and an ORF-specific primer, yielded a fragment of the expected size.

### Example 6

### Identification and Analysis of the Lines KO-T3-02-403222-2, KO-T3-02-40309-1, KO-T3-02-40309-2, KO-T4-02-00666-4, KO-T4-02-00666-5, KO-T3-02-41568-2, KO-T3-02-42903-1, KO-T3-02-41395-1 and KO-T3-02-446344, which Segregate for Mutations which are Lethal for Embryos

Analogously to the above Examples 1 to 4, the clones KO-T3-02-403222-2, KO-T3-02-40309-1, KO-T3-02-40309-2, KO-T4-02-00666-4, KO-T4-02-00666-5, KO-T3-02-41568-2, KO-T3-02-42903-1, KO-T3-02-41395-1 and KO-T3-02-446344 were identified as lines which segregate for mutations which are lethal for embryos.

These lines segregate analogously to Example 3, which had been described for lines which are lethal for seedlings. However, the mutation which is lethal for embryos leads to the plants with homozygosity for the mutation interrupting their development as early as during the embryonic stage, and hence do not germinate at all. Accordingly, the numeric ratio shifts to one third of plants which are sensitive and two thirds of plants which are resistant to the selection. The molecular-biological work or analyses were carried out as described under Examples 1 to 3.

Line KO-T3-02-40322-2 segregates for a mutation which is lethal for embryos and which cosegregates with the resistance marker and thus the T-DNA. For line KO-T3-02-40322-2, an approximately 620 bp fragment for the restriction enzyme Mun I was identified for the left T-DNA border by means of adapter PCR. Sequencing this fragment revealed the insertion of the T-DNA in this line at base pair position 5261 of the BAC MPX5, Accession AP002048 on chromosome 3. Owing to the insertion in the promoter region approximately 243 bp upstream of the reading frame, the transcription of the ORF MPX5.1 is prevented and the gene function thus silenced. This ORF encodes a protein with similarity to an unknown protein. The insertion of the T-DNA into the abovementioned ORF was verified by means of a control PCR which, using a T-DNA-specific primer and an ORF-specific primer, yielded a fragment of the expected size.

Line KO-T3-02-40309-1 segregates for a mutation which is lethal for embryos and which cosegregates with the resistance marker and thus the T-DNA. For line KO-T3-02-40309-1, an approximately 900 bp fragment for the enzyme Mun I was identified for the right T-DNA border by means of adapter PCR. Sequencing this fragment revealed the insertion of the T-DNA in this line at base pair position 38553 of the BAC F28O9, Accession AL137080 on chromosome 3. Owing to the insertion in the promoter region approximately 24 bp upstream of the reading frame, the transcription of the ORF F28O9.140 is prevented and the gene function thus silenced. This ORF encodes a protein with high similarity to INT6, a breast-cancer-associated protein, and with similarity to an initiation factor 3 protein. The insertion of the T-DNA into the abovementioned ORF was verified by means of a control PCR which, using a T-DNA-specific primer and an ORF-specific primer, yielded a fragment of the expected size.

Line KO-T3-02-40309-1 segregates for a mutation which is lethal for embryos and which cosegregates with the resistance marker and thus the T-DNA. For line KO-T3-02-40309-1, an approximately 900 bp fragment for the enzyme Mun I was identified for the right T-DNA border by means of adapter PCR. Sequencing this fragment revealed the insertion of the T-DNA in this line at base pair position 38553 of the BAC F28O9, Accession AL137080 on chromosome 3. Owing to the insertion in the promoter region approximately 515 bp upstream of the reading frame, the transcription of the ORF F28O9.150 is prevented and the gene function thus silenced. This ORF encodes a protein with high similarity to the Saccharomyces DNA helicase YGL150c. The insertion of the T-DNA into the abovementioned ORF was verified by means of a control PCR which, using a T-DNA-specific primer and an ORF-specific primer, yielded a fragment of the expected size.

Line KO-T4-02-006664 segregates for a mutation which is lethal for embryos and which cosegregates with the resistance marker and thus the T-DNA. For line KO-T4-02-006664, an approximately 390 bp fragment for the enzyme Bgl II was identified for the left T-DNA border by means of adapter PCR. Sequencing this fragment revealed the insertion of the T-DNA in this line at base pair position 9358 of the BAC MKN22, Accession AB019234 on chromosome 5. Owing to the insertion in the 3′-UTR region, approximately 82 bp downstream of the reading frame, the transcript of the ORF MKN22.2 is most likely destabilized and the gene function thus silenced. This ORF encodes a protein with similarity to an RNA-binding protein. The insertion of the T-DNA into the abovementioned ORF was verified by means of a control PCR which, using a T-DNA-specific primer and an ORF-specific primer, yielded a fragment of the expected size.

Line KO-T4-02-006664 segregates for a mutation which is lethal for embryos and which cosegregates with the resistance marker and thus the T-DNA. For line KO-T4-02-00666-4, an approximately 650 bp fragment for the enzyme Spe I was identified for the left T-DNA border by means of adapter PCR. Sequencing this fragment revealed the insertion of the T-DNA in this line at base pair position 48978 of the BAC MEE6, Accession AB010072 on chromosome 5. Owing to the insertion into the open reading frame, the ORF MEE6.19 is interrupted and inactivated. This ORF encodes a protein with high similarity to an unknown protein. The insertion of the T-DNA into the abovementioned ORF was verified by means of a control PCR which, using a T-DNA-specific primer and an ORF-specific primer, yielded a fragment of the expected size.

Line KO-T3-02-41568-2 segregates for a mutation which is lethal for embryos and which cosegregates with the resistance marker and thus the T-DNA. For line KO-T3-02-41568-2 an approximately 500 bp fragment for the enzyme Bgl II was identified for the right T-DNA border by means of adapter PCR. Sequencing this fragment revealed the insertion of the T-DNA in this line at base pair position 6993 of the BAC T19L18, Accession AC004747 on chromosome 2. Owing to the insertion in the 3′-UTR region, approximately 285 bp downstream of the reading frame, the transcript of the ORF At2g26150 is most probably destabilized and the gene function thereby silenced. This ORF encodes a putative heat shock transcription factor. The insertion of the T-DNA into the abovementioned ORF was verified by means of a control PCR which, using a T-DNA-specific primer and an ORF-specific primer, yielded a fragment of the expected size.

Line KO-T3-02-42903-1 segregates for a mutation which is lethal for embryos and which cosegregates with the resistance marker and thus the T-DNA. For line KO-T3-02-42903-1, an approximately 1300 bp fragment for the degenerate primer ADP3 (5′-WGTGNAGWANCANAGA-3′) was identified for the left T-DNA border by means of TAIL-PCR. Sequencing this fragment revealed the insertion of the T-DNA in this line at base pair position 25933 of the BAC T1E2, Accession AC006929 on chromosome 2. Owing to the insertion into the open reading frame, the ORF At2g28030 is interrupted and inactivated. This ORF encodes a putative chloroplastidial protein which binds to the DNA nucleoid. The insertion of the T-DNA into the abovementioned ORF was verified by means of a control PCR which, using a T-DNA-specific primer and an ORF-specific primer, yielded a fragment of the expected size.

Line KO-T3-02-41395-1 segregates for a mutation which is lethal for embryos and which cosegregates with the resistance marker and thus the T-DNA. For line KO-T3-02-41395-1, an approximately 910 fragment for the enzyme Mun I was identified for the left T-DNA border by means of adapter PCR. Sequencing this fragment revealed the insertion of the T-DNA in this line at base pair position 153501 of the BAC ATCHRIV25, Accession AL161513 on chromosome 4. Owing to the insertion into the gene, the ORF AT4g08990 is interrupted and inactivated. This ORF encodes a protein with similarity to a putative Met2-type cytosine DNA methyltransferase with great similarity to an Arabidopsis thaliana DNA-(cytosine-5-)methyltransferase. The insertion of the T-DNA into the abovementioned ORF was verified by means of a control PCR which, using a T-DNA-specific primer and an ORF-specific primer, yielded a fragment of the expected size.

Line KO-T3-02-44634-4 segregates for a mutation which is lethal for embryos and which cosegregates with the resistance marker and thus the T-DNA. For line KO-T3-02-44634-4, an approximately 800 bp fragment for the degenerate primer ADP8 (5′-NTGCGASWGANWAGAA-3′) was identified for the left T-DNA border by means of TAIL-PCR. Sequencing this fragment revealed the insertion of the T-DNA in this line at base pair position 16225 of the BAC F12B17, Accession AL353995 on chromosome 5. Owing to the insertion into the open reading frame, the ORF F12B17_{—}70 is interrupted and inactivated. This ORF encodes a putative protein with similarity to a postulated Arabidopsis thaliana protein. The insertion of the T-DNA into the abovementioned ORF was verified by means of a control PCR which, using a T-DNA-specific primer and an ORF-specific primer, yielded a fragment of the expected size.

1. A method for identifying herbicidally active substances comprising selecting a substance which reduces or blocks the expression or the activity of the gene product of a nucleic acid or a gene, wherein the nucleic acid or gene comprises:
aa) a nucleic acid sequence with the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51;
bb) a nucleic acid sequence which can be derived from the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 by backtranslation owing to the degeneracy of the genetic code;
cc) a nucleic acid sequence which is a derivative or a fragment of the nucleic acid sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO-45, SEQ ID NO: 47, SEQ ID NO: 49 or SEQ ID NO: 51 and which has at least 60% homology at the nucleic acid level;
dd) a nucleic acid sequence which encodes derivatives or fragments of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24 SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which have at least 50% homology at the amino acid level;
ee) a nucleic acid sequence which encodes a fragment or an epitope of a polypeptide which binds specifically to an antibody, the antibody specifically binding to a polypeptide which is encoded by the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51;
ff) a nucleic acid sequence which encodes a fragment of a nucleic acid shown in aa) and which has a translation releasing factor activity; a cobalamin synthase activity, an arginyl-tRNA synthase activity, an RNA helicase activity, a GTP binding protein activity, a pseudouridylate synthase activity, an adenylate kinase activity, a preprotein translocase secA precursor protein activity, a DCL protein activity, an arginine-tRNA ligase activity, a plastidial glutathione reductase activity, a transcription factor sigma activity, a calmodulin activity, an INT6 activity, a helicase YGL150c activity, an RNA-binding activity, a heat shock transcription factor activity, a chloroplastidial DNA nucleoid binding activity or a Met2-type cytosine DNA methyltransferase activity; and/or
gg) a nucleic acid sequence which encodes derivatives of the polypeptides with the amino acid sequences shown in SEQ ID NO: 2, SEQ ID NO: 4, SEQ ID NO: 6, SEQ ID NO: 9, SEQ ID NO: 10, SEQ ID NO: 12, SEQ ID NO: 14 SEQ ID NO: 16, SEQ ID NO: 18, SEQ ID NO; 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO; 50 or SEQ ID NO: 52 and which has at least 20% homology at the amino acid level and has an equivalent biological activity; or wherein the gene product comprises an amino acid sequence which is encoded by a nucleic acid sequence of aa) to gg).

2. The method of claim 1, wherein the expression or the activity of the nucleic acid or the gene product is reduced or blocked by reducing or blocking the
a) transcription,
b) translation,
c) processing and/or
d) modification
of the nucleic acid sequence or amino acid sequence in claim 1.

3. The method of claim 1, wherein the activity of the nucleic acid or of the protein is reduced or blocked by a low-molecular-weight substance.

4. The method of claim 1, wherein the identification of the substances is carried out in a high-throughput screening (HTS).

5. The method as claimed in one of claims 1 to 4 of claim 1, wherein the selected substances are applied to a plant in order to test the herbicidal activity of the substances and the substances which show herbicidal activity are selected.

6. The method of claim 1, wherein the method is carried out in an organism.

7. The method of claim 6, wherein bacteria, yeasts, fingi or plants are used as the organism.

8. The method of claim 1, wherein the method is carried out in an organism which is a conditional or natural mutant of one of the sequences described in claim 1.

9. A nucleic acid construct comprising a nucleic acid sequence selected from the group consisting of
a) a nucleic acid sequence with the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51;
b) a nucleic acid sequence which can bc derived from the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 by backtranslation owing to the degeneracy of the genetic code;
c) a nucleic acid sequence which is a derivative or a fragment of the nucleic acid sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 and which has at least 60% homology at the nucleic acid level;
d) a nucleic acid sequence which encodes derivatives or fragments of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which have at least 50% homology at the amino acid level;
c) a nucleic acid sequence which encodes a fragment or an epitope of a polypeptide which binds specifically to an antibody, the antibody specifically binding to a polypeptide which is encoded by the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ TD NO: 47, SEQ ID NO: 49 or SEQ ID NO: 51;
f) a nucleic acid sequence which encodes a fragment of a nucleic acid shown in a) and which has a translation releasing factor activity; a cobalamin synthase activity, an arginyl-tRNA synthase activity, an RNA helicase activity, a GTP binding protein activity, a pseudouridylate synthase activity, an adenylate kinase activity, a preprotein translocase secA precursor protein activity, a DCL protein activity, an arginine-tRNA ligase activity, a plastidial glutathione reductase activity, a transcription factor sigma activity, a calmodulin activity, an INT6 activity, a helicase YGL150c activity, an RNA-binding activity, a heat shock transcription factor activity, a chloroplastidial DNA nucleoid binding activity or a Met2-type cytosine DNA methyltransferase activity; and/or
g) a nucleic acid sequence which encodes derivatives of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which has at least 20% homology at the amino acid level and has an equivalent biological activity,
wherein the nucleic acid sequence is linked to one or more regulatory signals.

10. A substance identified by the method of claim 1, wherein the substance has a molecular weight of less than 1000 daltons and more than 50 daltons and a Ki value of less than 10⁻⁷ M.

11. A substance identified by the method of claim 1, wherein the substance is a proteinogenic substance or an antisense RNA.

12. The substance as claimed in claim 11, wherein the substance is an antibody against the protein encoded by a nucleic sequence selected from the group consisting of
a) a nucleic acid sequence with the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51;
b) a nucleic acid sequence which can be derived from the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 by backtranslation owing to the degeneracy of the genetic code;
c) a nucleic acid sequence which is a derivative or a fragment of the nucleic acid sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51 and which has at least 60% homology at the nucleic acid level;
d) a nucleic acid sequence which encodes derivatives or fragments of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which have at least 50% homology at the amino acid level;
c) a nucleic acid sequence which encodes a fragment or an epitope of a polypeptide which binds specifically to an antibody, the antibody specifically binding to a polypeptide which is encoded by the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51;
f) a nucleic acid sequence which encodes a fragment of a nucleic acid shown in a) and which has a translation releasing factor activity; a cobalamin synthase activity, an arginyl-tRNA synthase activity, an RNA helicase activity, a GTP binding protein activity, a pseudouridylate synthase activity, an adenylate kinase activity, a preprotein translocase secA precursor protein activity, a DCL protein activity, an arginine-tRNA ligase activity, a plastidial glutathione reductase activity, a transcription factor sigma activity, a calmodulin activity, an INT6 activity, a helicase YGL150c activity, an RNA-binding activity, a heat shock transcription factor activity, a chloroplastidial DNA nucleoid binding activity or a Met2-type cytosine DNA methyltransferase activity; and/or
g) a nucleic acid sequence which encodes derivatives of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50 or SEQ ID NO: 52 and which has at least 20% homology at the amino acid level and has an equivalent biological activity;
wherein the nucleic acid sequence is linked to one or more regulatory signals.

13. The nucleic acid construct as claimed in claim 9, wherein the nucleic acid construct additionally comprises further nucleic acid sequences.

14. A vector comprising the nucleic acid construct of claim 9.

15. An organism comprising at least one nucleic acid construct as claimed in claim 9.

16. The organism as claimed in claim 15, wherein the organism is a plant, a microorganism or a nonhuman animal.

17. A transgenic plant comprising a functional or nonfunctional nucleic acid construct as claimed in claim 9.

18. (canceled)

19. A method of identifying an antagonist of proteins which are encoded by a nucleic acid sequence as claimed in claim 9 comprising the following steps
i) contacting cells which express the protein, or the protein, with a candidate substance;
ii) testing the biological activity of the protein;
iii) comparing the biological activity of the protein with a standard activity in the absence of the candidate substance, a reduced biological activity of the protein indicating that the candidate substance is an antagonist.

20. The method as claimed in claim 19, wherein the antagonist is applied to a plant to test its herbicidal activity, and those antagonists which show a herbicidal activity are selected.

21. A method of controlling undesired vegetation, which comprises allowing a herbicidally active amount of a substance identified by the method of claim 1 to act on plants and/or their environment.

22. A method for regulating the growth of a plant comprising using an antagonist identified by the method of claim 19.

23. A method for generating modified gene products encoded by the nucleic acid sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO 47, SEQ ID NO 49 or SEQ ID NO: 51, their derivates or fragments as claimed in claim 1, comprising the following steps:
a) expressing the proteins encoded by SEQ ID NO: 1, SEQ ID NO: 3, SEQ ID NO 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49 or SEQ ID NO: 51, their derivatives or fragments as claimed in claim 1 in a heterologous system or in a cell-free system
b) modifying the nucleic acid resulting in randomized or directed mutagenesis of the protein,
c) measuring the interaction of the modified gene product with the herbicide or the biological activity of the modified gene product in the presence of the herbicide,
d) identifying derivatives of the protein which exhibit a lesser degree of interaction or whose activity is less affected,
e) testing the biological activity of the protein following application of the herbicide,
f) selecting nucleic acid sequences which, or whose gene products, show a modified biological activity with regard to the herbicide.

24. The method as claimed in claim 23, wherein the sequences selected are introduced into an organism.

25. A method for generating transgenic plants which are resistant to substances found by the method of claim 1, which comprises overexpressing, in these plants, nucleic acids with the sequences as described in claim 1.

26. An organism generated by the method of claim 24.

27. A composition comprising a herbicidally active amount of at least one substance identified by the method of claim 1 and at least one inert liquid and/or solid carrier.

28. A composition comprising a growth-regulating amount of at least one antagonist identified by the method as claimed in claim 19 and at least one inert liquid and/or solid carrier.

29. A composition comprising the substance of claim 10.

30. A kit comprising the nucleic acid construct of claim 9.


SEQUENCE LISTING (52 sequences)

SEQ ID NO: 1 - DNA, 1230 bp, Arabidopsis thaliana
  CDS (1)..(1230)
atggcggcaaagattattggtggatgctgctcatggcgacgcttttacaggaagagaacatcatctcgatttctgattttctctgttcgagcctctagttccatggatgacatggacaccgtctacaagcaattgggattgttttcactaaagaagaagattaaagatgttgttcttaaggctgagatgtttgcaccggatgctcttgagcttgaagaagagcagtggataaagcaagaagaaacaatgcgttactttgatttatgggatgatcccgctaaatctgatgagattcttctcaaattagctgatcgagctaaagcagtcgattccctcaaagacctcaaatacaaggctgaagaagctaagctgatcatacaattgggtgagatggatgctatagattacagtctctttgagcaagcctatgattcatcactcgatgtaagtagatcgttgcatcactatgagatgtctaagcttcttagggatcaatatgacgctgaaggcgcttgtatgattatcaaatctggatctccaggcgcaaaatctcaggatttgcagatatggacagagcaagttgtaagtatgtatatcaaatgggcagaaaggctaggccaaaacgcgcgggtggctgagaaatgtagtttattgagtaataaaagtggcgtaagttcagccacgatagagtttgaattcgagtttgcttatggttatctcttaggtgagcgaggtgtgcaccgccttatcataagttccacttctaatgaggaatgttcagcgactgttgatatcataccactattcttgagagcatctcctgattttgaagtaaaggaaggtgatttgattgtatcgtatcctgcaaaagaggatcacaaaatagctgagaatatggtttgtatccaccatattccgagtggagtaacactacaatcttcaggagaaagaaaccggtttgcaaacaggatcaaagctctaaaccggttgaaggcgaagctacttgtgatagcaaaagagcaaaaggtttcggatgtaaataaaatcgacagcaagaacattttggaaccgcgggaagaaaccaggagttatgtctctaagggtcacaagatggtggttgatagaaaaaccggtttagagattctggacctgaaatcggtcttggatggaaacattggaccactccttggagctcatattagcatgagaagatcaattgatgcgatttag

SEQ ID NO: 2 - protein, 409 aa, Arabidopsis thaliana
MAAKIIGGCCSWRRFYRKRTSSRFLIFSVRASSSMDDMDTVYKQLGLFSLKKKIKDVVLKAEMFAPDALELEEEQWIKQEETMRYFDLWDDPAKSDEILLKLADRAKAVDSLKDLKYKAEEAKLIIQLGEMDAIDYSLFEQAYDSSLDVSRSLHHYEMSKLLRDQYDAEGACMIIKSGSPGAKSQDLQIWTEQVVSMYIKWAERLGQNARVAEKCSLLSNKSGVSSATIEFEFEFAYGYLLGERGVHRLIISSTSNEECSATVDIIPLFLRASPDFEVKEGDLIVSYPAKEDHKIAENMVCIHHIPSGVTLQSSGERNRFANRIKALNRLKAKLLVIAKEQKVSDVNKIDSKNILEPREETRSYVSKGHKMVVDRKTGLEILDLKSVLDGNIGPLLGAHISMRRSIDAI

SEQ ID NO: 3 - DNA, 4146 bp, Arabidopsis thaliana
  CDS (1)..(4146)
atggcttcgcttgtgtattctccattcactctatccacttctaaagcagagcatctctcttcgctcactaacagtaccaaacattctttcctccggaagaaacacagatcaaccaaaccagccaaatctttcttcaaggtgaaatctgctgtatctggaaacggcctcttcacacagacgaacccggaggtccgtcgtatagttccgatcaagagagacaacgttccgacggtgaaaatcgtctacgtcgtcctcgaggctcagtaccagtcttctctcagtgaagccgtgcaatctctcaacaagacttcgagattcgcatcctacgaagtggttggatacttggtcgaggagcttagagacaagaacacttacaacaacttctgcgaagaccttaaagacgccaacatcttcattggttctctgatcttcgtcgaggaattggcgattaaagttaaggatgcggtggagaaggagagagacaggatggacgcagttcttgtcttcccttcaatgcctgaggtaatgagactgaacaagcttggatcttttagtatgtctcaattgggtcagtcaaagtctccgtttttccaactcttcaagaggaagaaacaaggctctgctggttttgccgatagtatgttgaagcttgttaggactttgcctaaggttttgaagtacttacctagtgacaaggctcaagatgctcgtctctacatcttgagtttacagttttggcttggaggctctcctgataatcttcagaattttgttaagatgatttctggatcttatgttccggctttgaaaggtgtcaaaatcgagtattcggatccggttttgttcttggatactggaatttggcatccacttgctccaaccatgtacgatgatgtgaaggagtactggaactggtatgacactagaagggacaccaatgactcactcaagaggaaagatgcaacggttgtcggtttagtcttgcagaggagtcacattgtgactggtgatgatagtcactatgtggctgttatcatggagcttgaggctagaggtgctaaggtcgttcctatattcgcaggagggttggatttctctggtccagtagagaaatatttcgtagacccggtgtcgaaacagcccatcgtaaactctgctgtctccttgactggttttgctcttgttggtggacctgcaaggcaggatcatcccagggctatcgaagccctgaaaaagctcgatgttccttaccttgtggcagtaccactggtgttccagacgacagaggaatggctaaacagcacacttggtctgcatcccatccaggtggctctgcaggttgccctccctgagcttgatggagcgatggagccaatcgttttcgctggtcgtgaccctagaacagggaagtcacatgctctccacaagagagtggagcaactctgcatcagagcgattcgatggggtgagctcaaaagaaaaactaaggcagagaagaagctggcaatcactgttttcagtttcccacctgataaaggtaatgtagggactgcagcttacctcaatgtgtttgcttccatcttctcggtgttaagagacctcaagagagatggctacaatgttgaaggccttcctgagaatgcagagactcttattgaagaaatcattcatgacaaggaggctcagttcagcagccctaacctcaatgtagcttacaaaatgggagtccgtgagtaccaagacctcactccttatgcaaatgccctggaagaaaactgggggaaacctccggggaaccttaactcagatggagagaaccttcttgtctatggaaaagcgtacggtaatgttttcatcggagtgcaaccaacatttgggtatgaaggtgatcccatgaggctgcttttctccaagtcagcaagtcctcatcacggttttgctgcttactactcttatgtagaaaagatcttcaaagctgatgctgttcttcattttggaacacatggttctctcgagtttatgcccgggaagcaagtgggaatgagtgatgcttgttttcccgacagtcttatcgggaacattcccaatgtctactattatgcagctaacaatccctctgaagctaccattgcaaagaggagaagttatgccaacaccatcagttatttgactcctccagctgagaatgctggtctatacaaagggctgaagcagttgagtgagctgatatcgtcctatcagtctctgaaggacacggggagaggtccacagatcgtcagttccatcatcagcacagctaagcaatgtaatcttgataaggatgtggatcttccagatgaaggcttggagttgtcacctaaagacagagattctgtggttgggaaagtttattccaagattatggagattgaatcaaggcttttgccgtgcgggcttcacgtcattggagagcctccatccgccatggaagctgtggccacactggtcaacattgctgctctagatcgtccggaggatgagatttcagctcttccttctatattagctgagtgtgttggaagggagatagaggatgtttacagaggaagcgacaagggtatcttgagcgatgtagagcttctcaaagagatcactgatgcctcacgtggcgctgtttccgcctttgtggaaaaaacaacaaatagcaaaggacaggtggtggatgtgtctgacaagcttacctcgcttcttgggtttggaatcaatgagccatgggttgagtatttgtccaacaccaagttctacagggcgaacagagataagctcagaacagtgtttggtttccttggagagtgcctgaagttggtggtcatggacaacgaactagggagtctaatgcaagctttggaaggcaagtacgtcgagcctggccccggaggtgatcccatcagaaacccaaaggtcttaccaaccggtaaaaacatccatgccttagatcctcaggctattcccacaacagcagcaatggcaagtgccaagattgtggttgagaggttggtagagagacagaagctcgaaaacgaagggaaatatcccgagacaatcgcgcttgttctttggggaactgacaacatcaaaacatatggggagtctcttgggcaggttctttggatgattggtgtgagaccaattgctgatacttttggaagagtgaaccgtgtcgagcctgtgagcttagaagaactaggaaggccgaggatcgatgtagttgttaactgctcaggggtcttccgtgatctctttatcaaccagatgaaccttcttgaccgagctatcaagatggtggcggagctagatgagcctgtagagcaaaattttgtaaggaaacacgcgttggaacaagcagaggcgcttggcattgatattagagaggcagcgacaagagttttctcaaacgcttcagggtcatactcagccaacatcagtcttgctgttgaaaactcgtcatggaacgatgagaaacagcttcaggacatgtacttgagccgcaaatcgtttgcgtttgatagtgatgctcctggagcaggaatggctgagaagaagcaggtctttgagatggctcttagcactgcagaagtcaccttccagaacctggattcttcagagatttctttgactgatgtgagccactacttcgattctgaccctacaaatctagttcagagtttgaggaaggataagaagaaaccaagctcttacattgctgacactacaactgcaaacgcgcaggtgaggacactatctgagacagtgaggctggacgcaagaacaaagctgctgaatccaaagtggtacgaaggaatgatgtcaagtggatatgaaggagttcgtgagatagagaagagactgtccaacactgtgggatggagtgcaacgtcaggtcaagtagacaattgggtctacgaggaggccaactcaactttcatccaagacgaggagatgctgaaccgtctcatgaacaccaatcccaactccttcaggaaaatgcttcagactttcttggaggccaatggtcgtggctactgggacacttccgctgaaaacatagagaagctcaaggaattgtactcgcaggtggaagacaagatcgaagggatcgatcgataa

SEQ ID NO: 4 - protein, 1381 aa, Arabidopsis thaliana
MASLVYSPFTLSTSKAEHLSSLTNSTKHSFLRKKHRSTKPAKSFFKVKSAVSGNGLFTQTNPEVRRIVPIKRDNVPTVKIVYVVLEAQYQSSLSEAVQSLNKTSRFASYEVVGYLVEELRDKNTYNNFCEDLKDANIFIGSLIFVEELAIKVKDAVEKERDRMDAVLVFPSMPEVMRLNKLGSFSMSQLGQSKSPFFQLFKRKKQGSAGFADSMLKLVRTLPKVLKYLPSDKAQDARLYILSLQFWLGGSPDNLQNFVKMISGSYVPALKGVKIEYSDPVLFLDTGIWHPLAPTMYDDVKEYWNWYDTRRDTNDSLKRKDATVVGLVLQRSHIVTGDDSHYVAVIMELEARGAKVVPIFAGGLDFSGPVEKYFVDPVSKQPIVNSAVSLTGFALVGGPARQDHPRAIEALKKLDVPYLVAVPLVFQTTEEWLNSTLGLHPIQVALQVALPELDGAMEPIVFAGRDPRTGKSHALHKRVEQLCIRAIRWGELKRKTKAEKKLAITVFSFPPDKGNVGTAAYLNVFASIFSVLRDLKRDGYNVEGLPENAETLIEEIIHDKEAQFSSPNLNVAYKMGVREYQDLTPYANALEENWGKPPGNLNSDGENLLVYGKAYGNVFIGVQPTFGYEGDPMRLLFSKSASPHHGFAAYYSYVEKIFKADAVLHFGTHGSLEFMPGKQVGMSDACFPDSLIGNIPNVYYYAANNPSEATIAKRRSYANTISYLTPPAENAGLYKGLKQLSELISSYQSLKDTGRGPQIVSSIISTAKQCNLDKDVDLPDEGLELSPKDRDSVVGKVYSKIMEIESRLLPCGLHVIGEPPSAMEAVATLVNIAALDRPEDEISALPSILAECVGREIEDVYRGSDKGILSDVELLKEITDASRGAVSAFVEKTTNSKGQVVDVSDKLTSLLGFGINEPWVEYLSNTKFYRANRDKLRTVFGFLGECLKLVVMDNELGSLMQALEGKYVEPGPGGDPIRNPKVLPTGKNIHALDPQAIPTTAAMASAKIVVERLVERQKLENEGKYPETIALVLWGTDNIKTYGESLGQVLWMIGVRPIADTFGRVNRVEPVSLEELGRPRIDVVVNCSGVFRDLFINQMNLLDRAIKMVAELDEPVEQNFVRKHALEQAEALGIDIREAATRVFSNASGSYSANISLAVENSSWNDEKQLQDMYLSRKSFAFDSDAPGAGMAEKKQVFEMALSTAEVTFQNLDSSEISLTDVSHYFDSDPTNLVQSLRKDKKKPSSYIADTTTANAQVRTLSETVRLDARTKLLNPKWYEGMMSSGYEGVREIEKRLSNTVGWSATSGQVDNWVYEEANSTFIQDEEMLNRLMNTNPNSFRKMLQTFLEANGRGYWDTSAENIEKLKELYSQVEDKIEGIDR

SEQ ID NO: 5 - DNA, 1929 bp, Arabidopsis thaliana
  CDS (1)..(1929)
atgttcattttcccaaaagacgaaaacagaagagaaactttaacgacaaagctccgtttctccgccgatcatctgacttttaccaccgtgacagaaaaattgagagcaacggcttggagatttgctttctcatccagagctaagtccgtggtagcaatggcagctaatgaagaatttacgggaaatctgaaacgtcaactcgcgaagctctttgatgtttctctaaaattaacggttcctgatgaacctagtgttgagcccttggtggctgcctccgctcttggaaaatttggagattaccaatgtaacaacgcaatgggactatggtccataattaaaggaaagggtactcagttcaagggtcctccagctgttggacaggcccttgttaagagtctccctacttctgagatggtagaatcatgctctgtagctggacctggctttattaatgttgtactatcagctaagtggatggctaagagtattgaaaatatgctcatcgatggagttgacacatgggcacctactctttcggttaagagagctgtagttgatttttcctctcccaacattgcaaaagaaatgcatgttggtcatctaagatcaactatcattggtgacactctagctcgcatgctcgagtactcacatgttgaagttctacgcagaaaccatgttggtgactggggaacacagtttggcatgctaattgagtacctctttgagaaatttcctgatacagatagtgtgaccgagacagcaattggagatcttcaggtgttttacaaggcatcaaaacataaatttgatctggacgaggcctttaaggaaaaagcacaacaggctgtggtccgtctacagggtggtgatcctgtttaccgtaaggcttgggctaagatctgtgacatcagccgaactgagtttgccaaggtttaccaacgccttcgagttgagcttgaagaaaagggagaaagcttttacaaccctcatattgctaaagtaattgaggaattgaatagcaaggggttggttgaagaaagtgaaggtgctcgtgtgattttccttgaaggcttcgacatcccactcatggttgtaaagagtgatggtggttttaactatgcctcaacagatctgactgctctttggtaccggctcaatgaagagaaagctgagtggatcatatatgtgaccgatgttggccagcagcagcactttaatatgttcttcaaagctgccagaaaagcaggttggcttccagacaatgataaaacttaccctagagttaaccatgttggttttggtctcgtccttggggaagatggcaagcgatttagaactcgggcaacagatgtagtccgcctagttgatttgctagatgaggccaagactcgcagtaaacttgcccttattgagcgcggtaaggacaaagaatggacaccggaagaactggaccaaacagctgaggcagttggatatggtgcggtcaagtatgctgacctgaagaacaacagattaacaaattatactttcagctttgatcaaatgcttaatgacaagggaaatacagccgtttaccttctttacgcccatgctcggatctgttcaatcatcagaaagtctggcaaagacatagatgagctgaaaaagacaggaaaattagcattggatcatgcagatgaacgagcactggggcttcacttgcttcgatttgctgagacggtggaggaagcttgtaccaacttattaccgagtgttctgtgcgagtacctctacaatttatctgaacactttaccagattctactccaattgtcaggtcaatggttcaccagaggagacaagccgtctcctactttgtgaagcaacggccatagtcatgcggaaatgcttccaccttcttggaatcactccggtttacaagatttga

SEQ ID NO: 6 - protein, 642 aa, Arabidopsis thaliana
MFIFPKDENRRETLTTKLRFSADHLTFTTVTEKLRATAWRFAFSSRAKSVVAMAANEEFTGNLKRQLAKLFDVSLKLTVPDEPSVEPLVAASALGKFGDYQCNNAMGLWSIIKGKGTQFKGPPAVGQALVKSLPTSEMVESCSVAGPGFINVVLSAKWMAKSIENMLIDGVDTWAPTLSVKRAVVDFSSPNIAKEMHVGHLRSTIIGDTLARMLEYSHVEVLRRNHVGDWGTQFGMLIEYLFEKFPDTDSVTETAIGDLQVFYKASKHKFDLDEAFKEKAQQAVVRLQGGDPVYRKAWAKICDISRTEFAKVYQRLRVELEEKGESFYNPHIAKVIEELNSKGLVEESEGARVIFLEGFDIPLMVVKSDGGFNYASTDLTALWYRLNEEKAEWIIYVTDVGQQQHFNMFFKAARKAGWLPDNDKTYPRVNHVGFGLVLGEDGKRFRTRATDVVRLVDLLDEAKTRSKLALIERGKDKEWTPEELDQTAEAVGYGAVKYADLKNNRLTNYTFSFDQMLNDKGNTAVYLLYAHARICSIIRKSGKDIDELKKTGKLALDHADERALGLHLLRFAETVEEACTNLLPSVLCEYLYNLSEHFTRFYSNCQVNGSPEETSRLLLCEATAIVMRKCFHLLGITPVYKI

SEQ ID NO: 7 - DNA, 1491 bp, Arabidopsis thaliana
  CDS (1)..(1491)
atggtaggagcttcaagaacaatcctatccctatctctatcatcttccctcttcaccttctccaaaatccctcacgtttttccatttctccgcctccacaaacccagattccaccacgcgtttcgtcctctttactccgccgccgcaacaacttcttctccgacgacggagactaatgttacagatccggatcaattgaaacatacgatcttactagagaggcttaggcttcgacatttgaaagaatcagcgaaaccaccacaacagagaccaagtagtgttgttggtgtagaggaagagagtagtattaggaagaagagtaagaagttagttgagaattttcaggaattgggtttaagtgaagaagttatgggagctttacaagagttgaatattgaggttcctactgagattcagtgtatcggaatacctgcggttatggaacgtaagagcgttgtattgggttcgcataccggttctggcaagactcttgcttacttgttgcctattgttcaggtgcttagtgagctgatgagagaagatgaagcaaaccttggtaaaaaaacaaagcctagacgtcccaggactgttgttctttgtcctacaagagaactatctgagcaggtttgtcttcaccaagattatcatcacgcgaggtttagatctatattggttagtggtggttctcggataagaccccaggaggattctttgaacaatgcaatagatatggttgttggaacccctggtaggattcttcagcatatcgaagaaggaaacatggtgtatggagatatcgcatatttggtattggatgaggcagatactatgtttgatcgtggctttggtcccgaaattcgtaaattccttgccccactgaatcaacatattaaggtagtgaatgaaattgtgagttttcaggctgttcagaagttagtcgatgaggagtttcaagggatagagcatttgcgtacatcaacactgcataaaaagatagcaaacgctcgccatgacttcatcaagctttcaggtggtgaagataagctagaagcacttctacaggttcttgaacctagcctagccaaagggagcaaggtgatggtcttctgtaacactttgaactccagtcgcgctgttgatcactatctttctgaaaaccagatctccactgtaaattatcacggtgaagttccagcagaacaaagggttgagaatttgaaaaagttcaaggacgaagaaggagactgtcccacgctagtgtgcacggatttggctgcaaggggtctggacctcgacgttgatcatgtagtcatgtttgatttcccaaagaactcgattgactaccttcatcgcactggaagaacagctcggatgggtgctaaaggtttgtttcatacctctagattatcacttgttaagttctcgtatttcagatggtttcggctagggtggcgtaccaagttttcagatttttttgtttatggactatag

SEQ ID NO: 8 - protein, 496 aa, Arabidopsis thaliana
MVGASRTILSLSLSSSLFTFSKIPHVFPFLRLHKPRFHHAFRPLYSAAATTSSPTTETNVTDPDQLKHTILLERLRLRHLKESAKPPQQRPSSVVGVEEESSIRKKSKKLVENFQELGLSEEVMGALQELNIEVPTEIQCIGIPAVMERKSVVLGSHTGSGKTLAYLLPIVQVLSELMREDEANLGKKTKPRRPRTVVLCPTRELSEQVCLHQDYHHARFRSILVSGGSRIRPQEDSLNNAIDMVVGTPGRILQHIEEGNMVYGDIAYLVLDEADTMFDRGFGPEIRKFLAPLNQHIKVVNEIVSFQAVQKLVDEEFQGIEHLRTSTLHKKIANARHDFIKLSGGEDKLEALLQVLEPSLAKGSKVMVFCNTLNSSRAVDHYLSENQISTVNYHGEVPAEQRVENLKKFKDEEGDCPTLVCTDLAARGLDLDVDHVVMFDFPKNSIDYLHRTGRTARMGAKGLFHTSRLSLVKFSYFRWFRLGWRTKFSDFFVYGL

SEQ ID NO: 9 - DNA, 819 bp, Arabidopsis thaliana
  CDS (1)..(819)
atggcagccatagatatgttcaatagcaacacagatccttttcaagaagagctcatgaaagcacttcaaccttataccaccaacactgattcttcttctcctacgtattcaaacacagtcttcggtttcaatcaaaccacatctctcggtctaaaccagctcacaccttaccaaatccaccaaatccaaaaccagcttaaccagagacgtaacataatctctccaaatctagccccaaagcctgtcccaatgaagaacatgaccgctcagaaactctatagaggagttagacaaaggcactggggaaaatgggtagctgagatccgtttacccaagaaccggacccgactctggcttggaactttcgacacagctgaagaagcagccatggcttatgacctagctgcttacaagctaagaggcgagttcgcgagacttaatttcccacagttcagacacgaggatggatactacggaggaggtagctgtttcaatcctcttcattcctctgtcgacgcaaagctccaagagatttgtcagagcttgagaaaaacagaggatattgacctcccctgttctgaaacagagcttttcccgccaaaaacagagtatcaagaaagtgaatatgggttcttgagatctgatgagaattcgttttcagatgagtctcatgtggaatcttcttcgccggaatctggtattactacgttcttggacttttcggattctggatttgatgagattgggagtttcgggctggagaagtttccttctgtggagattgattgggatgcgattagcaaattgtccgaatcttaa

SEQ ID NO: 10 - protein, 272 aa, Arabidopsis thaliana
MAAIDMFNSNTDPFQEELMKALQPYTTNTDSSSPTYSNTVFGFNQTTSLGLNQLTPYQIHQIQNQLNQRRNIISPNLAPKPVPMKNMTAQKLYRGVRQRHWGKWVAEIRLPKNRTRLWLGTFDTAEEAAMAYDLAAYKLRGEFARLNFPQFRHEDGYYGGGSCFNPLHSSVDAKLQEICQSLRKTEDIDLPCSETELFPPKTEYQESEYGFLRSDENSFSDESHVESSSPESGITTFLDFSDSGFDEIGSFGLEKFPSVEIDWDAISKLSES

SEQ ID NO: 11 - DNA, 1476 bp, Arabidopsis thaliana
  CDS (1)..(1476)
atgtggaaggccaagacatgcttccgtcagatttacttgaccgtactaatacggcggtactcgagagtcgctccgccgccgtcttcggtgatccgcgtgacaaacaacgtagcacacctgggaccaccgaagcaaggaccactgccacgtcagctgatatccctgccgccatttcccggtcatccattacctggcaaaaacgccggagctgacggcgacgatggagatagcggcggccacgtcacagctataagctgggtcaagtactattttgaagaaatctatgataaggctattcaaactcatttcacaaagggccttgttcagatggagtttcgaggtcgtagggatgcttcaagagagaaagaagatggagctattcctatgagaaagattaagcataacgaggtgatgcaaataggagacaaaatctggttgccggtttcaatcgctgagatgaggatttctaagagatatgacaccataccaagtggaaccttgtatccaaacgcagacgaaatcgcatatcttcaaaggcttgtcaggttcaaggactctgctattatagttcttaataagccacctaagcttccagtcaagggaaatgtgcctatacataatagcatggatgcacttgcagctgcagctttgtcttttggtaacgatgaaggtcctagattggtaaaactcacttttttgggggtacatcgtcttgatagggaaactagtggcctcttagtaatgggtcgaaccaaagaaagtatagattatcttcactcagtgttcagtgactacaaggggagaaactcaagctgtaaggcttggaacaaagcgtgtgaggcgatgtatcagcaatattgggcattggtgattggttctccaaaggaaaaagaaggactaatttcagctcctctttcaaaggtgcttttggacgatggtaaaacagacagggtggttttggctcaaggttcgggctttgaagcttcgcaagatgcaataacagagtataaagtgttaggacctaagatcaacgggtgttcgtgggtagaacttcgtcctattactagcagaaaacatcagccaccttctaaaaaacagctacgtgtacactgcgctgaagcacttggtactccaatagtaggggattacaagtacggttggtttgttcacaagagatggaaacagatgcctcaggttgatatcgaaccaactactgggaaaccatataaactgcgcagaccagaaggtcttgatgtccaaaagggaagcgttttgtcaaaagtacctttgttacatctccattgccgggaaatggtacttccaaacattgccaagttcctacatgtcatgaaccaacaggaaacagagccgcttcacacaggaatcattgataaaccggatctcttgcggtttgtagcttcaatgcccagccatatgaagatcagttggaacttaatgtcttcatatttggtgtag

SEQ ID NO: 12 - protein, 491 aa, Arabidopsis thaliana
MWKAKTCFRQIYLTVLIRRYSRVAPPPSSVIRVTNNVAHLGPPKQGPLPRQLISLPPFPGHPLPGKNAGADGDDGDSGGHVTAISWVKYYFEEIYDKAIQTHFTKGLVQMEFRGRRDASREKEDGAIPMRKIKHNEVMQIGDKIWLPVSIAEMRISKRYDTIPSGTLYPNADEIAYLQRLVRFKDSAIIVLNKPPKLPVKGNVPIHNSMDALAAAALSFGNDEGPRLVKLTFLGVHRLDRETSGLLVMGRTKESIDYLHSVFSDYKGRNSSCKAWNKACEAMYQQYWALVIGSPKEKEGLISAPLSKVLLDDGKTDRVVLAQGSGFEASQDAITEYKVLGPKINGCSWVELRPITSRKHQPPSKKQLRVHCAEALGTPIVGDYKYGWFVHKRWKQMPQVDIEPTTGKPYKLRRPEGLDVQKGSVLSKVPLLHLHCREMVLPNIAKFLHVMNQQETEPLHTGIIDKPDLLRFVASMPSHMKISWNLMSSYLV

SEQ ID NO: 13 - DNA, 855 bp, Arabidopsis thaliana
  CDS (1)..(855)
atggcgagattagtgcgtgtggctagatcctcctccctctttggctttggtaaccgtttctactctacttcagccgaagctagccacgcgtcgtcgccttcgccgtttcttcacggcggcggagctagcagggttgctccgaaagatagaaatgttcagtgggtgtttttgggatgtcctggtgttggaaaaggaacttacgctagtagactatcaacccttctcggcgttcctcacatcgccaccggcgatctcgtccgtgaagagcttgcatcttctggacctctctctcaaaagctatcggagattgtaaatcagggaaaattggtttctgatgagatcattgtagacttattgtccaaaagacttgaggctggtgaagctagaggtgaatcagggtttatccttgatggctttcctcgtaccatgagacaagctgaaatactgggagatgtaactgacatcgatttggtggtgaatttgaagcttcctgaggaagttttggttgacaaatgccttggaaggagaacatgtagtcaatgtggcaagggttttaatgtagctcacatcaacttaaagggtgagaatggaagacctggaattagtatggatccacttctccctccacatcaatgtatgtcaaagcttgtcactcgagctgatgatactgaagaggtggtgaaagcaaggcttcgtatatacaatgaaacgagccagcctcttgaagaatactaccgtaccaagggaaagcttatggagtttgacttacctggaggcatcccagagtcatggccaaggctattggaagctttaaggcttgacgattacgaggagaaacagtctgtcgcagcataa

SEQ ID NO: 14 - protein, 284 aa, Arabidopsis thaliana
MARLVRVARSSSLFGFGNRFYSTSAEASHASSPSPFLHGGGASRVAPKDRNVQWVFLGCPGVGKGTYASRLSTLLGVPHIATGDLVREELASSGPLSQKLSEIVNQGKLVSDEIIVDLLSKRLEAGEARGESGFILDGFPRTMRQAEILGDVTDIDLVVNLKLPEEVLVDKCLGRRTCSQCGKGFNVAHINLKGENGRPGISMDPLLPPHQCMSKLVTRADDTEEVVKARLRIYNETSQPLEEYYRTKGKLMEFDLPGGIPESWPRLLEALRLDDYEEKQSVAA

SEQ ID NO: 15 - DNA, 1491 bp, Arabidopsis thaliana
  CDS (1)..(1491)
atgcagatttgccaaaccaagctcaatttcactttccctaatcccacaaaccctaatttctgcaaacccaaagctcttcaatggtcaccgcctcgtcgcatatccttgctgccttgtcgtggattcagctccgatgaattcccagtcgacgaaaccttcctcgagaaattcggaccaaaggacaaagacacagaagatgaagctcgacgacgtaactggatcgaacgtggttgggctccatgggaagagattctcacaccagaagctgatttcgctcgtaaatctctcaacgaaggtgaagaagttccgcttcaatcgccggaagcgatcgaagcgtttaagatgctgagaccatcgtataggaagaagaagattaaggagatggggataacagaagacgaatggtatgcaaagcaatttgagattagaggtgataaaccacctcctttagaaacatcttgggctggtccgatggttcttaggcaaattccgccgcgtgattggcctcccagaggttgggaagttgataggaaggagctggagtttattagggaagctcataagttaatggctgaaagagtttggcttgaggatttggataaggatttgagagttggtgaagatgctactgttgataagatgtgtttggagaggtttaaggttttcttgaaacaatacaaggaatgggttgaagataataaagataggttggaggaagaatcttacaagctcgatcaggatttttatccgggtaggaggaaaagagggaaggattacgaagatgggatgtatgagcttcccttttactatccagggatggcacagttaccactttacatctgtatcagggagcgtttgttgacattggaggtgttcatgaagggtatgtttatgtctctttactttgtaaagatagacttaccgtggttcttgtatttaggatgggtacctataaaaggtaatgactggttttggatccggcatttcataaaagttgggatgcatgttatcgttgaaatcacggcaaaaagagatccataccggtttcggtttcccttggagttgcgcttcgtccatcctaacatagatcacatgatatttaataaatttgacttcccaccaatattccatcgtgatggggatactaatccagatgagatacggcgagattgtggaagacctcctgaacctagaaaagatccaggatcaaagccagaggaggaagggctgctctctgatcacccttatgtcgacaagttgtggcagatacatgtagctgagcaaatgattttgggtgattacgaagctaaccctgcaaaatacgaaggcaaaaagctatcagaattatctgatgatgaagactttgatgaacaaaaggatatcgagtatggcgaagcttattataagaaaaccaaattgccaaaagtgattctgaaaaccagtgtcaaggaacttgacttagaggctgcattgaccgagcgccaggtttaa

SEQ ID NO: 16 - protein, 496 aa, Arabidopsis thaliana
MQICQTKLNFTFPNPTNPNFCKPKALQWSPPRRISLLPCRGFSSDEFPVDETFLEKFGPKDKDTEDEARRRNWIERGWAPWEEILTPEADFARKSLNEGEEVPLQSPEAIEAFKMLRPSYRKKKIKEMGITEDEWYAKQFEIRGDKPPPLETSWAGPMVLRQIPPRDWPPRGWEVDRKELEFIREAHKLMAERVWLEDLDKDLRVGEDATVDKMCLERFKVFLKQYKEWVEDNKDRLEEESYKLDQDFYPGRRKRGKDYEDGMYELPFYYPGMAQLPLYICIRERLLTLEVFMKGMFMSLYFVKIDLPWFLYLGWVPIKGNDWFWIRHFIKVGMHVIVEITAKRDPYRFRFPLELRFVHPNIDHMIFNKFDFPPIFHRDGDTNPDEIRRDCGRPPEPRKDPGSKPEEEGLLSDHPYVDKLWQIHVAEQMILGDYEANPAKYEGKKLSELSDDEDFDEQKDIEYGEAYYKKTKLPKVILKTSVKELDLEAALTERQV

SEQ ID NO: 17 - DNA, 1095 bp, Arabidopsis thaliana
  CDS (1)..(1095)
atgttacagtccattcatcttcgtttttcctccacaccatcaccttctaaaagagaatctctcataattccatcggttatttgctcatttcctttcacctcttcttcgttccgtccaaagcaaacccagaaactgaagcgtctggttcaattttgcgctccttacgaggtcggaggtggatacaccgatgaagaattgttcgaaagatacggaactcagcaaaatcaaactaatgtcaaagataaattagatccagctgagtatgaagctttgcttaaaggaggcgaacaagtgacttccgttcttgaagaaatgattaccctcttggaagatatgaagatgaatgaagcatctgagaatgttgctgtagaattggctgcacaaggagttatagggaaaagggttgatgaaatggaatcagggtttatgatggctcttgattacatgatccaacttgcagacaaagaccaagacgagaaggtccaggtgattggtttactctgtagaaccccgaaaaaggaaagtagacatgagcttctgcgtagggtggctgcaggtggtggggcttttgaaagtgagaacggtactaaacttcatatacccggagcaaatctgaatgacatagctaatcaagctgatgacttgctagagactatggaaacaaggccagctattccggatcgaaaactactagcgaggcttgttttgattagagaggaagcccggaacatgatgggaggaggtatacttgatgaaagaaatgaccgaggtttcactactcttcctgaatcagaggtgaatttcttagccaaattggtagctttgaaacctggaaagactgtgcagcagatgatccagaatgtaatgcaagggaaagatgaaggcgcagataatcttagcaaagaagacgattcttctaccgaaggaagaaaaccaagtggattaaatggaaggggaagcgttacaggaagaaaaccgttaccagtaagaccaggaatgtttctagaaactgtcacaaaggtactgggaagtatatactcgggtaatgcctccgggataacagcacaacatctagaatgggtaagttcctcataa

SEQ ID NO: 18 - protein, 364 aa, Arabidopsis thaliana
MLQSIHLRFSSTPSPSKRESLIIPSVICSFPFTSSSFRPKQTQKLKRLVQFCAPYEVGGGYTDEELFERYGTQQNQTNVKDKLDPAEYEALLKGGEQVTSVLEEMITLLEDMKMNEASENVAVELAAQGVIGKRVDEMESGFMMALDYMIQLADKDQDEKVQVIGLLCRTPKKESRHELLRRVAAGGGAFESENGTKLHIPGANLNDIANQADDLLETMETRPAIPDRKLLARLVLIREEARNMMGGGILDERNDRGFTTLPESEVNFLAKLVALKPGKTVQQMIQNVMQGKDEGADNLSKEDDSSTEGRKPSGLNGRGSVTGRKPLPVRPGMFLETVTKVLGSIYSGNASGITAQHLEWVSSS

SEQ ID NO: 19 - DNA, 465 bp, Arabidopsis thaliana
  CDS (1)..(465)
atggctatggcggcgtctattatccaatcttctccgctctccttcaatagcaacaacgcaaagccacggattcatagttcaggatcgctcggcggaatcaaaagccaaaatagagtctctccattgagtgcggttggattaagctcaggccttggaagtagaaggaaatctcttttgatatgtcactcagccattaacgcgaaatgcagtgaaggacaaacacagaccgttactcgggagtcaccgactataacacaggctcctgtacactctaaggagaaatcaccaagcctagacgatggaggagacgggttcccaccgcgagatgatggagatggtggtggaggaggagggggtggaggcaactggtcgggtgggttcttcttctttggttttctggccttcttgggtctattgaaggataaagagggcgaggaagattaccgagggagcagaaggcgataa

SEQ ID NO: 20 - protein, 154 aa, Arabidopsis thaliana
MAMAASIIQSSPLSFNSNNAKPRIHSSGSLGGIKSQNRVSPLSAVGLSSGLGSRRKSLLICHSAINAKCSEGQTQTVTRESPTITQAPVHSKEKSPSLDDGGDGFPPRDDGDGGGGGGGGGNWSGGFFFFGFLAFLGLLKDKEGEEDYRGSRRR

SEQ ID NO: 21 - DNA, 642 bp, Arabidopsis thaliana
  CDS (1)..(642)
atgacgacagtgaccaccagcttcgtctctttctcgccggcattgatgatttttcagaagaaatcacgacgatcctctccaaatttccgcaatcgatccacgtctcttcccatagtttcagcaacattaagccacatagaagaagcagccacaacaacaaatctcattcgacagacgaattccatttcggaatcgttgcgtaacatttctctagcagatttagatccaggaacagcgaagctcgctattggtatcttaggtccagctttatcagcttttggatttctattcattttgagaatcgttatgtcttggtacccgaaacttcccgttgacaagtttccgtacgttttagcttacgctccgacagaaccaatccttgttcagacaaggaaagtgattccaccacttgcaggtgttgatgttactcctgtggtttggtttgggcttgtagttgcggctgcggcagacgcatatgaaattgttcgttttgttgccgccagtacttgcgcggcgacgaaacgaacatatgcacctgcggcaatggcagcggtagagtttgctaccgccgctgccgcctgcggtgatgaaacgaacagactaattataatcgagtcgagattcttcaaagctatatattga

SEQ ID NO: 22 - protein, 213 aa, Arabidopsis thaliana
MTTVTTSFVSFSPALMIFQKKSRRSSPNFRNRSTSLPIVSATLSHIEEAATTTNLIRQTNSISESLRNISLADLDPGTAKLAIGILGPALSAFGFLFILRIVMSWYPKLPVDKFPYVLAYAPTEPILVQTRKVIPPLAGVDVTPVVWFGLVVAAAADAYEIVRFVAASTCAATKRTYAPAAMAAVEFATAAAACGDETNRLIIIESRFFKAIY

SEQ ID NO: 23 - DNA, 3066 bp, Arabidopsis thaliana
  CDS (1)..(3066)
atggtgtctccactctgcgactctcagttactttaccaccgcccctcgatctcacctaccgcttctcagttcgtgatcgcggatggaatcatcctccggcaaaatcgtcttctgagctcttcgtcgttttggggcaccaaattcggaaacaccgtcaagttgggagtatctggatgtagtagctgctctcggaagagaagcacgagtgtgaatgcttcactaggtggtcttcttagcggaattttcaagggttctgataacggagagtcgactaggcaacagtacgcatccatcgtcgcatccgttaatcgcttggagactgagatttcggctctttcggattctgagttgcgagagaggactgatgcgttgaagcaacgtgctcagaaaggagaatccatggattcacttttacctgaagcatttgctgttgtgagagaagcttccaagagagttcttggactcagacctttcgatgtgcaattaattggtggtatggttcttcataaaggagaaatagctgaaatgagaactggtgaagggaaaacgcttgttgctattttaccagcttatttgaatgcattaagtgggaaaggtgttcatgtggttacagttaatgattatcttgctcgaagagattgtgaatgggttggtcaagttcctcggttccttggattgaaggttggtctaatccaacagaatatgacacctgaacaaagaaaggaaaattatttatgcgatatcacatatgtcaccaacagtgagcttggatttgattatctgagagacaatctagccacggaaagtgttgaggagctcgtcttgagggatttcaattattgtgtgattgatgaagttgattccatacttattgatgaagcaaggactcctctcattatctctgggcctgcagagaaacctagtgaccaatattacaaagctgcaaagattgcttcagcctttgagcgggatatacattacactgttgatgaaaagcagaagactgttttactgacggaacagggttatgaggatgcagaagaaatcctggacgtgaaagatttgtatgatccccgtgaacagtgggcatcatatgttcttaatgccattaaggcaaaagaactttttctcagagatgtgaactatatcatccgagcaaaggaggttcttatcgtggatgagtttactggtcgtgtaatgcagggaagacgttggagtgatggactacatcaagctgttgaagcaaaagaaggcttgcctattcagaatgaatctattactctggcgtcaattagttatcaaaacttctttctgcagtttccgaaactttgcgggatgacgggtacagcatcgaccgagagtgcagaatttgaaagcatatacaagcttaaagttacaattgtacccacaaataagcccatgataagaaaggatgagtcagatgtggttttcaaggcagtcaatggcaaatggcgggcagtagtagtggagatctctagaatgcacaagacaggtagggctgtgctagttggcacaaccagtgtcgagcagagtgatgaactatcgcaactgttgagggaagctggaataactcatgaggtcctcaatgccaagccagaaaatgtggagagggaagctgaaattgtagcacaaagtggccgtttaggggcagtaacaattgccacaaatatggcagggcgtgggacagacataattcttggtggaaacgcagagttcatggcacgtttgaagcttcgtgagatacttatgcccagagtggtaaagcctactgatggtgtttttgtatctgtgaagaaggcccctcccaagagaacatggaaggtgaatgagaagttatttccatgcaaactgtcaaatgagaaagcaaagctagctgaagaagctgtacaatcagctgtagaggcttggggccagaaatcgttaactgagcttgaagcagaggaacgtttatcttattcttgtgaaaagggtcctgtccaagatgaagttataggtaaactgaggactgcatttctggcgatagcgaaagaatataagggctacactgatgaagaaaggaagaaggttactggtggacttcacgtggtggggacagagcggcatgaatcacgtcgaatagacaatcagttgcgtgggcgaagtggccggcaaggggatcctggaagttcccgattcttccttagtcttgaagataacatattccgcatttttggtggagatcggattcagggtatgatgagggcattcagggtggaagatttaccgatcgaatccaagatgcttactaaagctctagatgaagctcagagaaaagttgagaattacttctttgacatcagaaagcaattattcgaatttgacgaggttctcaatagccaaagagatcgtgtttatacagagagaaggcgtgctcttgtgtcggacagccttgagcctctgattatcgagtatgctgaattgacaatggatgacattctagaggcaaatattggcccagatactccaaaggaaagctgggattttgaaaagctcattgcgaaagttcagcagtactgttacctgttgaacgatctcactcccgatttgctgaaaagcgaaggatcaagttatgaagggttgcaagattatctccgtgcccgtggccgcgatgcatacttacagaaaagagaaatcgtggagaaacaatcaccagggctaatgaaagatgccgaacgattcttaatcttgagcaatattgataggttatggaaagaacaccttcaagcactcaagttcgtgcaacaagctgtggggctcagaggatatgcgcaacgcgatccactcatcgagtataagctcgaaggatacaatctatttctggaaatgatggctcaaatacgaagaaatgtgatatactccatatatcagtttcaaccagtgcgggtaaagaaggacgaagagaagaagtctcagaacgggaaaccgagcaaacaagtagataatgctagtgagaagcctaaacaagttggtgtcacagatgagccatcctcaattgcaagcgcctaa

SEQ ID NO: 24 - protein, 1021 aa, Arabidopsis thaliana
MVSPLCDSQLLYHRPSISPTASQFVIADGIILRQNRLLSSSSFWGTKFGNTVKLGVSGCSSCSRKRSTSVNASLGGLLSGIFKGSDNGESTRQQYASIVASVNRLETEISALSDSELRERTDALKQRAQKGESMDSLLPEAFAVVREASKRVLGLRPFDVQLIGGMVLHKGEIAEMRTGEGKTLVAILPAYLNALSGKGVHVVTVNDYLARRDCEWVGQVPRFLGLKVGLIQQNMTPEQRKENYLCDITYVTNSELGFDYLRDNLATESVEELVLRDFNYCVIDEVDSILIDEARTPLIISGPAEKPSDQYYKAAKIASAFERDIHYTVDEKQKTVLLTEQGYEDAEEILDVKDLYDPREQWASYVLNAIKAKELFLRDVNYIIRAKEVLIVDEFTGRVMQGRRWSDGLHQAVEAKEGLPIQNESITLASISYQNFFLQFPKLCGMTGTASTESAEFESIYKLKVTIVPTNKPMIRKDESDVVFKAVNGKWRAVVVEISRMHKTGRAVLVGTTSVEQSDELSQLLREAGITHEVLNAKPENVEREAEIVAQSGRLGAVTIATNMAGRGTDIILGGNAEFMARLKLREILMPRVVKPTDGVFVSVKKAPPKRTWKVNEKLFPCKLSNEKAKLAEEAVQSAVEAWGQKSLTELEAEERLSYSCEKGPVQDEVIGKLRTAFLAIAKEYKGYTDEERKKVTGGLHVVGTERHESRRIDNQLRGRSGRQGDPGSSRFFLSLEDNIFRIFGGDRIQGMMRAFRVEDLPIESKMLTKALDEAQRKVENYFFDIRKQLFEFDEVLNSQRDRVYTERRRALVSDSLEPLIIEYAELTMDDILEANIGPDTPKESWDFEKLIAKVQQYCYLLNDLTPDLLKSEGSSYEGLQDYLRARGRDAYLQKREIVEKQSPGLMKDAERFLILSNIDRLWKEHLQALKFVQQAVGLRGYAQRDPLIEYKLEGYNLFLEMMAQIRRNVIYSIYQFQPVRVKKDEEKKSQNGKPSKQVDNASEKPKQVGVTDEPSSIASA

SEQ ID NO: 25 - DNA, 660 bp, Arabidopsis thaliana
  CDS (1)..(660)
atgagcttggcttcgattccctcgtcgtcaccagtggcttcaccgtacttccgctgccgtacttacatcttctccttctcttcctcacctctctgtttatatttcccgcgcggtgactctacttctctcaggccacgagttcgcgccttgcgaacggaatctgacggtgctaaaatcggtaactcggagtcttacggctccgaattgcttcgtcggcctcgtattgcgtcggaggaaagctccgaagaagaggaggaagaggaagaagagaacagcgaaggtgatgagttcgtcgattgggaagataaaatccttgaggttactgttcctcttgttggcttcgtcagaatgattcttcactccggaaaatatgcaaaccgagataggctaagccccgagcatgagagaacaattattgagatgctacttccttatcatcctgaatgtgagaagaagatcggatgtggtatagactatattatggtagggcatcacccggattttgagagctctcgatgtatgtttatagttcgaaaagatggagaagtagtcgacttttcgtattggaaatgcataaaaggtcttataaaaaagaagtatcctctgtatgcagacagtttcatcctcagacattttcgcaaacgtaggcagaacagatga

SEQ ID NO: 26 - protein, 219 aa, Arabidopsis thaliana
MSLASIPSSSPVASPYFRCRTYIFSFSSSPLCLYFPRGDSTSLRPRVRALRTESDGAKIGNSESYGSELLRRPRIASEESSEEEEEEEEENSEGDEFVDWEDKILEVTVPLVGFVRMILHSGKYANRDRLSPEHERTIIEMLLPYHPECEKKIGCGIDYIMVGHHPDFESSRCMFIVRKDGEVVDFSYWKCIKGLIKKKYPLYADSFILRHFRKRRQNR

SEQ ID NO: 27 - DNA, 1929 bp, Arabidopsis thaliana; identical to SEQ ID NO: 5
  CDS (1)..(1929)

SEQ ID NO: 28 - protein, 642 aa, Arabidopsis thaliana; identical to SEQ ID NO: 6

SEQ ID NO: 29 - DNA, 1698 bp, Arabidopsis thaliana
  CDS (1)..(1698)
atggcttcgaccccgaagcttaccagtacaatttcatcatcttctccatctcttcaattcctctgcaaaaaactcccaatcgcaattcatctaccatcatcttcttcctctagctttctctcgcttcctaaaaccctaacctctctctattctctccgtccccgtatcgccctactctcaaaccaccgctattaccactctcgccggttttctgtttgtgccagtaccgataatggagctgaatcagaccgccactacgattttgatctcttcactatcggtgccggaagcggcggcgtccgcgcctctcgcttcgccactagcttcggtgcatccgccgccgtttgcgagcttcctttttccactatctcttccgatactgctggaggcgttggaggaacgtgtgtattgagaggatgtgtaccaaagaagttacttgtgtatgcatccaaatacagtcatgagtttgaagacagtcatggatttggttggaagtatgagactgagccttctcatgattggactactttgattgctaacaagaatgctgagttacagcggttgactggtatttataagaatatactgagcaaagctaatgtcaagttgattgaaggtcgtggaaaggttatagacccacacactgttgatgtagatgggaaaatctatactacgaggaatattctgattgcagttggtggacgtcctttcattcctgacattccaggaaaagagtttgctattgattctgatgccgcgcttgatttgccttccaagcctaagaaaattgcaatagttggtggtggctacatagccctggagtttgcggggatcttcaatggtcttaactgtgaagttcatgtatttataaggcaaaagaaggtgctgaggggatttgatgaagatgtcagggatttcgttggagagcagatgtctttaagaggtattgagtttcacactgaagaatcccctgaagccatcatcaaagctggagatggctcgttctctctgaagaccagcaagggaactgttgagggattttcgcatgttatgtttgcaactggtcgcaagcccaacacaaagaacttagggttggagaatgttggcgttaaaatggcgaaaaatggagcaatagaggttgacgaatattcacagacatctgttccatccatctgggctgttggggatgttactgaccgaatcaatttgactccagttgctttgatggagggaggtgcattggctaaaactttgtttcaaaatgagccaacaaagcctgattatagagctgttccctgcgccgttttctcccagccacctattggaacagttggtctaactgaagagcaggccatagaacaatatggtgatgtggatgtttacacatcgaactttaggccattaaaggctaccctttcaggacttccagaccgagtatttatgaaactcattgtctgtgcaaacaccaataaagttctcggtgttcacatgtgtggagaagattcaccagaaatcatccagggatttggggttgcagttaaagctggtttaactaaggccgactttgatgctacagtgggtgttcaccccacagcagctgaggagtttgtcactatgagggctccaaccaggaaattccgcaaagactcctctgagggaaaggcaagtcctgaagctaaaacagctgctggggtgtag

SEQ ID NO: 30 - protein, 565 aa, Arabidopsis thaliana
MASTPKLTSTISSSSPSLQFLCKKLPIAIHLPSSSSSSFLSLPKTLTSLYSLRPRIALLSNHRYYHSRRFSVCASTDNGAESDRHYDFDLFTIGAGSGGVRASRFATSFGASAAVCELPFSTISSDTAGGVGGTCVLRGCVPKKLLVYASKYSHEFEDSHGFGWKYETEPSHDWTTLIANKNAELQRLTGIYKNILSKANVKLIEGRGKVIDPHTVDVDGKIYTTRNILIAVGGRPFIPDIPGKEFAIDSDAALDLPSKPKKIAIVGGGYIALEFAGIFNGLNCEVHVFIRQKKVLRGFDEDVRDFVGEQMSLRGIEFHTEESPEAIIKAGDGSFSLKTSKGTVEGFSHVMFATGRKPNTKNLGLENVGVKMAKNGAIEVDEYSQTSVPSIWAVGDVTDRINLTPVALMEGGALAKTLFQNEPTKPDYRAVPCAVFSQPPIGTVGLTEEQAIEQYGDVDVYTSNFRPLKATLSGLPDRVFMKLIVCANTNKVLGVHMCGEDSPEIIQGFGVAVKAGLTKADFDATVGVHPTAAEEFVTMRAPTRKFRKDSSEGKASPEAKTAAGV

SEQ ID NO: 31 - DNA, 1719 bp, Arabidopsis thaliana
  CDS (1)..(1719)
atgtcttcttgtcttcttcctcagttcaagtgcccacctgattctttctctattcacttccgaacctctttctgtgcccctaaacacaacaagggttcagtcttcttccaaccgcaatgtgcagtatccacttcaccggcgttattaacttctatgcttgatgtcgcaaagcttagactaccctctttcgatactgattcggattcccttatatcagacaggcagtggacttatacaaggcccgatggtccttccactgaggcgaagtatttagaagctttagcctctgagacacttctcacaagcgatgaagcagtagttgtagcagcagcagctgaagcagtcgcccttgcaagagctgctgtcaaagttgccaaagatgcaacattatttaagaacagtaacaacacgaacctattaacttcgtcaacggccgacaaacgctccaagtgggaccagtttactgagaaggaacgtgctggcatattggggcatctagcggtttcggacaatggaattgtgagtgataaaatcactgcatctgcctctaacaaagagtctattggtgatttagaatcagaaaaacaagaagaagttgagcttctggaggagcaaccttcagtgagtttagctgtgagatctacacgtcaaactgaaaggaaagctcggagggcaaaagggttagagaaaactgcatcaggtattccgtctgtgaagactggttcgagccctaaaaagaaacgtcttgttgcgcaggaagttgatcataatgatcctttgcgttatctaagaatgacaacaagcagttccaagcttctcactgtcagagaagaacatgagctgtcggcaggaatacaggaccttctgaagttagaaagacttcaaacagagcttacagagcgtagtggacgtcagccaacctttgcgcagtgggcttctgctgctggagtcgatcagaaatcattaaggcaacgtatacatcatggcacactatgcaaagacaaaatgatcaaaagcaacattcgactcgttatttcgattgcaaagaattatcaaggagctgggatgaacctccaagatcttgtccaggaagggtgcagagggcttgtgaggggagcagagaagtttgatgctacaaagggttttaaattttcgacttacgcgcattggtggatcaagcaagctgtgcggaagtctctctctgatcagtccagaatgataagattgccttttcacatggtggaagcaacatatagggtgaaagaggcacgaaagcaactgtacagtgaaaccggtaagcacccaaagaacgaagaaattgcagaggcaacagggctgtcgatgaagagactcatggcggttctactctctcctaaacctccgaggtcgctagaccagaaaatcggaatgaatcaaaacctcaaaccttcggaagtgatagcagatccagaagcagtaacgtcagaagatatactgataaaggaattcatgaggcaggacttggacaaagtgttggactcgttgggtacaagggagaaacaagtgatacgttggagatttgggatggaggatgggagaatgaagacgttgcaagagataggagagatgatgggagtgagcagggagagagtaagacagatagagtcatctgcattcaggaaactaaagaacaagaagagaaacaaccatttgcagcaatacttggttgcacaatcataa

SEQ ID NO: 32 - protein, 572 aa, Arabidopsis thaliana
MSSCLLPQFKCPPDSFSIHFRTSFCAPKHNKGSVFFQPQCAVSTSPALLTSMLDVAKLRLPSFDTDSDSLISDRQWTYTRPDGPSTEAKYLEALASETLLTSDEAVVVAAAAEAVALARAAVKVAKDATLFKNSNNTNLLTSSTADKRSKWDQFTEKERAGILGHLAVSDNGIVSDKITASASNKESIGDLESEKQEEVELLEEQPSVSLAVRSTRQTERKARRAKGLEKTASGIPSVKTGSSPKKKRLVAQEVDHNDPLRYLRMTTSSSKLLTVREEHELSAGIQDLLKLERLQTELTERSGRQPTFAQWASAAGVDQKSLRQRIHHGTLCKDKMIKSNIRLVISIAKNYQGAGMNLQDLVQEGCRGLVRGAEKFDATKGFKFSTYAHWWIKQAVRKSLSDQSRMIRLPFHMVEATYRVKEARKQLYSETGKHPKNEEIAEATGLSMKRLMAVLLSPKPPRSLDQKIGMNQNLKPSEVIADPEAVTSEDILIKEFMRQDLDKVLDSLGTREKQVIRWRFGMEDGRMKTLQEIGEMMGVSRERVRQIESSAFRKLKNKKRNNHLQQYLVAQS

SEQ ID NO: 33 - DNA, 564 bp, Arabidopsis thaliana
  CDS (1)..(564)
atgtcaaacgtgagttttcttgagttgcagtacaagctctccaagaacaagatgttgaggaagccttcaaggatgttctctagagatagacaatcctcagggctatcttcacctggaccaggaggcttctctcagccttctgtgaatgagatgagacgtgttttcagcaggtttgatttggataaagacgggaaaatctctcagactgagtacaaggtggtgctgagagcgctaggacaagagcgggcgatcgaggatgtgcctaagatctttaaggctgtggatctggacggtgatgggtttattgatttcagggagtttattgatgcatacaagagaagtggtgggattaggtcttcggatatacgaaattctttctggacttttgatttgaacggcgatgggaagataagcgcagaggaagtgatgtcggttctgtggaagcttggtgagagatgtagcttagaggactgcaacaggatggttagagctgttgatgcagatggtgatggattggttaatatggaagagttcatcaaaatgatgtcttccaacaatgtctaa

SEQ ID NO: 34 - protein, 187 aa, Arabidopsis thaliana
MSNVSFLELQYKLSKNKMLRKPSRMFSRDRQSSGLSSPGPGGFSQPSVNEMRRVFSRFDLDKDGKISQTEYKVVLRALGQERAIEDVPKIFKAVDLDGDGFIDFREFIDAYKRSGGIRSSDIRNSFWTFDLNGDGKISAEEVMSVLWKLGERCSLEDCNRMVRAVDADGDGLVNMEEFIKMMSSNNV

SEQ ID NO: 35 - DNA, 1809 bp, Arabidopsis thaliana
  CDS (1)..(1809)
atggattcatcatcgacgaaatcgaagatctcacattcacgcaagacgaacaaaaagtcaaacaagaagcacgaatcaaatgggaaacaacaacaacaacaagacgtcgatggtggtggtgggtgtttgagatcatcatggatctgcaagaatgcatcgtgtagagctaatgtgcctaaagaagattccttttgcaagagatgttcttgttgtgtttgtcataatttcgatgaaaacaaggatcctagtctttggttagtttgtgagcctgagaaatctgatgatgttgagttctgtggcttatcgtgtcacattgagtgtgcttttcgagaagtcaaagttggtgttattgctcttgggaatctgatgaagcttgatggttgtttttgttgctactcatgtggcaaagtttctcaaattcttggatgttggaaaaagcagcttgtggcagcaaaggaagcacgacgacgtgatggactgtgttatagaatagatttgggttatagactgttgaatgggactagtcggtttagtgaattgcatgagattgttagagctgctaagtctatgctggaggatgaagttggacctcttgatggacctactgctagaactgatagaggcattgttagtaggcttcctgttgcagctaatgtgcaagagctttgcacttctgcaattaaaaaggcaggggagttgtcagccaatgcaggtagagatttagttccagctgcgtgcaggtttcatttcgaagatattgcaccaaagcaagtgactcttcgtctgattgagctacctagtgctgtagaatatgatgttaagggttacaagttatggtatttcaagaaaggagagatgcctgaggatgatttatttgttgattgcagtagaactgagaggaggatggtgatatctgaccttgagccttgcacggagtacacattccgtgttgtctcttacacagaagctggtatatttggccattcgaacgctatgtgctttacgaagagcgttgagatattgaaaccagtggatggtaaggaaaagagaacaattgatttagtaggtaacgctcagccctcagatagagaggagaaaagtagcatttcctcaagatttcaaattgggcaacttgggaagtatgtgcagttggctgaagctcaggaggaaggcttgcttgaagcgttttacaatgtagatactgagaaaatttgtgagccgccagaggaagaattgccacctcgaaggccacatgggtttgatctaaatgtagtttcagtgccagacttgaatgaggagttcactccacctgattcttctggaggtgaagacaatggagtgccgctaaattcgcttgctgaggctgatggtggtgatcatgatgataactgtgatgatgctgtgtctaacggtagacggaagaacaacaacgactgcttggttatatcagatggaagtggtgatgataccggatttgatttcctcatgaccaggaagaggaaagcaatttcagacagtaatgactcagagaaccacgagtgtgacagttcgtcgattgatgacactcttgagaaatgtgtgaaggtgatcaggtggctggagcgtgaaggccacattaaaacaacattcagggtcaggttcttgacatggttcagcatgagctcaaccgctcaggagcaatctgttgtgagcacatttgtgcagactttagaggatgatccaggtagccttgctggccaacttgtcgacgcatttactgatgttgtctccaccaaaaggccaaacaatggagtaatgacctcacattga

SEQ ID NO: 36 - protein, 602 aa, Arabidopsis thaliana
MDSSSTKSKISHSRKTNKKSNKKHESNGKQQQQQDVDGGGGCLRSSWICKNASCRANVPKEDSFCKRCSCCVCHNFDENKDPSLWLVCEPEKSDDVEFCGLSCHIECAFREVKVGVIALGNLMKLDGCFCCYSCGKVSQILGCWKKQLVAAKEARRRDGLCYRIDLGYRLLNGTSRFSELHEIVRAAKSMLEDEVGPLDGPTARTDRGIVSRLPVAANVQELCTSAIKKAGELSANAGRDLVPAACRFHFEDIAPKQVTLRLIELPSAVEYDVKGYKLWYFKKGEMPEDDLFVDCSRTERRMVISDLEPCTEYTFRVVSYTEAGIFGHSNAMCFTKSVEILKPVDGKEKRTIDLVGNAQPSDREEKSSISSRFQIGQLGKYVQLAEAQEEGLLEAFYNVDTEKICEPPEEELPPRRPHGFDLNVVSVPDLNEEFTPPDSSGGEDNGVPLNSLAEADGGDHDDNCDDAVSNGRRKNNNDCLVISDGSGDDTGFDFLMTRKRKAISDSNDSENHECDSSSIDDTLEKCVKVIRWLEREGHIKTTFRVRFLTWFSMSSTAQEQSVVSTFVQTLEDDPGSLAGQLVDAFTDVVSTKRPNNGVMTSH

SEQ ID NO: 37 - DNA, 1257 bp, Arabidopsis thaliana
  CDS (1)..(1257)
atggaggaaagcaaacagaactatgacctgacgccactaatagcgcctaacctggacagacacttggtgtttcctatattcgagttccttcaagagcgtcagctttaccctgatgagcagatcctgaagtctaaaatccagcttttgaaccagacgaacatggttgattacgccatggatattcacaagagtctctaccacactgaagacgctcctcaagaaatggtggagagaagaacagaggttgtcgctaggctcaaatctttggaggaggctgctgcaccactcgtgtcttttcttttgaaccctaacgctgtgcaggagctaagagctgacaagcagtacaatctccaaatgctcaaggaacgctaccagattggtccagaccagattgaggctttgtaccagtacgccaagtttcagtttgaatgtggcaactattctggtgctgctgattatctttaccagtacaggaccctgtgctctaaccttgagaggagtttgagtgccttgtggggaaagctcgcatctgaaatattgatgcaaaactgggatattgctcttgaagagcttaaccgtctcaaagagattattgactcaaagtttttcatcgccgttaaaccaggtgcagaacaggatttggttgatgcattggggtatctgaatgccatccaaactagtgctccacacttgctgcgctacttggcaactgctttcattgtcaacaaaaggagaagaccacaattgaaagaattcattaaggtcattcagcaagagcactactcctacaaagatccaattatcgagttcctggcatgtgtgtttgtcaattatgactttgatggggctcaaaagaagatgaaagagtgtgaagaggtcattgtgaatgatccattccttggcaagcgagttgaggatggaaacttttcaactgtaccactgagagatgaatttcttgaaaatgcccgcctattcgtctttgaaacctattgcaaaattcatcaaaggattgacatgggggtacttgctgaaaaattgaatctgaactatgaggaggccgagagatggattgtgaacctaatccgcacctcaaagcttgatgccaagattgattctgagtcaggaactgtaatcatggagcctactcagcccaacgtgcatgagcagttgataaaccacaccaaaggcttatcaggacgaacatacaagttagtgaatcagctcttggaacacacacaggcgcaagcaactcgctag

SEQ ID NO: 38 - protein, 418 aa, Arabidopsis thaliana
MEESKQNYDLTPLIAPNLDRHLVFPIFEFLQERQLYPDEQILKSKIQLLNQTNMVDYAMDIHKSLYHTEDAPQEMVERRTEVVARLKSLEEAAAPLVSFLLNPNAVQELRADKQYNLQMLKERYQIGPDQIEALYQYAKFQFECGNYSGAADYLYQYRTLCSNLERSLSALWGKLASEILMQNWDIALEELNRLKEIIDSKFFIAVKPGAEQDLVDALGYLNAIQTSAPHLLRYLATAFIVNKRRRPQLKEFIKVIQQEHYSYKDPIIEFLACVFVNYDFDGAQKKMKECEEVIVNDPFLGKRVEDGNFSTVPLRDEFLENARLFVFETYCKIHQRIDMGVLAEKLNLNYEEAERWIVNLIRTSKLDAKIDSESGTVIMEPTQPNVHEQLINHTKGLSGRTYKLVNQLLEHTQAQATR

SEQ ID NO: 39 - DNA, 4491 bp, Arabidopsis thaliana
  CDS (1)..(4491)
atggatccttcaagacgaccaccgaaggactctccttacgcgaatctattcgatctcgagccgttgatgaagtttagaattccgaaacctgaagatgaagttgattattatgggagtagtagccaggatgaaagtagaagcactcaaggtggggtagtggcaaactacagcaatgggtctaaatcgagaatgaatgcgagctccaagaagagaaagcggtggacagaagctgaggatgcagaggacgatgatgatctctacaatcaacatgttactgaggagcactaccgatcaatgcttggggagcatgtacaaaaattcaaaaataggtccaaggagactcaagggaatcctcctcatctgatgggttttccggtgctaaagagcaatgtgggcagttacagaggtaggaaaccagggaatgattaccatgggaggttctatgacatggacaactctccaaattttgcagctgatgtgaccccacataggcgaggaagctaccatgatcgtgatattacacccaagatagcatatgaaccttcgtatttggacattggtgatggtgtcatctacaaaatccccccaagttatgacaagctggtggcatcattaaacttaccgagcttttcagacattcatgtggaagaattttacttgaaaggaactctggatctgagatcattagcagaactgatggcaagtgataaaaggtctggagtaagaagccgtaatggaatgggtgagcctcgacctcaatatgaatctcttcaagctagaatgaaggccctgtcaccttcaaactccaccccaaattttagcctcaaggtgtcagaagctgcaatgaattctgccattccagaaggatctgctggaagtactgcacggacaattctgtctgagggtggtgttttacaggtccattacgtgaagattctggagaagggggatacatacgagattgttaaacgaagtctaccgaagaagctgaaagcaaagaatgatcctgcagtcattgagaaaacagaaagggataaaattagaaaagcctggatcaatattgtcagaagagatatagcaaaacaccatagaattttcactacttttcatcgtaaactatcaattgatgccaagaggtttgcagatggttgccaaagagaggtgagaatgaaggtgggtagatcatacaaaatcccaagaactgcaccaattcgcactaggaagatatccagagacatgctgctattctggaagcgatatgacaagcagatggcagaagagaggaaaaagcaagaaaaggaagctgcagaggcttttaaacgtgaacaggagcagcgagagtcaaaaaggcagcaacaaaggctcaatttccttattaaacagactgagctttacagtcacttcatgcaaaacaagaccgattcgaatccttccgaagccttaccaataggtgatgaaaatccgattgacgaagtgctcccagaaacttcagcggcagaaccttctgaggtagaggatcctgaagaggctgaactgaaggaaaaggtcttgagagctgcccaagatgcggtgtctaagcagaagcaaataacagatgcatttgacactgaatatatgaagctacgccaaacttctgaaatggaaggtcctttaaatgatatatcagtttctggctcgagcaatatagatttgcataacccatctacaatgcctgttacatcaacagttcagactccagagttatttaaaggaacccttaaagaataccaaatgaaaggccttcagtggctagtcaattgttatgagcagggtttgaatggcatacttgctgatgaaatgggcttgggtaagactattcaagctatggcgttcttggcacatttggctgaggaaaagaacatttggggtccatttcttgttgttgcccctgcctctgttcttaacaattgggctgatgaaatcagtcgtttctgtcctgacttgaaaactcttccatattggggaggattacaagaacgaacaattttaagaaagaatatcaatcccaagcgtatgtaccgaagggatgctggctttcatattttgattactagctatcagctattagtcactgatgaaaagtattttcgccgggtgaagtggcaatatatggtgctagatgaggcccaagcaatcaagagttcctccagtataagatggaaaacccttcttagttttaactgtcggaaccgattgcttctgactggtactccaattcagaacaacatggcagagttatgggccctgctgcatttcatcatgccaatgttgtttgacaaccatgatcaatttaatgaatggttctcaaaaggaattgagaatcatgctgaacacggaggcactttaaatgagcaccagcttaacagactgcatgcgatcttgaaaccgttcatgcttcgacgggtaaaaaaggatgtggtttctgagctaactacaaagacggaagttacagtacactgcaagctcagttctcgacaacaagctttttatcaggctattaagaacaaaatttctctggctgagttgtttgatagcaaccgcggacaatttactgataagaaagtattgaatttaatgaatattgtcattcaactaaggaaggtttgcaaccatccagagttgttcgaaaggaatgaagggagctcgtatctctactttggagtgacttccaattctcttttgccccatccctttggtgagctagaggatgtacattattctggtggtcaaaatccgataatatacaagatacctaagctactacaccaagaggtgctccaaaattctgaaacattttgttcttctgtcgggcgtggcatctcaagagaatcttttctgaagcattttaatatatattcacctgagtatattcttaagtcaatattcccatctgatagtggggtagatcaagtggttagtggaagtggagcatttggcttttcacgcttgatggatctatcaccatcagaagttggatatctggctctgtgttctgttgcagaaaggctattattttctatactgaggtgggagcggcaatttttggatgaattagttaactctcttatggagtccaaggatggtgatcttagtgacaataacatcgagagagttaaaaccaaagctgtcacaagaatgttgctgatgccatcaaaagttgaaacgaattttcagaaaaggagactaagcacagggcctacccgtccttcatttgaagcgctagtgatctctcatcaggataggtttctttcaagtatcaaactcctgcattctgcatatacttatatcccaaaagccagagctccacctgtaagcattcattgctcggacagaaattcggcatacagagttacagaagaattacatcaaccatggcttaagagactattaatcggttttgcacgaacgtcagaagctaatggacccaggaagcctaacagctttccacatcctttaatccaagaaattgattcagaacttccagttgtgcagcctgcgcttcaactgacacacagaatatttggttcttgccctccaatgcaaagttttgacccagcaaagttgctcacggactctgggaagctgcagacacttgatatattattgaagcggcttcgagctggaaatcacagggtgctcctgtttgcacaaatgacaaagatgctgaacattctcgaggattatatgaactatagaaagtacaagtacctcaggcttgatggatcctccaccatcatggatcgccgagatatggttagggattttcagcataggagcgatatttttgtattcttgctgagcaccagagctggaggacttggtatcaacttgacggctgcagacactgtcattttctatgaaagtgattggaatcccaccttggatttacaagctatggacagggctcatcgtcttggacagacaaaagatgagacggtggaagagaaaattttgcacagggcaagtcagaaaaatacagttcaacagcttgttatgactggagggcatgttcagggtgatgattttcttggagctgcggatgtggtatctctgctaatggatgatgcggaggcagcacaactggagcagaaattcagagaactaccattacaggacaggcagaagaaaaagacgaaacgtatcagaatagatgctgaaggagatgcaactttggaagagttagaagatgttgaccgacaggataacggacaggaacctttggaagaaccggaaaagccaaaatccagtaataaaaagaggagagctgcttcaaatccgaaagctagagctcctcagaaagcaaaggaagaagcaaatggtgaagatactcctcagaggacaaaaagggtaaagagacaaacaaagagcataaacgaaagtcttgaacctgtattctctgcctctgtaacagaatcaaataaaggattcgatccaagtagctccgctaactaa

SEQ ID NO: 40 - protein, 1496 aa, Arabidopsis thaliana
MDPSRRPPKDSPYANLFDLEPLMKFRIPKPEDEVDYYGSSSQDESRSTQGGVVANYSNGSKSRMNASSKKRKRWTEAEDAEDDDDLYNQHVTEEHYRSMLGEHVQKFKNRSKETQGNPPHLMGFPVLKSNVGSYRGRKPGNDYHGRFYDMDNSPNFAADVTPHRRGSYHDRDITPKIAYEPSYLDIGDGVIYKIPPSYDKLVASLNLPSFSDIHVEEFYLKGTLDLRSLAELMASDKRSGVRSRNGMGEPRPQYESLQARMKALSPSNSTPNFSLKVSEAAMNSAIPEGSAGSTARTILSEGGVLQVHYVKILEKGDTYEIVKRSLPKKLKAKNDPAVIEKTERDKIRKAWINIVRRDIAKHHRIFTTFHRKLSIDAKRFADGCQREVRMKVGRSYKIPRTAPIRTRKISRDMLLFWKRYDKQMAEERKKQEKEAAEAFKREQEQRESKRQQQRLNFLIKQTELYSHFMQNKTDSNPSEALPIGDENPIDEVLPETSAAEPSEVEDPEEAELKEKVLRAAQDAVSKQKQITDAFDTEYMKLRQTSEMEGPLNDISVSGSSNIDLHNPSTMPVTSTVQTPELFKGTLKEYQMKGLQWLVNCYEQGLNGILADEMGLGKTIQAMAFLAHLAEEKNIWGPFLVVAPASVLNNWADEISRFCPDLKTLPYWGGLQERTILRKNINPKRMYRRDAGFHILITSYQLLVTDEKYFRRVKWQYMVLDEAQAIKSSSSIRWKTLLSFNCRNRLLLTGTPIQNNMAELWALLHFIMPMLFDNHDQFNEWFSKGIENHAEHGGTLNEHQLNRLHAILKPFMLRRVKKDVVSELTTKTEVTVHCKLSSRQQAFYQAIKNKISLAELFDSNRGQFTDKKVLNLMNIVIQLRKVCNHPELFERNEGSSYLYFGVTSNSLLPHPFGELEDVHYSGGQNPIIYKIPKLLHQEVLQNSETFCSSVGRGISRESFLKHFNIYSPEYILKSIFPSDSGVDQVVSGSGAFGFSRLMDLSPSEVGYLALCSVAERLLFSILRWERQFLDELVNSLMESKDGDLSDNNIERVKTKAVTRMLLMPSKVETNFQKRRLSTGPTRPSFEALVISHQDRFLSSIKLLHSAYTYIPKARAPPVSIHCSDRNSAYRVTEELHQPWLKRLLIGFARTSEANGPRKPNSFPHPLIQEIDSELPVVQPALQLTHRIFGSCPPMQSFDPAKLLTDSGKLQTLDILLKRLRAGNHRVLLFAQMTKMLNILEDYMNYRKYKYLRLDGSSTIMDRRDMVRDFQHRSDIFVFLLSTRAGGLGINLTAADTVIFYESDWNPTLDLQAMDRAHRLGQTKDETVEEKILHRASQKNTVQQLVMTGGHVQGDDFLGAADVVSLLMDDAEAAQLEQKFRELPLQDRQKKKTKRIRIDAEGDATLEELEDVDRQDNGQEPLEEPEKPKSSNKKRRAASNPKARAPQKAKEEANGEDTPQRTKRVKRQTKSINESLEPVFSASVTESNKGFDPSSSAN

SEQ ID NO: 41 - DNA, 1815 bp, Arabidopsis thaliana
  CDS (1)..(1815)
atggatcagagaagaggaaatgagcttgatgaatttgagaagcttctaggagagattccaaaagttacttcaggaaacgactataaccatttccctatatgtttgagctcaagcagatcacaatccatcaagaaggttgatcaatatcttcctgatgaccgtgcctttaccacttcattttccgaggctaacttacactttggaatcccaaatcacactccagagtctccccatcctttgttcattaacccttcttaccactcaccaagtaactcaccttgtgtatatgacaagtttgattcaagaaaactcgatccggtaatgttcaggaagctgcaacaagttggataccttccaaacttgtcttcagggatctcacctgctcagcggcagcattacctgccacattcgcagcctctgtctcactatcaatcacctatgacttggagggatatcgaagaagaaaattttcagaggcttaaacttcaagaagaacagtatttgtctattaaccctcatttcctccatcttcagagcatggatactgttccaagacaggaccatttcgattatcgccgagctgaacagtctaacagaaacttgttttggaatggagaagatggtaatgaaagtgtgaggaaaatgtgctatccggagaagattttaatgagatcacagatggatttgaacactgctaaagtcataaagtatggtgctggagatgagtcacaaaatggaagactttggttgcagaatcaactcaatgaagatctcacaatgagtctcaataatctgtcattgcagcctcaaaagtataactctattgcagaggcaagagggaagatatactacttggccaaggatcagcacggttgtcgcttcttgcagagaatattttctgagaaagatgggaatgatatagagatgatctttaatgagatcattgactatatcagtgagctaatgatggatccttttgggaactatttggttcaaaagctgctagaagtatgcaatgaggatcagaggatgcagattgttcattccataactagaaaaccaggactgcttatcaaaatctcttgtgatatgcacgggactagagctgttcaaaagatagttgaaacggctaagagagaggaggagatttcaatcatcatttctgctttgaagcatggcattgtgcatttgataaagaatgtaaacggtaatcacgttgtacaacgatgtttgcagtatctgttaccttactgcggaaagttccttttcgaagctgcgattactcattgtgttgagcttgcaactgatagacatggatgttgtgtacttcaaaaatgtcttggatattcagaaggcgaacaaaagcaacatttagtctctgaaattgcgtccaatgctctactcctctctcaagatccttttggaatagatgcaaactttttttgcaggaactatgtacttcaatatgtctttgagcttcaacttcaatgggcaacctttgaaatcctggagcaattagaaggaaactacaccgagttatcgatgcagaaatgtagcagcaatgtagttgaaaagtgtctgaaactagctgatgacaaacaccgagctcgcatcatcagagaattgattaactatggtcgtcttgatcaagtgatgttggatccttatggaaattatgtcattcaagcagctcttaaacaatccaaggggaatgttcatgctcttttggttgatgccattaaactgaatatctcatctcttcgtaccaatccttacggtaaaaaagtcctctccgcacttagctcgaagaagtaa

SEQ ID NO: 42 - protein, 604 aa, Arabidopsis thaliana
MDQRRGNELDEFEKLLGEIPKVTSGNDYNHFPICLSSSRSQSIKKVDQYLPDDRAFTTSFSEANLHFGIPNHTPESPHPLFINPSYHSPSNSPCVYDKFDSRKLDPVMFRKLQQVGYLPNLSSGISPAQRQHYLPHSQPLSHYQSPMTWRDIEEENFQRLKLQEEQYLSINPHFLHLQSMDTVPRQDHFDYRRAEQSNRNLFWNGEDGNESVRKMCYPEKILMRSQMDLNTAKVIKYGAGDESQNGRLWLQNQLNEDLTMSLNNLSLQPQKYNSIAEARGKIYYLAKDQHGCRFLQRIFSEKDGNDIEMIFNEIIDYISELMMDPFGNYLVQKLLEVCNEDQRMQIVHSITRKPGLLIKISCDMHGTRAVQKIVETAKREEEISIIISALKHGIVHLIKNVNGNHVVQRCLQYLLPYCGKFLFEAAITHCVELATDRHGCCVLQKCLGYSEGEQKQHLVSEIASNALLLSQDPFGIDANFFCRNYVLQYVFELQLQWATFEILEQLEGNYTELSMQKCSSNVVEKCLKLADDKHRARIIRELINYGRLDQVMLDPYGNYVIQAALKQSKGNVHALLVDAIKLNISSLRTNPYGKKVLSALSSKK

SEQ ID NO: 43 - DNA, 2070 bp, Arabidopsis thaliana
  CDS (1)..(2070)
atggcgattattactactactactgttcgtttcactgatggaacctctcccaccttcttctcctcagcttcgacaaaggcttataatctccattttctctactcgaattcaacccaacgacttacgaatccgaaattcggaatcggcgggaagttgaaggtgacggtgaatccgtattcgtatacagaggaagtacggcctgaggaacggaagagtttgacggattttttaacggaagctggagatttcgttaattcagacggcggagatggtggtccgccacggtggttctcaccgttggaatgtggcgcacgtgctcctgaatctcctcttcttctctacttacctgggatcgatggaactggattagggctcattcgccagcataagaggcttggagagatatttgacatatggtgccttcactttccagtaaaagatcgtactcctgctcgagatattgggaagctcattgagaagacagttaggtcagagcactaccgtttcccaaatagacccatttatatagttggagaatctattggagcttctcttgctctggatgttgcagccagtaaccctgacattgatcttgtcttgattctggctaatccagtcacacgttttaccaacttaatgttgcaacctgtattggccctactggaaattttgcctgacggagttcccggcttgataacagagaattttgggttttaccaagcttccccattgacagaaatgttcgagactatgctcaatgaaaatgatgccgcgcagatgggtagagggctattaggagacttctttgcaacttcatctaatctgcctactctgattagaatctttcccaaggacacacttctatggaagcttcaattgcttaagtctgcttcagcgtctgctaattctcagatggacacagtcaacgcccaaacactgatacttctgagtggacgtgatcaatggttaatgaacaaggaagacattgaaagactccgtggtgcattgccaagatgtgaagttcgtgagcttgagaataatggacagttcctcttcttggaggatggagtagatctggtgagtatcatcaagcgtgcgtattattatcgccgtgggaagtcacttgattacatttcggattacattctgcctaccccatttgagtttaaagagtatgaagaatcacaaagattgctaactgctgttacctccccagtctttctttcaactctaaagaatggtgcagtggtaagatcgcttgcaggaataccttcagagggaccggttctgtatgttggcaatcacatgttgcttggtatggagttgcatgcaatagcacttcattttttgaaagaaaggaacattctattgcgaggactggcacatccattgatgtttaccaaaaaaactggctcaaaactccctgacatgcagctgtacgacttatttaggattataggcgcagttcccgtctcgggaatgaatttctacaaactacttcgttcaaaggctcacgtggctttgtaccctgggggtgttcgtgaagctttgcacagaaagggtgaagaatacaagttattttggccagaacattcggagtttgtaaggatagcatctaaatttggagcaaaaatcattccttttggagttgttggagaagatgatctttgtgaaatggttttagattatgatgatcaaatgaagatccctttcttgaagaatcttatagaagagataacacaagactctgttaacttgaggaacgatgaagaaggcgaattgggaaaacaagatttacatctacctggaatagttccaaagatcccgggacggttttacgcatactttgggaaaccaatagacacagaaggtagagagaaagagctaaacaataaagagaaagctcatgaggtttacttgcaggtcaagtctgaggtagaaagatgtatgaactatttgaaaatcaaaagagaaactgatccttacagaaacattttgccgaggtccctctattacctcactcatggtttctcttcccaaatcccaaccttcgatctccgaaatcattaa

SEQ ID NO: 44 - protein, 689 aa, Arabidopsis thaliana
MAIITTTTVRFTDGTSPTFFSSASTKAYNLHFLYSNSTQRLTNPKFGIGGKLKVTVNPYSYTEEVRPEERKSLTDFLTEAGDFVNSDGGDGGPPRWFSPLECGARAPESPLLLYLPGIDGTGLGLIRQHKRLGEIFDIWCLHFPVKDRTPARDIGKLIEKTVRSEHYRFPNRPIYIVGESIGASLALDVAASNPDIDLVLILANPVTRFTNLMLQPVLALLEILPDGVPGLITENFGFYQASPLTEMFETMLNENDAAQMGRGLLGDFFATSSNLPTLIRIFPKDTLLWKLQLLKSASASANSQMDTVNAQTLILLSGRDQWLMNKEDIERLRGALPRCEVRELENNGQFLFLEDGVDLVSIIKRAYYYRRGKSLDYISDYILPTPFEFKEYEESQRLLTAVTSPVFLSTLKNGAVVRSLAGIPSEGPVLYVGNHMLLGMELHAIALHFLKERNILLRGLAHPLMFTKKTGSKLPDMQLYDLFRIIGAVPVSGMNFYKLLRSKAHVALYPGGVREALHRKGEEYKLFWPEHSEFVRIASKFGAKIIPFGVVGEDDLCEMVLDYDDQMKIPFLKNLIEEITQDSVNLRNDEEGELGKQDLHLPGIVPKIPGRFYAYFGKPIDTEGREKELNNKEKAHEVYLQVKSEVERCMNYLKIKRETDPYRNILPRSLYYLTHGFSSQIPTFDLRNH

SEQ ID NO: 45 - DNA, 1038 bp, Arabidopsis thaliana
  CDS (1)..(1038)
atggaagaactgaaagtggaaatggaggaagaaacggtgacgtttactggttctgtagcggcttcttcatctgtaggatcctcttcctctcctagaccaatggaagggcttaacgaaacagggccaccaccgtttctgactaagacttacgaaatggtggaagatccggcgacggacacggtggtttcttggagtaatggtcgtaacagctttgtggtgtgggattctcataagttctcaacaactctccttccacgttacttcaagcatagcaatttctcaagttttattcgtcagctcaatacttatggattcagaaagattgatccagatagatgggaatttgcaaatgaagggtttttagcaggacaaaagcatctcttgaagaacatcaaaagaaggaggaacatgggtttgcagaatgtgaatcagcaaggatctgggatgtcatgtgttgaggttgggcaatacggtttcgacggggaggttgagaggttgaagagggatcatggtgtgcttgtagctgaggtagttaggttgaggcaacagcaacacagctccaagagtcaagttgcagctatggagcaacggttgcttgttactgagaagagacagcagcagatgatgacgttccttgccaaggcgttgaacaatccgaactttgttcagcagtttgcggttatgagtaaagagaagaagagtttgtttggtttggatgtggggaggaaacggaggcttacttctactccaagcttggggactatggaggagaatttgttacatgatcaagagtttgatagaatgaaggatgatatggaaatgttgttcgctgcagcaatcgatgatgaggcgaataattcgatgcctactaaggaggaacaatgtttggaggctatgaatgtgatgatgagagatggtaatttggaagcagcgttggatgtgaaagtggaagatttggttggttcgcctttggattgggacagccaagatctacatgacatggttgatcaaatgggttttcttggttcggaaccttaa

SEQ ID NO: 46 - protein, 345 aa, Arabidopsis thaliana
MEELKVEMEEETVTFTGSVAASSSVGSSSSPRPMEGLNETGPPPFLTKTYEMVEDPATDTVVSWSNGRNSFVVWDSHKFSTTLLPRYFKHSNFSSFIRQLNTYGFRKIDPDRWEFANEGFLAGQKHLLKNIKRRRNMGLQNVNQQGSGMSCVEVGQYGFDGEVERLKRDHGVLVAEVVRLRQQQHSSKSQVAAMEQRLLVTEKRQQQMMTFLAKALNNPNFVQQFAVMSKEKKSLFGLDVGRKRRLTSTPSLGTMEENLLHDQEFDRMKDDMEMLFAAAIDDEANNSMPTKEEQCLEAMNVMMRDGNLEAALDVKVEDLVGSPLDWDSQDLHDMVDQMGFLGSEP

SEQ ID NO: 47 - DNA, 1179 bp, Arabidopsis thaliana
  CDS (1)..(1179)
atgatcgttctttttcttcaaatcattacatgttctctcttcacgaccactgcctcatcacctcacggcttcaccattgacttgatccagcgtcgttcgaattcatcttcttctcgactgtccaaaaatcagttgcaaggagcatcaccttacgccgatactttatttgactacaacatctatctaatgaaactacaagtcggtactcctcctttcgagatcgaagcggagatagacacaggaagtgacctcatatggacacaatgtatgccttgtactaactgctacagccaatacgctcctatattcgacccttcgaattcttcaaccttcaaagaaaaaagatgcaacgggaactcttgtcattacaagattatctacgcggacacaacctattccaagggaaccttggcaaccgagacggtcacgatccattccacttcaggggaaccctttgtgatgcctgaaaccactattggttgtggccacaacagctcatggtttaaacctactttttcgggcatggttggtctaagctggggaccttcatcgctcatcactcagatgggcggtgagtacccaggtttgatgtcttactgttttgctagtcaaggaactagtaagatcaattttggaacaaatgctattgttgcaggagatggggttgtatcaaccactatgtttctcacgacggcgaaaccaggtttatattacctaaatctagacgcggtcagcgttggggacacccatgttgagacaatggggacaacgtttcatgcgttagaagggaacataattatagactctggaaccactctaacctactttcctgtgagctactgcaacctagtaagagaggcagtggatcattatgtgacagcggttcgaacagccgaccctaccggcaatgacatgctttgctactacacggacaccatagatatctttcccgtgatcacaatgcatttttctggcggtgcggatcttgtcttggataagtataacatgtatatcgaaacgattacgagaggaaccttttgtctggctattatatgtaataatccaccacaagatgctatctttgggaacagagcacagaacaattttttggtgggttatgattcttcttcacttttggtttctttcagtcccaccaattgttctgcattgtggaattga

SEQ ID NO: 48 - protein, 392 aa, Arabidopsis thaliana
MIVLFLQIITCSLFTTTASSPHGFTIDLIQRRSNSSSSRLSKNQLQGASPYADTLFDYNIYLMKLQVGTPPFEIEAEIDTGSDLIWTQCMPCTNCYSQYAPIFDPSNSSTFKEKRCNGNSCHYKIIYADTTYSKGTLATETVTIHSTSGEPFVMPETTIGCGHNSSWFKPTFSGMVGLSWGPSSLITQMGGEYPGLMSYCFASQGTSKINFGTNAIVAGDGVVSTTMFLTTAKPGLYYLNLDAVSVGDTHVETMGTTFHALEGNIIIDSGTTLTYFPVSYCNLVREAVDHYVTAVRTADPTGNDMLCYYTDTIDIFPVITMHFSGGADLVLDKYNMYIETITRGTFCLAIICNNPPQDAIFGNRAQNNFLVGYDSSSLLVSFSPTNCSALWN

SEQ ID NO: 49 - DNA, 4539 bp, Arabidopsis thaliana
  CDS (1)..(4539)
atggagacaaaagttgggaagcaaaagaagagaagtgttgactcaaatgatgatgtctctaaggaaaggagaccaaagcgagcagcagcttgcagaaacttcaaggagaaacctcttcgtatctctgacaaatctgaaaccgttgaagctaagaaagagcagaacgtggtggaagagatcgtggcgatacagttaacttcttctttggagagcaatgatgatcctcgtccaaaccggaggctgactgattttgttttacataattcagatggagttccacagcctgtggagatgttggaacttggtgacatttttcttgaaggtgttgtcttacctttaggtgatgacaaaaacgaagaaaagggtgtgaggtttcaatcttttggtcgtgtcgagaactggaatatatctggttatgaagatggttccccggggatatggatatcaacagcgttagcggattacgattgccgtaaaccagcttctaaatacaagaaaatatatgattatttctttgagaaagcttgtgcttgtgtggaggtgtttaagagcttgtccaagaatccggatacaagtcttgatgagcttcttgcggcggttgcgaggtcgatgagcggaagcaagatattttctagcggtggagccatccaagagtttgttatatcccaaggagaattcatatataaccaactcgctggtctggatgagacagccaagaatcatgaaacatgctttgttgaaaattctgttcttgtttctctaagagatcatgaaagtagtaaaatccacaaggctttgtctaatgtggctctgaggattgatgagagccagctcgtgaaatctgatcatttagtggatggtgctgaggccgaggatgtaagatatgctaagttaatccaagaagaagagtatcggatatctatggagcggtcgagaaataagagaagttcaacaacttctgcttcgaataagttttacattaagatcaatgaacacgagattgccaatgattatccactcccgtcttactacaagaacaccaaagaagaaacagatgagcttttactctttgaacctggctatgaggtagatacaagggacctaccttgtagaacacttcacaattgggctctttacaactctgattcacggatgatatcattagaggttcttcccatgaggccgtgtgctgaaatcgatgtcaccgtatttgggtcaggtgtggtggctgaagatgatggaagtgggttttgtctcgatgattcagagagctctacctctacgcagtcaaatgttcatgatgggatgaacatattccttagtcaaataaaggaatggatgattgagtttggagcagaaatgatctttgtcacattacgaactgacatggcctggtatcgacttgggaaaccgtcaaagcaatatgctccatggtttgaaactgttatgaaaacagtaagggttgcgataagcattttcaatatgctcatgagagaaagtagggttgctaagctttcatatgcaaatgtcataaaaagactttgtgggttagaggagaacgataaagcttacatttcttctaagctcttggatgttgagagatatgttgtcgtccatggacaaattatcttgcagcttttcgaagagtatcctgacaaggatatcaaaaggtgtccatttgttactggtcttgcaagtaaaatgcaggatatacaccacacaaaatggatcatcaagaggaagaagaaaattctgcaaaagggaaagaatctgaatccgagggcgggcttggcacatgtggtaaccagaatgaaacctatgcaagcaacaacaactcgcctcgttaatagaatttggggagagttttactccatttactctcctgaggttccatcggaggcgattcatgaagtggaagaagaggagattgaagaggatgaagaggaggacgagaatgaggaagatgatatagaggaggaagctgttgaggttcaaaagtctcatactcctaagaaaagtagaggtaattctgaagatatggagataaaatggaatggtgagattcttggagaaacttctgatggtgagcctctctatggaagagcccttgttggaggggaaacagtggcggtaggtagtgctgtcatattagaagttgatgatccagatgaaactccggcgatctattttgtggagttcatgttcgagagttcagatcagtgcaagatgctacatgggaaactcttacaaagaggatctgagactgttataggaacggctgctaacgagagggaactgttcttgactaatgaatgtcttactgtccatcttaaggacataaaaggaacagtaagtctcgatattcgatcaaggccgtgggggcatcagtataggaaagagaacctcgttgtggataagcttgaccgggcaagagcagaagaaagaaaagctaatggtttgccaacagaatactactgcaaaagcttgtactcacctgagagaggtggattctttagtcttccaaggaatgatattggtcttggttctggattctgtagttcgtgtaagataaaagaggaagaagaggaaaggtccaaaactaaactcaacatctcaaagacaggggttttctccaatgggatagagtattataatggagattttgtctatgtactccccaactacataactaaagatggattgaagaagggtactagtagaagaacaactcttaagtgtggtcggaacgttgggttaaaagcttttgttgtttgccaattgctggatgttattgttctagaagaatctagaaaagctagtaatgcttcatttcaggttaaactgacaaggttttataggcccgaggacatttctgaagaaaaggcttatgcttcagacatccaagagttgtattatagccatgacacatatattcttcctcctgaggctctacaaggaaaatgtgaagtaaggaagaaaaatgatatgcccctatgtcgtgagtatccaatattagatcatatcttcttctgtgaagttttctatgattcctctactggttatctcaagcagtttccagcgaatatgaagctgaagttctctactattaaagatgaaacacttctaagagaaaagaaggggaagggagtagagactggaactagttctggaattcttatgaagcctgatgaggtacctaaagagatgcgtctagctacactagatatttttgctggatgtggtggtctatctcatggactagaaaaggctggtgtatctaatacaaagtgggcgatcgagtatgaagagccagctggtcatgcgtttaaacaaaaccatcccgaagcaacggtttttgttgacaactgcaatgtcattcttagggctataatggagaaatgtggagatgtcgatgattgtgtctctactgtggaggcagctgaacttgtagctaaacttgatgagaaccaaaagagtaccctgccacttcctggtcaagcggatttcatcagcggagggcctccatgccaagggttttctggtatgaacaggttcagtgacggttcgtggagtaaagtacagtgtgaaatgatattagcattcttgtcctttgctgattatttccgaccaaagtattttcttctcgagaacgtaaagaaatttgtgacatacaataaagggagaacatttcaacttactatggcttctcttcttgaaataggttaccaagtaagatttggaatcttggaggcaggtacatatggagtttctcagcctcgtaaaagagttataatttgggcagcttcaccagaagaagttcttccagaatggcctgagccgatgcatgtctttgataatccgggtagtaaaatctccttacctcgaggtttacattatgatactgttcgtaatactaaatttggcgcaccgttccgctcaatcacggtgagagacacaatcggcgatcttccactagtagaaaacggagagtccaagataaacaaagagtatagaactactccagtctcgtggttccaaaagaagataagaggaaacatgagtgttctcactgatcatatctgcaaagggctgaatgaactaaacctcattcgatgtaagaaaatcccaaagaggcctggtgctgattggcgtgacctgccggacgaaaacgtgacattatcaaatggactcgtggaaaaactgcgtcctttagctctatcaaagacagctaaaaaccacaacgaatggaagggactctatggtagattggactggcaaggaaacttacccatttccatcaccgatccgcagcccatgggtaaggtgggaatgtgcttccatccagaacaggacagaattatcactgtccgtgaatgcgcccgatctcaggggtttccggatagctatgagttttcagggacgacaaaacacaaacataggcagattggaaatgcagtccctccaccattggcattcgctctcggtcggaagctcaaagaagccctatatctcaagagttctcttcaacaccaatcataa

SEQ ID NO: 50 - protein, 1512 aa, Arabidopsis thaliana
METKVGKQKKRSVDSNDDVSKERRPKRAAACRNFKEKPLRISDKSETVEAKKEQNVVEEIVAIQLTSSLESNDDPRPNRRLTDFVLHNSDGVPQPVEMLELGDIFLEGVVLPLGDDKNEEKGVRFQSFGRVENWNISGYEDGSPGIWISTALADYDCRKPASKYKKIYDYFFEKACACVEVFKSLSKNPDTSLDELLAAVARSMSGSKIFSSGGAIQEFVISQGEFIYNQLAGLDETAKNHETCFVENSVLVSLRDHESSKIHKALSNVALRIDESQLVKSDHLVDGAEAEDVRYAKLIQEEEYRISMERSRNKRSSTTSASNKFYIKINEHEIANDYPLPSYYKNTKEETDELLLFEPGYEVDTRDLPCRTLHNWALYNSDSRMISLEVLPMRPCAEIDVTVFGSGVVAEDDGSGFCLDDSESSTSTQSNVHDGMNIFLSQIKEWMIEFGAEMIFVTLRTDMAWYRLGKPSKQYAPWFETVMKTVRVAISIFNMLMRESRVAKLSYANVIKRLCGLEENDKAYISSKLLDVERYVVVHGQIILQLFEEYPDKDIKRCPFVTGLASKMQDIHHTKWIIKRKKKILQKGKNLNPRAGLAHVVTRMKPMQATTTRLVNRIWGEFYSIYSPEVPSEAIHEVEEEEIEEDEEEDENEEDDIEEEAVEVQKSHTPKKSRGNSEDMEIKWNGEILGETSDGEPLYGRALVGGETVAVGSAVILEVDDPDETPAIYFVEFMFESSDQCKMLHGKLLQRGSETVIGTAANERELFLTNECLTVHLKDIKGTVSLDIRSRPWGHQYRKENLVVDKLDRARAEERKANGLPTEYYCKSLYSPERGGFFSLPRNDIGLGSGFCSSCKIKEEEEERSKTKLNISKTGVFSNGIEYYNGDFVYVLPNYITKDGLKKGTSRRTTLKCGRNVGLKAFVVCQLLDVIVLEESRKASNASFQVKLTRFYRPEDISEEKAYASDIQELYYSHDTYILPPEALQGKCEVRKKNDMPLCREYPILDHIFFCEVFYDSSTGYLKQFPANMKLKFSTIKDETLLREKKGKGVETGTSSGILMKPDEVPKEMRLATLDIFAGCGGLSHGLEKAGVSNTKWAIEYEEPAGHAFKQNHPEATVFVDNCNVILRAIMEKCGDVDDCVSTVEAAELVAKLDENQKSTLPLPGQADFISGGPPCQGFSGMNRFSDGSWSKVQCEMILAFLSFADYFRPKYFLLENVKKFVTYNKGRTFQLTMASLLEIGYQVRFGILEAGTYGVSQPRKRVIIWAASPEEVLPEWPEPMHVFDNPGSKISLPRGLHYDTVRNTKFGAPFRSITVRDTIGDLPLVENGESKINKEYRTTPVSWFQKKIRGNMSVLTDHICKGLNELNLIRCKKIPKRPGADWRDLPDENVTLSNGLVEKLRPLALSKTAKNHNEWKGLYGRLDWQGNLPISITDPQPMGKVGMCFHPEQDRIITVRECARSQGFPDSYEFSGTTKHKHRQIGNAVPPPLAFALGRKLKEALYLKSSLQHQS

SEQ ID NO: 51 - DNA, 741 bp, Arabidopsis thaliana
  CDS (1)..(741)
atggagtgggagaaatggtacttagatgcggttcttgtgccaagtgctttacttatgatgtttggttaccacatctatttgtggtataaggttcgaaccgatcctttctgcaccattgttggtacaaattcccgcgcccgtcgatcttgggtagcagccatcatgaaggacaacgagaagaagaacatcttagcggtacaaacactacgaaacacgataatgggagggacgttaatggcaaccacttgcatcctcctctgcgcaggtctcgctgccgttttaagcagtacttatagcatcaagaaacctttaaacgacgccgtatatggagctcatggtgacttcactgttgcactcaaatacgtaaccatcctcacaatcttcctcttcgccttcttctctcattctctctccattcgcttcatcaaccaagtcaacatccttattaacgctcctcaagaacctttttctgatgatttcggcgaaataggaagctttgtgactcccgagtatgtctctgaactactcgagaaagctttcttgctcaatacggtaggtaataggctgttctacatgggcttgcctttgatgctatggatctttgggcctgtgcttgtgttcttgagctctgctttgataatccctgttctttataacctcgacttcgtgtttttgttgagcaataaggagaagggtaaagtcgattgcaatggaggttgtgatgacaacttctcgccttaa

SEQ ID NO: 52 - protein, 246 aa, Arabidopsis thaliana
MEWEKWYLDAVLVPSALLMMFGYHIYLWYKVRTDPFCTIVGTNSRARRSWVAAIMKDNEKKNILAVQTLRNTIMGGTLMATTCILLCAGLAAVLSSTYSIKKPLNDAVYGAHGDFTVALKYVTILTIFLFAFFSHSLSIRFINQVNILINAPQEPFSDDFGEIGSFVTPEYVSELLEKAFLLNTVGNRLFYMGLPLMLWIFGPVLVFLSSALIIPVLYNLDFVFLLSNKEKGKVDCNGGCDDNFSP